(19) United States
(12) Patent Application Publication
(10) Pub. No.: US 2017/0107490 A1
(43) Pub. Date: Apr. 20, 2017

(54) EXPANSION OF LYMPHOCYTES WITH A CYTOKINE COMPOSITION FOR ACTIVE CELLULAR IMMUNOTHERAPY

(71) Applicant: POLYBIOCEPT AB, Stockholm (SE)

(72) Inventor: Markus MAEURER, Äkersberga (SE)

(73) Assignee: POLYBIOCEPT AB, Stockholm (SE)

(21) Appl. No.: 15/317,788

(22) PCT Filed: Jun. 11, 2015
(86) PCT No.: PCT/EP2015/063108

§ 371 Date: Dec. 9, 2016

(30) Foreign Application Priority Data
Jun. 11, 2014 (DE) 10 2014 211 167.6

(51) Int. Cl. C12N 5/0783; A61K 35/17

(52) U.S. Cl. CPC C12N 5/0636; A61K 35/17; A61K 2035/124

(57) ABSTRACT

The present invention relates to a composition for expanding lymphocytes comprising at least two types of cytokines selected from interleukin 2 (IL-2), interleukin 15 (IL-15) and interleukin 21 (IL-21). It further relates to a Method of preparing a population of clinically relevant lymphocytes, comprising the steps of: obtaining a body sample from a mammal in particular a tissue sample or body liquid sample, comprising at least one lymphocyte and optionally separating the cells in the body sample, culturing the body sample in-vitro to expand and/or stimulate lymphocytes in the sample wherein the culturing comprises using IL-2, IL-15 and/or IL-21, and optionally determining the presence of clinically relevant lymphocyte in the cultured sample. The present invention also relates to an immunotherapy and the population of clinically relevant lymphocytes.

### FIELD OF THE INVENTION

The present invention relates to active cellular immunotherapy including a method of preparing a population of clinically relevant lymphocytes using a composition of predefined cytokines. The invention further relates to the composition of cytokines and the generated clinically relevant lymphocytes.

### BACKGROUND OF THE INVENTION

Cancer remains to be one of the most common causes of death in the developed countries. For example in the United States and Germany it is the second most common cause of death with a mortality of 560,000 (2009) and 218,000 (2010), respectively. Survival rates remain poor for many cancers despite improvement in the ability to detect and treat this constellation of diseases.

Among the cancer diseases, pancreatic cancer is the fourth leading cause of cancer death in the United States and Sweden without signs of improvement. By the time of diagnosis of pancreatic cancer the majority of patients is incurable with a locally advanced cancer or metastatic disease only allowing for a palliative treatment. The medium survival is about six months. Only about 15 to 20% of the patients have a resectable tumor and therefore a potentially curable disease. Like most cancer, pancreatic cancer is a systemic disease that requires early and systemic intervention. In comparison with many other types of cancer, pancreatic cancer is highly chemotherapy resistant and radiation resistant. In order to achieve a significant breakthrough in improving the dismal prognosis of pancreatic cancer finding new alternatives and more efficient treatment concepts is imperative. The biology of pancreatic cancer is associated with both local and systemic immunosuppression enabling tumor progression and metastasizing.

The situation is similar for glioblastoma, which is the most frequent and progressive glioma with an incidence of 2-3/100.000 in the United States. Glioblastoma make up 12 to 15% of all intracranial and 50 to 60% of histiocytic tumors. New treatment regimens have increased a medium overall survival (14.6 months with radio plus temozolomide as compared to 12.1 months with radio therapy alone). So far intents to develop robust and clinically effective immunotherapeutic protocols have been frustrating for patients with glioblastoma or patients with pancreatic cancer. One approach for treating such cancers is to overcome the tumor induced suppression and/or to induce anti-tumor directed cellular and humoral immune responses.

One of the most promising advances is a new therapeutic class called active cellular immunotherapy (ACI). Cancer immunotherapies can be either passive or active. Passive therapy is based on the adoptive transfer of immunomodulators including cytokines, tumor specific antibodies or immune cells. These substances or cells are then administered to the patient to initiate an anti-tumor action. In general these therapies do not generate immunologic memory and therefore require chronic infusion based treatment. Active immunotherapies, on the other hand, stimulate the patient's immune system with the intent of promoting an antigen specific anti-tumor effect using the body's own immune cells. In addition active immunotherapies seek to create durable anti-tumor response that can protect against minimal residual disease and tumor recurrences.

Clinically relevant and a long-term remission using T-cells directed against tumors (tumor reactive T-cells) have been achieved in patients with melanoma (2, 3). A landmark article showed recently that the best and durable response in cancer treatment is achieved if the patient's own T-cells are directed against the patient's own tumor cells, i.e. the patient's own “private” mutations (4). Such promising results were also obtained for patients with epithelial tumors, i.e. by adaptive transfer of T-cells targeting mutant epitopes in epithelial cancer (5). These approaches usually rely on the harvesting of tumor infiltrating lymphocytes (TIL) from tumor lesions or T-cells from peripheral blood.

A recent report of the CTEP Subcommittee on Adaptive Cell Therapy summarized protocols on the expansion of tumor reactive T-cells from peripheral blood and TILs.

This study compiled the roadmap for using TIL therapy or T-cell based therapy with a particular focus on product consistency and effective yield of the T-cell products. Both consistency and yield for anti-melanoma directed T-cells appear to be achievable with current methodologies that would enable T-cell based strategies to enter the mainstream of cancer treatment, along with the biologicals, i.e. anti-CD40L or PD-1 directed therapies.

Minimally cultured TIL appears to provide the most effective phenotype and profile for clinical use (11). The most successful approach up to date has been the use of autologous ex vivo activated T-cells that were grown in 24 well plates, tested for immune effector functions and further expansion using IL-2, allogeneic feeder cells and OKT3 (9, 12, 13).

CD4+ or CD8+ tumor antigen (TAA) directed T-cells have been generated under GMP conditions from peripheral blood and formulated to subsequent treatment of patients. This has been achieved with autologous CD4+ T-cells (14-16) or CD8+ T-cells(17), some directed against the NY-ESO-1 antigen(18), which is also possible in PBMCs from healthy patients without cancer, since sufficient T-cell precursors are present in the peripheral circulation. Various methods for expansion of T-cells generating CD8+ T-cell clones for target directed therapy (19) have been described. This if great interest since clonal repopulation of the patient's immune system with anti-tumor lymphocytes has been shown to induce cancer regression but also autoimmunity (20).

The growth medium formulation may also be relevant for successful active immunotherapy. Studies showed that starvation impacts on T-cell mediated immune responses and may induce starvation-induced immunosuppression, yet also the expansion of certain T-cell subsets This mechanism appears to be mediated by Leptin (21) which modulates also B-cell development and subsequent B-cell responses (22). This lead to the discovery of the nutrient sensor pathways (i.e. the GCN2 in dendritic cells) that enhances antigen presentation (23). More recent studies showed that cytokine-driven T-cell expansion (such as those in ex vivo expansion of TIL or ACT) are dependent on exogenous amino acids and that cytokines, i.e. IL-7, upregulated genes associated with amino acid transporter expression. Tailoring growth media requirements will therefore be shaped by the respective cytokine cocktail used for T-cell expansion (24) as well as for amino acids in the medium; both factors will impact on T-cell maturation and differentiation which is clinically meaningful.

Clinical (antitumor) efficacy appears to be mediated by CD8+ and central memory cells, defined by CD45RA−CCR7+, defined ex vivo from patients responding to T-cell based therapy. The phenotype of such T-cells is determined by the ex vivo expanded T-cell population, as well as by host factors after adoptive transfer. A diverse population of T-cells targeting cancer cells may be advantageous for effective immune responses, including long-term memory T-cells, yet also T-cells that can immediately react to (cancer) target cells and produce anti-tumor directed immune responses, including terminally differentiated T-cells that express cytolytic molecules, such as granzyme and perforin (25, 26). Long term-memory immune memory is in part determined by the increased proliferation potential and half-life that can be measured by the telomere length (27, 28).

Relatively little is known about what stages of (melanoma)-specific TIL or T-cell clones are best for in vivo transfer, due to gene expression differences in vitro and in vivo, as well as the differential cytokine milieu in individual patients. Not only an individual phenotype, and also rather different phenotypes associated with the fast delivery of immune effector functions (terminally differentiated CD45RA+ CCR7−) T-cells along with the provision of long-term immunologically memory of central memory T-cells that would replenish the differentiated T-cell pool could represent a good choice for expansion of T-cells.

Similarly relevant appear to be the expression of activation/exhaustion markers, i.e. LAG-3, PD-1 and or 4-1 BB on T-cells that may indicate a higher change for T-cell exhaustion and loss of function, and also an enrichment of tumor-antigen specific T-cells (which tend to be PD1+, and/or LAG-3, 4-1 BB+ (29)).

NY-ESO-1 was known to be an eligible target for tumor-antigen specific T-cells. NY-ESO-1 is a cancer testing antigen (36, 37) and expressed in a high number of tumors. For example, at Karolinska 50 glioblastoma lesions were screened for NY-ESO-1 protein expression and it was found that 35% of the grade 3 and 4 GB are positive for NY-ESO-1. Screening of pancreatic cancer lesions showed a lower number of NY-ESO-1 protein+cancer lesions in the range of 20%, particularly in metastatic lesions. Targeting NY-ESO-1 for the expansion of tumor-reactive T-cells from peripheral blood appears to be a ‘safe choice’ of target, since NY-ESO-1 appears to be expressed only in malignant cells and testis without overt ‘off-target’ reactivity in anti-NY-ESO-1 directed T-cells (36). This is of great interest since clonal repopulation of the patients immune system with antitumor lymphocytes has been shown to induce cancer regression, and autoimmunity(20), a potential risk. NYESO-1 has been tested in a number of studies as a potential target in GB, as well as in GB-stem cells (38), along with the use of DNA-methylating agents to increase NY-ESO-1 reactivity (39, 40).

In view of this state of the art it is an objective of the present invention to provide improved methods in immunotherapy.

### SUMMARY OF THE INVENTION

The present invention is inter alia based on the finding that a composition comprising cytokines interleukin-2 (IL-2), interleukin-15 (IL-15) and/or interleukin-21 (IL-21) leads to a superior stimulation and expansion of lymphocytes in particular clinically relevant lymphocytes. The expansion and stimulation procedure with the cytokine mixture is highly sensitive and allows the preparation of the population of clinically relevant lymphocytes even if the starting concentration in the sample is very low.

Thus, according to a first aspect the invention provides a composition for expanding lymphocytes comprising at least two types of cytokines selected from interleukin 2 (IL-2), interleukin 15 (IL-15) and interleukin 21 (IL-21).

With this cytokine composition the inventors were able to determine a novel method for the preparation of population of antigen edited lymphocytes. Therefore according to a second aspect the present invention provides a method of preparing a population of clinically relevant lymphocytes, comprising the steps of:
obtaining a body sample from a mammal, in particular a tissue sample or body liquid sample, comprising at least one lymphocyte and optionally separating the cells in the body sample,
culturing the body sample in-vitro to expand and/or stimulate lymphocytes in the sample, wherein the culturing comprises using IL-2, IL-15 and/or IL-21,
and optionally determining the presence of clinically relevant lymphocyte in the cultured sample.

The method of the second aspect of the invention leads to the formation of population of lymphocytes, which includes a population of clinically relevant lymphocytes.

According a third aspect, the invention provides a clinically relevant lymphocyte obtained by the method according to the second aspect wherein the clinically relevant lymphocyte is selected from a B-cell, an NK cell and T-cell.

According to a fourth aspect the present invention provides a population of lymphocytes obtained by the second aspect of the invention comprising a population of clinically relevant lymphocytes.

The population of clinically relevant lymphocytes obtained with the method according to the second aspect of the invention is in particular advantageous for cellular immunotherapy.

According to a fifth aspect the invention provides an immunotherapy for treating or preventing a tumor disease, an infectious disease or an autoimmune disease in a mammal comprising the steps of generating a clinically relevant lymphocyte population according to the second aspect of the invention wherein the body sample is obtained from said mammal and administering the clinically relevant lymphocyte population to said mammal.

According to a sixth aspect the invention provides a composition according to the first aspect of the invention for use in the medical treatment, in particular for treating and preventing an infectious disease, an autoimmune disease or a tumor disease.

Therefore, according to a seventh aspect the invention provides a kit for use in medical treatment, in particular for treating or preventing an infectious disease, an autoimmune disease or a tumor disease, wherein the kit comprises IL-2, IL-15 and IL-21, and optionally at least one of a component that stimulates the TCR, in particular OKT3, costimulatory molecules, feeder cells and a peptide comprising the amino acid sequence of at least clinically relevant antigen

### FIGURES

FIG. 1 shows three graphs representing the results of flow cytometry analysis of samples from an expansion of PBMCs with the cytokine cocktail IL-2, IL-15 and IL-21 in combination zoledronic acid. The samples were taken at different time points as indicated above the graphs. The measured signals are a CD3 signal in direction of the y-axis and a TCR gamma delta signal in the direction of the x-axis. Gamma delta T-cells are found in the rectangle area. The originally colored images show intensities of overlapping signals in grey scale. The percentage of cells in the rectangle area is shown above.

FIG. 2 shows the result of a flow cytometry analysis of lymphocytes from PBMCs expanded with the cytokine cocktail in the presence of PRDM2 peptides. The larger left panel shows the result of the sample at the beginning of the lymphocyte expansion, the right panel the result of the sample after 18 days of stimulation. Cell signals are separated based on the CD4/CD8 markers. The small panels on the right show the gating for lymphocytes and CD3+ cells.

FIG. 3 shows the flow cytometry analysis of the same samples as FIG. 2 Separation of the cell signals via IFN-γ marker and size (side-scattered light SSC).

FIG. 4 shows the results of a flow cytometry on samples of an expansion of PBMCs with a cytokine cocktail and stimulation with INO80E and UCHL3. The cell signals are gated for lymphocytes (4a), CD3+ (4b) and then separated based on the CD8 and CD4 signal (4c).

FIG. 5 shows the IFN-γ production on the double negative and CD8+ population after stimulation with INO80E or UCHL3.

FIG. 6 shows the analysis of PBMC cells expanded with the cytokine cocktail and INO80E and UCHL3 peptides. Cells stimulated with INO80E were analyzed for the production of cytokines CD107a (6d), CD127 (6e) and CD117 (6f).

FIGS. 7a to 7f show the results of the expansion of PBMCs with the cytokine cocktail and stimulation with CMVpp65.

FIG. 8 shows results of IFN-γ analysis after the stimulation of expanded cells with NY-ESO-1: FIGS. 8a and 8c unstimulated on day 0 and day 18, respectively. FIGS. 8b and 8d stimulated with NY-ESO-1 on day 0 and day 18, respectively.

FIG. 9 shows the cytokine production of cells expanded from PBMCs obtained from a patient with glioblastoma upon stimulation with survivin again on day 0 and day 18. The measured cytokines are IL-2, IFN-γ and TNF-α. FIG. 9a shows the results for the subset of CD4+ T-cells, FIG. 9b for the subset of double negative T-cells and FIG. 9c for the subset of CD8+ T-cells.

FIG. 10 shows the analysis of the phenotypes CD45RA and CCR7 of lymphocytes using flow cytometry. Again lymphocytes are measured on day 0 and after 18 days of expansion with the cytokine cocktail.

FIG. 11 shows the analysis of the effect of expansion on the phenotypes of CD4+ cells (TH1/TH2) and CD8+ T-cells.

FIG. 12 shows the analysis of cytokine CD107a expression by cells expanded from peripheral blood of a patient with HPV. FIG. 12a shows the CD107a expression upon HPV L1 peptide stimulation, FIG. 12b the positive control and FIG. 12c represents the result without stimulation (medium). The gating process for CD8+ T-cells is shown in FIGS. 12d to 12f.

FIG. 13 shows two graphs representing the IFN-γ production of lymphocytes expanded with either no cytokine, IL-2, IL-15, IL-21 or IL-7 and IL-2 and stimulation with NY-ESO-1 or survivin.

FIG. 14 shows three graphs representing the IFN-γ production of lymphocytes expanded with either no cytokine, IL-2, IL-15, IL-21 or IL-7 and IL-2 and stimulation with EBNA-1, EBNA-3a, or CMVpp65.

FIG. 15 shows a flow cytometry analysis determining Treg (regulatory T-cells) were identified prior to and after expansion of T-cells with the cytokine cocktail. From left to right: T-cells were gated on CD4+ T-cells and then on CD25high designating the high expression of the IL-2 receptor on activated T-cells. Then the cells were gated on II-2R (high CD125) cells and tested for expression of the IL-7Receptor (CD127) and Foxp3 (intracellularly).

FIG. 16 shows a flow cytometry analysis determining the percentage of PD-1+ T-cells in the CD8+ subset.

FIG. 17 shows the specific lysis of autologous B-cells pulsed with and pulsed with peptides 1-12 by expanded lymphocytes.

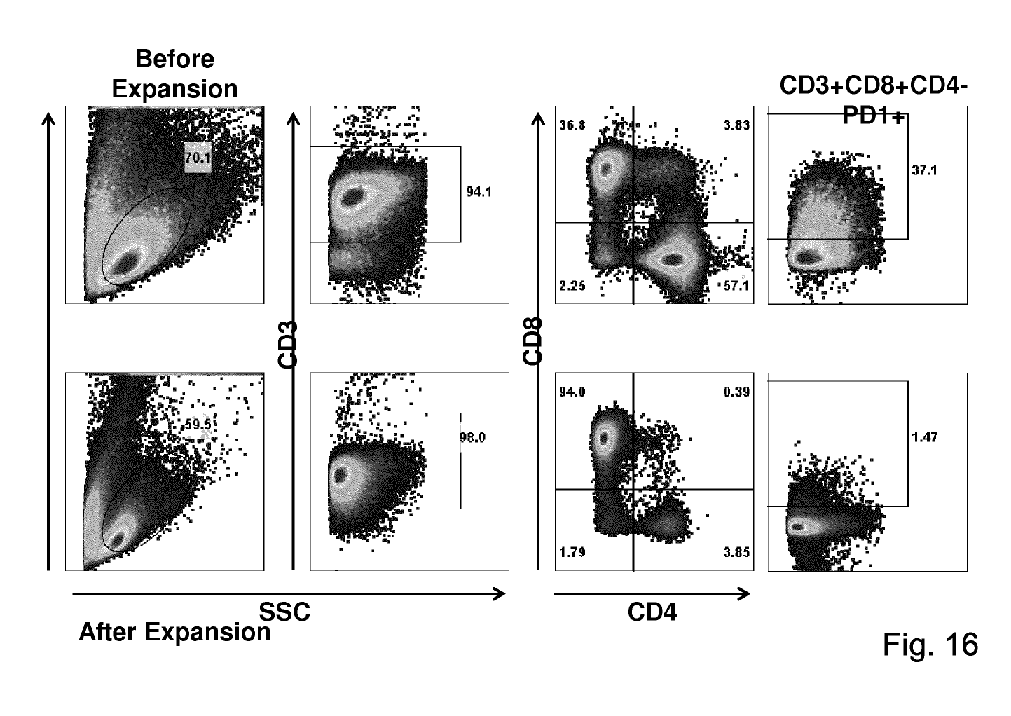

FIG. 18 shows a flow cytometric analysis of PBMCs prior to IL-2/IL-15/IL-21 driven expansion in the presence of the tumor-associated antigen NY-ESO-1.

First, CD3+ T-cells are gated, then the CD3+ T-cells are gated on CD4+ and CD8+ T-cells.

FIG. 19 shows a flow cytometric analysis of PBMCs prior to IL-2/IL-15/IL-21 driven expansion in the presence of the tumor-associated antigen NY-ESO-1. First, CD3+ T-cells are gated, then the CD3+ T-cells are gated on CD4+ and CD8+ T-cells.

FIG. 20 shows images of tumor-infiltrating cultured in-vitro with cytokines IL-2, IL-15, and IL-21 lymphocyte culture of the one week of incubation.

FIG. 21 shows an overview of the functional scheme of the cytotoxicity assay of expanded lymphocytes against autologous tumor cells using radioactivity (Cr 51) labelling and release.

FIG. 22 shows the results of a flow cytometric analysis of lymphocytes expanded from TIL obtained from patients with Glioblastoma. FIG. 3(A) shows the distribution of T-cell phenotypes in expanded TIL from 16 TIL into specific phenotypes: precursor (CD45RA+ CCR7+), central memory (CD45RA−CCR7+), peripheral memory (CD45RA−CCR7−), and differentiated effector (CD45RA+ CCR7−) T-cells separately for base phenotypes CD8+ (left panel), CD4+ (right panel) and double negative T-cells (right panel). The individual data points represent the percentage of the specific phenotype based on the base phenotype. The data shows that IL-2, IL-15 and IL-21 expand TIL with a long-term memory phenotype, as well as T-cell precursors—that may provide long-term immune protection.

FIG. 22(B) shows the expression of T-cell activation and exhaustion markers. The results are grouped like in (A) according to the base phenotype CD8+ (left panel), CD4+ (right panel) and double negative T-cells (right panel). The individual data points represent the percentage of cells expressing the marker indicated on the X-axis based on the base phenotype. CD117 (c-kit) is a ‘stem-cell’ associated marker and designates T-cells with a long-term memory, CD107a represents a marker for recent T-cell degranulation. The data show that TIL expanded in IL-2, IL-15 and IL-21 express markers (e.g. c-kit) that enables them for long-term immune cell memory and immune-surveillance.

FIG. 23 shows the results of a flow cytometric analysis of lymphocytes (TIL) expanded from tumor tissue of patients with pancreas cancer. The left panel shows the distribution of CD4+ T-cells into precursor (CD45RA+ CCR7+), central memory (CD45RA−CCR7+), peripheral memory (CD45RA−CCR7−), and differentiated effector (CD45RA+ CCR7−). The right panel the distribution of CD 8+ cells. The data shows that IL-2, IL-15 and IL-21 expand TIL with a long-term memory phenotype, as well as T-cell precursors—that may provide long-term immune protection.

FIG. 24 shows the results of a flow cytometric analysis of lymphocytes (TIL) expanded from tumor tissue of patients with pancreas cancer with respect to T-cell activation and exhaustion markers (4-1BB, LAG-3, TIM-3 et seq.). The results are grouped according to the CD4+/CD8+ phenotypes CD4+ (upper panel), CD 8+ (middle panel), DN (lower panel). The individual data points represent the percentage of cells expressing the marker indicated on the X-axis based on the base phenotype. The data show TIL that express a wide range of markers indicative for strong anti-tumor responses and recent antigen-exposure. The CD127 molecule (IL-7R) mediates strong T-cell survival factors.

FIG. 25 shows the TCR length distribution of T-cells expanded from tumor tissue of patients with pancreatic cancer determined by a PCR-based approach.

FIG. 26 shows the results of an intracellular cytokine production assay in CD4+, CD8 or DN T-cells in expanded lymphocytes from Glioblastoma lesions. The graphs in FIG. 7B show the percentage of T-cells producing the cytokines IFNγ and TNFα after stimulation. FIG. 12A shows maximal stimulation by PMA/ionomycin (positive control) and background by medium only. FIG. 7B shows the results of stimulation by synthetic peptides derived from tumor-associated antigens, i.e. the EGRvrIII, NY-ESO-1 or survivin. The data show that IL-2, IL-15 and IL-21 expanded TIL from patients with glioblastoma contain T-cells that react at low frequency to commonly shared tumor-associated antigens.

FIG. 27 shows the results of an intracellular cytokine production assay in CD4+, CD8 or DN T-cells in expanded lymphocytes from pancreatic cancer lesions. The graphs in FIG. 8A show the percentage of T-cells producing the cytokines IFNγ (upper panel) and TNFα (lower panel) after stimulation with tumor-associated antigens, i.e. Mesothelin, NY-ESO-1 or surviving in the CD4+ (left), CD8+ (middle) and DN subset (right). FIG. 8 B displays examples of a flow-cytometric analysis with NY-ESO-1 stimulation. T-cells gated on CD3+ and then on CD8+. are in the side scatter (SSC) versus IFNγ (upper box) or TNFα (lower box) production. The show that IL-2, IL-15 and IL-21 expanded TIL from patients with pancreatic cancer show a strong reactivity to a commonly shared tumor antigens, i.e. NY-ESO-1.

FIG. 28 shows the results of an intracellular cytokine production assay in CD4+, CD8 or DN T-cells in expanded lymphocytes from glioblastoma lesions after stimulation with autologous tumor cells. The graphs in FIG. 9A show the percentage of T-cells producing the cytokines IFNγ and TNFα for CD4+ left panel, CD8+ (middle panel) and DN T-cells (right panel) after stimulation with autologous tumor cells. FIG. 9 B displays examples of a flow-cytometric analysis of cells stimulated with autologous tumor cells. T-cells were gated on CD3+ and then on CD 4+ (upper box) or CD8+(lower box) are in the side scatter (SSC) versus IFNγ (upper box) or TNFα (lower box) production. The show that IL-2, IL-15 and IL-21 expanded TIL from patients with glioblastom show a strong reactivity to autologous tumor cells.

FIG. 29 shows the results of an intracellular cytokine production assay measuring TNFα production of lymphocytes expanded with the cytokine cocktail of IL-2, IL-15 and IL-21. The top panel shows the positive control (maximal stimulation). The middle panel shows results CD4+ gated expanded T-cells cytokine production in response to autologous tumor cells (left: all TIL, right: TIL gated on VB2+ T-cells). Lower panel: background production in the entire TIL population (left) and in the VB2+ TIL (right). The data show that preferentially expanded TCR VB families in IL-2, IL-15, IL-21 TIL (here: TCR VB2) are directed against autologous tumor cells.

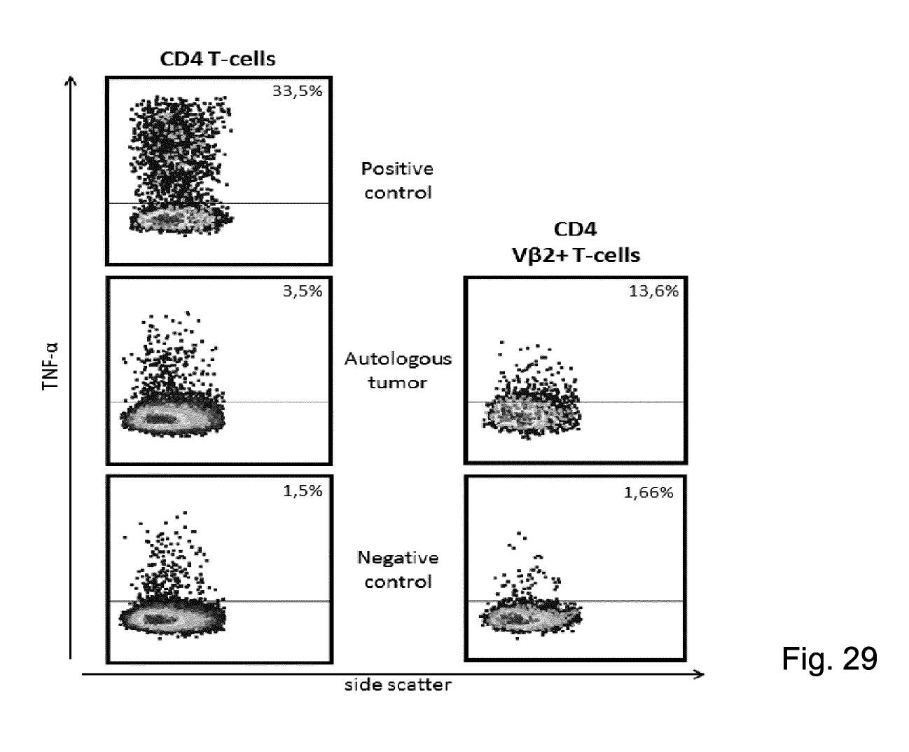

FIG. 30 shows the level of INFy in lymphocytes expanded from pancreatic tumor tissues after after stimulation. TIL+tumor stands for a stimulation of the expanded lymphocytes with autologous tumor cells. TIL+OKT3 stands for a stimulation of the lymphocytes with a CD3 antibody. W6/32 is an antibody blocking CD8+ TIL. Antibody L243 blocks CD4+ TIL. The data show that IL-2, IL-15 and IL-21 expanded TIL are specific against the patients autologous tumor.

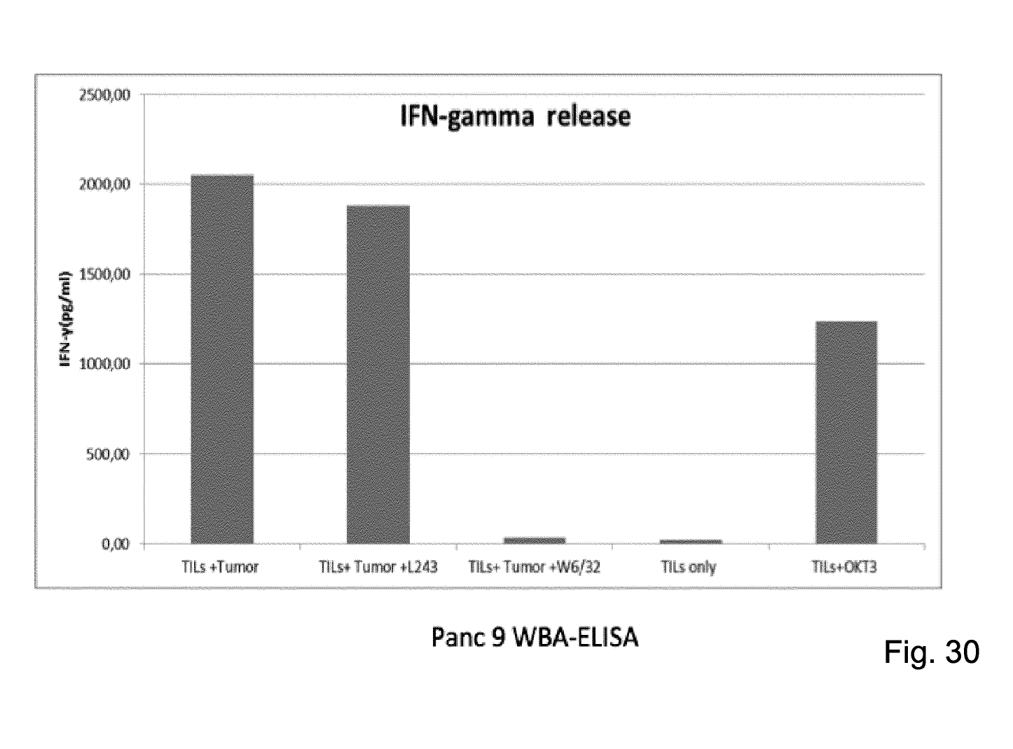

FIG. 31 shows the result of an analysis of the cytolytic response of expanded TIL from patients with glioblastoma against autologous tumor cells. The numbers on the X-axis represent the ratio of TIL to tumor cells. The percentage on the Y-axis represents the number of killed tumor cells after 4 h of treatment with expanded TIL measured by radioactivity release.

FIG. 32 shows the result of an analysis of the cytolytic response of expanded monoclonal T-cells and/or preferentially expanded TIL from patients with glioblastoma against autologous tumor cells. The numbers on the X-axis represent the ratio of TIL to tumor cells. The percentage on the Y-axis represents the number of killed tumor cells after 4 h of treatment with expanded TIL measured by radioactivity release.

FIG. 33 shows the result of an analysis of the cytolytic response of expanded TIL from patients with pancreatic cancer against autologous tumor cells. The numbers on the X-axis represent the ratio of TIL to tumor cells. The percentage on the Y-axis represents the number of killed tumor cells after 4 h of treatment with expanded TIL measured by radioactivity release. These IL-2, IL-15 and IL-21 expanded TIL showed a very focused TCR repertoire and show a strong cytotoxic response against the autologous tumor cells.

### DETAILED DESCRIPTION OF THE INVENTION

The inventors have found that a combination of the interleukins IL-2, IL-15 and IL-21 provides significant improvements for immunotherapy with lymphocytes. One major advantage is that the expansion and stimulation of lymphocytes derived from a patient with the composition of a combination of at least two types of cytokines selected from IL-2, IL-15 and IL-21 specifically favors the generation of lymphocytes, in particular T-cells, which are clinically relevant.

According to the invention “clinically relevant lymphocytes” are specific for and interact with clinically relevant antigens. There are three groups of clinically relevant lymphocytes, namely tumor-reactive lymphocytes, infectious disease reactive lymphocytes and autoimmune disease reactive lymphocytes.

“Clinically relevant lymphocytes” are also referred to as antigen-edited lymphocytes. The term clinically relevant is also used for subgroups of lymphocytes. Particularly preferred clinically relevant lymphocytes are clinically relevant T-cells or antigen-edited T-cells.

“Clinically relevant antigens” according to the invention are antigens involved in a disease. Accordingly, clinically relevant antigens can be tumor-associated antigens TAA, pathogen associated antigens (PAA) or autoantigens. Tumor-reactive lymphocytes are specific for and interact with TAAs. Infectious disease reactive lymphocytes are specific for and interact with PAAs and autoimmune disease reactive lymphocytes are specific for and interact with autoantigens.

According to the invention an “antigen” (Ag) is any structural substance which serves as a target for the receptors of an adaptive immune response, TCR or antibody, respectively. Antigens are in particular proteins, polysaccharides, lipids and substructures thereof such as peptides. Lipids and nucleic acids are in particular antigenic when combined with proteins or polysaccharides.

“Pathogen associated antigens” (PAA) refer to parts, such as capsules, cell walls, flagella and toxins of pathogens such as bacteria, viruses and other microorganisms.

“Autoantigens” are usually peptides, oligopeptides, polypeptides or complexes of proteins from an individual that are recognized by the immune system of the same individual. This effect usually leads to an autoimmune disease.

“Tumor associated antigens” or “TAA” according to the invention are antigens that are presented by MHC I or MHC II molecules or non-classical MHC molecules on the surface of tumor cells. As used herein TAA includes “tumor-specific antigens” which are found only on the surface of tumor cells, but not on the surface of normal cells.

With a combination of IL-2, IL-15 and IL-21 it is possible to specifically induce the proliferation of the clinically relevant lymphocytes in a body sample obtained from a patient as shown in the examples. The method according to the invention provides an easy protocol for the expansion of clinically relevant lymphocytes. It is in particular advantageous in comparison to the protocols in the state of the art as no dendritic cells are required. Moreover, the inventors could show that the resulting lymphocyte population after expansion with the cytokine cocktail of a combination of at least two types of cytokines selected from IL-2, IL-15 and IL-21 contains a composition of lymphocytes that is advantageous for clinical application. For example, the composition has a high percentage of TH1 helper T-cells and almost no T_{H2} helper T-cells. A further advantage is that there is no significant expansion of regulatory T-cells which might cause a suppression of the therapeutic action of the expanded lymphocyte population.

Thus, according to a first aspect the invention provides a composition for expanding lymphocytes comprising at least two types of cytokines selected from interleukin 2 (IL-2), interleukin 15 (IL-15) and interleukin 21 (IL-21).

IL-2, IL-15 and IL-21 are members of the cytokine family each of which has a four alpha helix bundle. IL-2 has key roles in key functions of the immune system, tolerance and immunity, primarily via its direct effects on T-cells. IL-2 induces T-cells proliferation and differentiation into effector and memory T-cells.

IL-15 is a cytokine that is structurally similar to IL-2. Like IL-2, IL-15 binds to and signals through a complex composed of the IL-2/IL-15 receptor beta chain. IL-15 induces a T-cell activation and proliferation in particular of CD8+ T-cells (30) and also provides survival signals to maintain memory cells in the absence of antigens, favored CD8+ T-cells and activates monocytes. IL-15 appears to drive rather proliferation of immune effector T-cells, along with the protection from inhibition of tumor-associated immunosuppression (31).

IL-21 is a cytokine that has potent regulatory effects on cells of the immune system, including natural killer (NK) cells and cytotoxic T-cells. IL-21 enriches central memory type T-cells with a CD28+ CD127hi CD45RO+ phenotype and enhances the cytotoxity of cytotoxic T-cells. IL-21 may keep T-cells in their early phase of differentiation and maturation (35).

According to the invention the composition of a combination of at least two types of cytokines selected from IL-2, IL-15 and IL-21 is also referred to as “the cytokine cocktail”.

As used herein, “interleukin 2” or “IL-2” refers to human IL-2 as defined by SEQ ID NO: 1 and functional equivalents thereof. Functional equivalents of IL-2 include relevant substructures or fusion proteins of IL-2 that remain the functions of IL-2. Accordingly, the definition IL-2 comprises any protein with a sequence identity to SEQ ID NO: 1 of at least 80%, preferably at least 90%, more preferably at least 95%, most preferably at least 98%. Recombinant human IL-2 produced in E. coli as a single, non-glycosylated polypeptide chain with 134 amino acids and having a molecular mass of 15 kDa is commercially available in lyophilized form from Prospec as CYT-209.

As used herein, “interleukin 15” or “IL-15” refer to human IL-15 and functional equivalents thereof. Functional equivalents of IL-15 include relevant substructures or fusion proteins of IL-15 that remain the functions of IL-15. Accordingly the definition IL-15 comprises any protein with a sequence identity to SEQ ID NO: 2 of at least 80%, preferably at least 90%, more preferably at least 95%, most preferably at least 98%. Recombinant human IL-15 produced in E. coli as a single, non-glycosylated polypeptide chain with 114 amino acids (and an N-terminal Methionine) and having a molecular mass of 12.8 kDa is commercially available in lyophilized form from Prospec as CYT-230.

As used herein, “interleukin 21” or “IL-21” refer to human IL-21 and functional equivalents thereof. Functional equivalents of IL-21 included relevant substructures or fusion proteins of IL-21 that remain the functions of IL-21. Accordingly the definition IL-21 comprises any protein with a sequence identity to SEQ ID NO: 3 of at least 80%, preferably at least 90%, more preferably at least 95%, most preferably at least 98%. Recombinant human IL-21 produced in E. coli as a single, non-glycosylated polypeptide chain with 132 amino acids and having a molecular mass of 15 kDa is commercially available in lyophilized form from Prospec as CYT-408.

A “peptide” as used herein may be composed of any number of amino acids of any type, preferably naturally occurring amino acids, which, preferably, are linked by peptide bonds. In particular, a peptide comprises at least 3 amino acids, preferably at least 5, at least 7, at least 9, at least 12, or at least 15 amino acids. Furthermore, there is no upper limit for the length of a peptide. However, preferably, a peptide according to the invention does not exceed a length of 500 amino acids, more preferably it does not exceed a length of 300 amino acids; even more preferably it is not longer than 250 amino acids.

Thus, the term “peptide” includes “oligopeptides”, which usually refer to peptides with a length of 2 to 10 amino acids, and “polypeptides” which usually refer to peptides with a length of more than 10 amino acids.

The term “protein” refers to a peptide with at least 60, at least 80, preferably at least 100 amino acids.

The term “fusion protein” according to the invention relates to proteins created through the joining of two or more genes, cDNAs or sequences that originally coded for separate proteins/peptides. The genes may be naturally occurring in the same organism or different organisms or may synthetic polynucleotides.

The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et a/., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using thenobrief option) is used as the percent identity and is calculated as follows:

(Identical Residues×100)/(Length of Alignment−Total Number of Gaps in Alignment)

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim, except for impurities ordinarily associated therewith. When the phrase “consists of” appears in a clause of the body of a claim, rather than immediately following the preamble, it limits only the element set forth in that clause; other elements are not excluded from the claim as a whole. The transitional phrase “consisting essentially of” limits the scope of a claim to the specified materials or steps “and those that do not materially affect the basic and novel characteristic(s)” of the claimed invention. “A ‘consisting essentially of’ claim occupies a middle ground between closed claims that are written in a ‘consisting of’ format and fully open claims that are drafted in a ‘comprising’ format.”

“Expansion” or “clonal expansion” as used herein means production of daughter cells all arising originally from a single cell. In a clonal expansion of lymphocytes, all progeny share the same antigen specificity.

According to one embodiment of the invention the composition according to the first aspect comprises two or three types of cytokines. Further cytokines may interfere with the expansion results provided by the composition according to the invention.

Alternatively, other cytokines used in addition to a combination of IL-2, IL-15 and IL-21 may positively influence the lymphocyte population. Thus, the composition of the first aspect of the invention may contain more cytokines in addition to IL-2, IL-15 and IL-21. Examples are IL-1beta, IL-4, GM-CSF, IL-12, IL-8, IL-17, TNFα, IL-32. IL-1β is involved in priming, the differentiation into effector B-cells or T-cells upon first contact with the specific antigen. IL-4 and GM-CSF are involved in dendritic cell stimulation and/or priming. IL-12 is involved in T_{H1} responses. IL-18 stimulates γδ-T-cells. IL-17 and TNFα act pro-inflammatory. IL-32 also acts pro-inflammatory favoring long-term protective immune responses.

According to one embodiment of the first aspect the composition comprises IL-2 and IL-15. The composition may also comprise IL-2 and IL-21. Alternatively, the composition may comprise IL-15 and IL-21. Although, already two of the cytokines IL-2, IL-15 and IL-21 may be enough to obtain a population of clinically relevant lymphocytes it is preferred to use all three cytokines in the composition.

According to a further embodiment, the composition of first aspect is in liquid form. In particular the composition is a cell culture medium. According to the invention any known cell culture medium is possible. Non-limiting examples of cell culture media are a synthetic medium, a medium derived from serum, plasma or whole blood or any combination thereof.

According to a further embodiment, the concentration of IL-2 in the liquid composition is in the range of from 10 to 6000 U/ml. The International Unit (U) is the standard measure for an amount or IL-2. It is determined by its ability to induce the proliferation of CTLL-2 cells. A concentration below 10 U/ml is too low to achieve any significant effect. A concentration above 6000 U/ml might have a cytotoxic effect. The concentration of IL-2 is preferably in the range from 500 to 2000 U/ml. More preferably the concentration of IL-2 is in the range from 800 to 1100 U/ml. As shown in the examples optimal results were achieved with a concentration of about 1000 U/ml.

According to a further embodiment of the first aspect, the concentration of IL-15 is in the range of 0.1 to 100 ng/ml. The concentration range follows the same rational as for IL-2. A concentration below 0.1 ng/ml is believed not to have any significant effect on the cells. A concentration above 100 ng/ml might have a cytotoxic effect. Preferably, the concentration of IL-15 is in the range from 2 to 50 ng/ml, more preferably in the range from 5 to 20 ng/ml. The most preferred concentration is about 10 ng/ml.

In a further embodiment the concentration of IL-21 is in the range from 0.1 ng/ml, preferably in the range from 2 to 50 ng/ml, more preferably in the range from 5 to 20 ng/ml.

It is to be understood that according to the invention any of these concentration ranges of one of the cytokines can be combined with any of concentration ranges of the other cytokines.

According to one embodiment the combination comprises a mixture of IL-15 and IL-21. The mixture comprises preferably each of IL-15 and IL-21 in the range form 10 to 100 ng/ml. IL-15 and IL-21 may provides synergistic effects, particularly on lymphocyte subsets in precursor, memory and effector populations.

According to one embodiment of the first aspect the combination comprises IL-2 in a concentration of 800 to 1000 U/ml and IL-15 and IL-21 in a concentration of 5 to 20 ng/ml. According to another embodiment the composition comprises IL-2 in a concentration of about 1000 U/ml and IL-15 and IL-21 in a concentration of about 10 ng/ml.

The composition of IL-2, IL-15 and IL-21 is in particular beneficial for promoting the expansion of clinically relevant lymphocytes in a composition of lymphocytes, in particular a patient's sample. As shown in the examples the inventors have developed a method for preparing specifically clinically relevant lymphocytes from a patient's sample.

The composition of IL-2, IL-15 and IL-21 is in particular beneficial for promoting the expansion of clinically relevant lymphocytes in a composition of lymphocytes, in particular a patient's sample. As shown in the examples the inventors have developed a method decreasing the frequency of PD1 and LAG3+T-cells, where PD1 and/or LAG3 expression serves as a marker for T-cell exhaustion and not as marker for antigen-experienced T-cells.

The composition of IL-2, IL-15 and IL-21 is in particular beneficial for promoting the expansion of clinically relevant lymphocytes in a composition of lymphocytes, in particular a patient's sample. As shown in the examples the inventors have developed a method increasing the frequency of 4-1BB expression as a marker for antigen-experienced T-cells.

Thus, according to a second aspect the invention provides a method of preparing a population of clinically relevant lymphocytes comprising the steps of
obtaining a body sample from a mammal in particular a tissue sample or body liquid sample, comprising at least one lymphocyte and optionally separating the cells in the body sample,
culturing the body sample in-vitro to expand and/or stimulate lymphocytes in the sample wherein the culturing comprises using at least two types of cytokines selected from IL-2, IL-15 and IL-21,
and optionally determining the presence of clinically relevant lymphocyte in the cultured sample.

The at least two types of cytokines selected from IL-2, IL-15 and IL-21 are preferably used in the concentrations defined above. Preferably all three cytokines IL-2, IL-15 and IL-21 are used together.

As shown in the examples the method can be used for generating a population of tumor reactive lymphocytes, autoimmune disease reactive lymphocytes or infectious disease reactive lymphocytes. Preferably, the lymphocyte population generated by the method of the second aspect is a population of tumor reactive lymphocytes.

The lymphocytes in general will comprise a variety of different lymphocytes.

Among these lymphocytes may be lymphocytes present that have the right receptor to interact with a clinically relevant antigen, in particular a tumor associated antigen, an infectious disease associated antigen or an autoimmune disease associated antigen. With the method according to the invention this clinically relevant lymphocyte in particular is strongly expanded. However, other lymphocytes that do not have the specificity for the clinically relevant antigen also are expanded in the method according to the invention.

Thus, the outcome of culturing the cells from the body sample with the composition comprising IL-2, IL-15 and/or IL-21 leads to the formation of a population of lymphocytes which includes the population of clinically relevant lymphocytes. The determination of the presence of clinically relevant lymphocytes in the cultured sample is a possible but not necessary step of the method according to the second aspect of the invention. The steps useful to verify the expanded lymphocytes population indeed can be used as a therapeutic.

The body sample can be taken from any part of the body that contains lymphocytes. Examples of body samples are peripheral blood, cord blood, bone marrow, lymph nodes, liver pleural effusion, thorax, abdominal cavity, synvial fluid, peritoneum, retroperitoneal space, thymus, and tumor.

A lymphocyte sample derived from tumor is also referred as tumor infiltrating lymphocytes (TIL). “TIL” as used herein is short for “tumor infiltrating lymphocytes”. TIL is any kind of lymphocyte that is located in, on or around a tumor. TIL is any kind of lymphocyte that is located in and around a tumor.

Due to their localization in the tumor, the TIL may have experienced tumor-associated antigens. Accordingly, clinically relevant lymphocytes, in particular tumor reactive lymphocytes can be expanded with the method according to the invention without expansion antigen.

A sample from peripheral blood is also referred to peripheral blood mononuclear cells (PBMCs). Depending on the type of disease different body samples may be preferred.

As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

According to one embodiment of the second aspect the mammal from which the body sample is obtained is a human. The mammal may have a tumor disease, or be at risk of developing a tumor disease. The risk of developing a tumor disease includes a high risk, a moderated risk and a low risk. A mammal in such a pre-malignant condition for example has a premalignant lesion which is morphologically atypical tissue which appears abnormal under microscopic examination and in which cancer is more likely to occur as in its apparently normal counterpart.

Moreover, the mammal may have an infectious disease or be at risk of developing an infectious disease. The risk of developing an infectious disease includes a high risk, a moderated risk and a low risk. The mammal may also have an autoimmune disease or be at risk of developing and autoimmune disease. The risk of developing an autoimmune disease includes a high risk, a moderated risk and a low risk. For instance, a high risk to develop intracellular infections (CMV, EBV, TB, HPV) upon certain genetic mutations of the host (IFNγ receptor defects, or acquired antibodies directed against cytokines, e.g. IL-12 or IFNγ). An intermediate risk would be immune-suppression using corticosteroids or treatment of patients with anti-TNFα or TNFα-receptor directed reagents. A low risk could be the co-infection with other pathogens or the temporary decrease of immune-competence in the course of major surgery. Similar examples are different clinical presentations—in association with genetic markers—of Multiple Sclerosis, rare neurological diseases, e.g. narcolepsy, Rheumatoid Arthritis, as well as chronic autoimmune-diseases associated with the gastrointestinal system.

If the mammal has a tumor disease or is at high risk of developing a tumor disease preferred body sample are peripheral blood or the tumor itself. As shown in the examples lymphocytes from peripheral and tumor can be treated with a method according to the invention to develop strong antitumor properties. If the disease is an autoimmune disease the preferred body sample is peripheral blood. Moreover, when the disease is an infectious disease the preferred body sample is also peripheral blood. As shown in the examples clinically relevant lymphocytes can be expanded from peripheral blood in these cases. Without being bound to theory it is assumed that the peripheral blood contains lymphocytes that have been in contact with the clinically relevant antigen, e.g. on the tumor or the infection. Culturing of the body sample in vitro to expand and/or stimulate lymphocytes may comprise one or more sub-steps. Accordingly, in one embodiment the in vitro culturing comprises a first expansion step comprising a incubation in culture medium comprising IL-2, IL-15 and IL-21 until lymphocytes become detectable.

“Detectable” according to the invention means that the lymphocytes, for example, become visible, in particular by microscopy. Lymphocytes usually become detectable using standard light microscopy upon reaching a concentration of 5×10³ cells/ml.

The detection of the lymphocytes may include any method known in the art that is eligible to detect the presence of lymphocytes above a certain threshold. The first expansion step has the purpose of gently inducing cell proliferation together with a stimulation of the cells by the cytokine cocktail.

The time of incubation of the first expansion step is in the range from 6 hours to 180 days. The large range of incubation time is first of all due to the fact that samples from different donors may behave very differently. Also it was shown that the lymphocytes from different body samples have very different growth rates. For example lymphocytes derived directly from the tumor of a glioblastoma or a pancreas cancer grow very differently. From pancreas cancer derived lymphocytes are already detectable within two to five days. Lymphocytes derived from glioblastoma are only detectable after one to two weeks. Accordingly lymphocytes from other body samples may take even longer to become detectable.

Preferably, the incubation time of the first expansion step is in the range from 4 days to 10 days. It was shown that with peripheral blood cells, incubation times about 7 days are particularly beneficial for the outcome of other expansion. However, as mentioned above, depending on the sample an expansion of only 4 days may be enough or on the other hand about 10 days or more may be necessary. Due to the good results with PBMCs shown in the examples an incubation time in the range from 6 to 8 days, in particular about seven days, is preferred.

According to one embodiment of the invention the culture medium of the first expansion step comprises at least one expansion antigen. The expansion antigen is a known clinically relevant antigen or a fragment, a mutant or a variant thereof. As used herein a “mutant” is defined as an amino acid sequence that differs from the reference sequence by an insertion, deletion or replacement of at least one amino acid. The expansion antigen is preferably selected from TAA, PAA and autoantigen.

Preferably the method according to the invention comprises multiple copies of an expansion antigen. An increased number of copies of an expansion antigen leads to increased expansion rate of a clinically relevant lymphocyte, in particular T-cells.

In one embodiment of the invention the culture medium of the first expansion step comprises multiple expansion antigens. Preferably, the multiple expansion antigens include a known clinically relevant antigen and a one or more mutants of the clinically relevant antigen. Instead of the antigen itself also the MHC class I/peptide presenting the antigen, in particular peptide can be mutated. The use of one or more wild type, variant or mutants of the clinically relevant antigen or MHC class I molecules as expansion antigens leads to a diverse set of lymphocytes, in particular T-cells reactive against the nominal clinically relevant antigen.

According to a preferred embodiment of the method according to the second aspect, the body sample is tumor and no expansion antigen is used in culturing.

The tumor tissue is preferably washed twice before separating the cells in the tumor sample.

Another preferred embodiment of the invention, the culture medium of the first expansion step comprises multiple expansion antigens. By having multiple antigens the T-Cell product responds in a more diverse T-Cell receptor repertoire as demonstrated by VP-usage associated with the use of the stimulating antigen(s).

Another preferred embodiment of the invention the culture medium of the first expansion step comprises multiple expansion antigens. The stimulation of T-Cells from peripheral blood results in T-Cell recognizing diverse epitopes, defined by cytoxicity. When naïve T-Cells are selected from the blood and stimulated with such antigens, they result in diverse epitopes of the antigen being recognized.

This is in contrast to pre-stimulated T-Cells or T-Cells from the tumor, which tend to recognize a more limited number or standard number of epitopes.

The choice of expansion antigen is dependent on the disease to be treated. In particular, if the expanded clinically relevant lymphocyte population is to be used against the tumor diseases, the expansion antigen added to the first expansion step is a preferably TAA. Alternatively, if the disease to be treated is an infectious disease, the expansion antigen added in the first expansion step is a PAA. Moreover, if the population of clinically relevant lymphocytes is to be used for the treatment of an autoimmune disease, the expansion antigen is preferably an autoantigen.

It is believed that the expansion antigen in the first expansion step leads to a stimulation of clinically relevant lymphocytes within the cell mixture already at an early stage and thus together with the cytokine cocktail enhances the expansion of clinically relevant lymphocytes. This stimulation is also referred to as antigen activation or antigen editing.

The first expansion step may be followed by a second expansion step wherein the cells are incubated with feeder cells and/or an antibody against CD3 in addition to the at least two types of cytokines selected from IL-2, IL-15 and IL-21. An expansion with feeder cells and the antibody against CD3 has been described in the state of the art. It is believed that feeder cells lead to an improvement of cell growth. Feeder cells are irradiated cells that do not proliferate or proliferate only to a small extent. The feeder cells increase the number of cell contacts in the culture and additionally feed the proliferating and expanding cell culture. Feeder cells are preferably in irradiated PBMCs. Allogeneic feeder. are from a different organism as the mammal to be treated with the expanded clinically relevant lymphocytes. Autologous feeder cells are from the mammal to be treated.

The antibody against CD3 is preferably the antibody defined as OKT3. OKT3 is a murine monoclonal antibody of the immunoglobulin IgG2a isotype. The target of OKT3, CD3, is a multi-molecular complex found only on mature T-cells. An interaction between T-cells, OKT3 and monocytes causes T-cell activation in vitro.

Preferably, feeder cells are used in combination with CD3 and the cytokines IL-2, IL-15 and IL-21. According to one embodiment of the method according to the second aspect, the ratio of feeder cells to lymphocytes is in the range from 1:1 to 1:100. Preferably, the ration of feeder cells to lymphocytes is in the range from 1:2 to 1:50. As shown in the examples very low ratios of feeder cells are sufficient for a strong expansion of clinically relevant lymphocytes, in particular clinically relevant T-cells.

In the examples, a ratio of 1:10 is sufficient to support growth and expansion of lymphocytes. Accordingly it is believed that a value in the range from 1:5 to 1:20 would not lead to a different result. The low number of feeder cells has at least two advantages. First, there are less distracting cellular signals allowing more homogeneous and reliable expansion results. Secondly, fewer feeder cells lead to less exogenous material in the immunotherapy product obtained by the method, i.e. the population of the clinically relevant lymphocytes.

The second expansion step optionally also includes an expansion antigen in the culture medium. Preferably no clinically relevant antigen or fragment is added to the medium of the second expansion step.

According to a preferred embodiment of the second aspect of the invention the method comprises a refocusing step. The refocusing step comprises a culturing in culture medium comprising refocusing cells. Refocusing cells are cells from the mammal, in particular a human, from which the body sample is obtained. Accordingly, the refocusing cells are autologous cells that have been treated with at least one refocusing antigen. Any expansion antigen as defined herein can also be used as refocusing antigen. Preferably, in the method the one or more refocusing antigens are identical to the one or more expansion antigens.

Refocusing cells are cells that have been incubated with the refocusing antigen for at least 30 minutes, or more, for example at least one hour, at least two hours, at least five hours, or at least ten hours. After incubation with the refocusing the refocusing cells are irradiated with at least 40 Gy. Preferably the cells are irradiated with at least 45 Gy, or more, for example at least 50 Gy and particularly preferred with an intensity of 55 Gy. Due to this treatment, antigen-specific T-cells are more effectively expanded, recognize tumor, pathogens or auto-immune cells and may confer protection against cancer cells or pre-malignant lesions.

The time of the refocusing step is in the range from 1 to 6 days, preferably 1 to 3 days. Although the refocusing step can be rather short, it leads to significant improvement in the yield of expanded clinically relevant lymphocytes, in particular for clinically relevant lymphocytes expanded from peripheral blood.

Also the number of refocusing cells in comparison to the number of lymphocytes is rather low. In particular the ratio of refocusing cells to lymphocytes is in the range from 1:1 to 1:100. It is found that the best results are achieved if the refocusing step is performed right after the first expansion step and followed by the second expansion step. The sequence of culturing steps is in particular useful for generating a population of clinically relevant lymphocytes, in particular T-cells.

According to one embodiment of the method, the first expansion step comprises adding IL-2, IL-15 and IL-21 simultaneously to the cell culture. For this, it is possible to prepare a mixture of IL-2, IL-15 and IL-21 and add the same to the cell culture medium or IL-2, IL-15 and IL-21 are added separately but simultaneously to the cell culture medium. As shown in the examples, the simultaneous application of IL-2, IL-15 and IL-21 leads to a preferred lymphocyte composition of the expanded lymphocyte population.

In order to shift the composition of the expanded lymphocyte population it is possible in the method of the invention to first add only IL-21 to the cell culture medium in the first expansion step. After the addition of IL-21, IL-15 and IL-2 may be added simultaneously or sequentially. Preferably IL-15 is added secondly and IL-2 added last. In an alternative embodiment IL-15 is added as the first cytokine, followed by simultaneous addition of IL-2 and IL-21 or a sequential addition of IL-2 and IL-21. For example, IL-21 is added secondly and IL-2 is added last.

The culture medium of the first and/or second expansion step may comprise least one expansion antigen. The expansion antigen may for example be fragment of a known TAA. Possible TAAs for as expansion antigen are for example NY-ESO-1, tyrosinase tumor antigen, tyrosinase related protein (TRP)-1, TRP-2, VEGFR-2, and a member of the MAGE family of proteins, telomerase, p53, HER2/neu, mesothelin, carcinoembryonic, survivin, EGFRvIII, VEGF, CAMPATH 1-antigen, CD22, CA-125, Mucin-1, Alpha-1-getoprotein, PSMA.

The fragment of the TAA is in particular a peptide. Such peptide can be for example a peptide comprising at least eight continuous amino acids of an amino acid sequence that is at least 80% identical to the amino acid sequence of SEQ ID NO: 4, SEQ ID NO: 5, SEQ ID NO: 6, SEQ ID NO: 7.

SEQ ID NO: 4 is the amino acid sequence of the known tumor associated antigen NY-ESO-1. SEQ ID NO: 5 is the amino acid sequence of the known tumor associated antigen survivin. SEQ ID NO: 6 is the amino acid sequence of the known tumor associated antigen mesothelin. SEQ ID NO: 7 is the amino acid sequence of the tumor associated antigen EGFRvIII.

The expansion antigen may for example be fragment of a known PAA. Possible PAAs as expansion antigens are for example CMVpp65, or EBV (EBNA-3, EBNA-1), HPV-16/33 E6, E7 or L1. The fragment of the PAA is in particular a peptide. Such peptide can be for example a peptide comprising at least eight continuous amino acids of an amino acid sequence that is at least 80% identical to the amino acid sequence of SEQ ID NO: 8, SEQ ID NO: 9, SEQ ID NO: 10, or SEQ ID NO: 11, which are the amino acid sequence of the known pathogen associated antigens CMVpp65, EBNA-3, EBNA-1, and HPV-L1, respectively.

The expansion antigen may for example be fragment of a known PAA. Possible autoantigens as expansion antigens are for example PRDM2, UCHL3, INO80E, SLC12A6, and Reelin. The fragment of the PAA is in particular a peptide. Such peptide can be for example a peptide comprising at least eight continuous amino acids of an amino acid sequence that is at least 80% identical to the amino acid sequence of SEQ ID NO: 12, SEQ ID NO: 13, SEQ ID NO: 14, or SEQ ID NO: 15, which are the amino acid sequences of the autoantigens PRDM2, IN080E, UCHL3, and DNaseB, respectively.

Additional components such as further cytokines may be added during the culturing step in particular during the first or second expansion step or the refocusing step.

Moreover, a promoter compound may be added in the culturing step, in particular in the first or second expansion step or in the refocusing step. Preferably the promoter compound is added in the first expansion step. A promoter compound as used herein is a compound that in the expansion process causes an increase a specific subset of lymphocytes. A preferred promoter compound is Zoledronic acid. Zoledronic acid promotes the expansion of gamma delta T-cells. A further preferred promoter compound is promotes the expansion of B-cells

According to one embodiment in the culturing step, in particular in the first or second expansion step or in the refocusing step a co-stimulatory compound is added. A co-stimulatory compound is for example a ligand for CD28 mediating T-cell signals. Examples of ligands for CD28 mediating T-cell signals are members of the B7 superfamily,in particular B7-1 (CD80) and B7-2 (CD86).

According to one embodiment of the method according to the second aspect testing for the presence of clinically relevant lymphocytes in the expanded lymphocyte sample comprises using evaluation antigens.

For the testing the lymphocyte population is incubated with the evaluation antigens. An evaluation antigen may be an antigen under the definition of the expansion antigen. For example, a known clinically relevant antigen or fragment thereof is added as evaluation antigen to the culture medium of the lymphocyte population to stimulate the lymphocytes. After an incubation time a parameter indicative for the activation of clinically relevant lymphocytes is measured.

Preferably the evaluation antigen is added to the lymphocytes in a form bound to a MHC I complex. For example, evaluation antigens may be presented to the lymphocytes bound to MHC dextramers. MHC dextramers are fluorescently labeled MHC multimers bound to a dextrose backbone.

The use of multimeric MHC structures has the advantage that multiple copies of the antigen can be presented to a single lymphocyte thereby increasing the simulation by the evaluation antigen. Alternatively known clinically relevant antigens may be presented to the cultured sample as evaluation antigens in the form of cells expressing the clinically relevant antigen as a transgene. Additionally, it is possible to add at least partially genetically matched allogeneic cells that present clinically relevant antigens on the cell surface to the expanded lymphocytes.

The parameter indicative for the presence of clinically relevant lymphocytes can be for example the production of one or more cytokines, in particular IFN-γ or TNFα production. Further parameters indicative of the presence of clinically relevant lymphocytes are an increased cell proliferation, an increased cytotoxicity, increased cell signaling and/or intracellular phosphorylation. The determination of these parameters is known in the art and exemplified in the examples.

Preferably in addition to using evaluation antigens derived from known clinically relevant antigens it is also possible to test for clinically relevant lymphocytes specific for the mammal to be treated. For this, cells, in particular tumor cells, that are derived from the same mammal as the expanded lymphocytes are used for presenting the evaluation antigens. Using these autologous cells as evaluation presenting antigens the presence of clinically relevant lymphocytes specific for clinically relevant antigens that are not necessarily known clinically relevant antigens can be verified.

In one embodiment according to the second aspect of the invention the evaluation antigens presented to the cultured sample are in a form selected from cells, in particular tumor cells, derived from the same mammal as the cultured sample (autologous cells) at least partially genetically matched allogeneic cells, in particular tumor cells or cell expressing the clinically relevant antigens as a transgene.

According to a further embodiment the method of the testing for the presence of clinically relevant lymphocytes comprises contacting the lymphocytes with at least one evaluation antigen and determining a change in either one of cytokine production, in particular IFN-γ or TNFα production, cell proliferation, cytotoxicity, signaling and/or intracellular phosphorylation.

The testing of these parameters can be combined with flow cytometry and cell sorting. Accordingly, it is also possible to isolate the clinically relevant lymphocyte population, in particular the tumor reactive lymphocyte population from the expanded lymphocyte population. The isolated population of clinically relevant lymphocytes may further be cultured or directly used for immunotherapy.

The method of the second aspect of the invention leads to the formation of population of lymphocytes, which includes a population of clinically relevant lymphocytes. Clinically relevant lymphocytes included in the expanded lymphocyte population can be any type of lymphocyte.

Lymphocytes include B-cells, NK-cells and T-cells. According to one embodiment the clinically relevant lymphocyte is a B-cell. According to one embodiment the clinically relevant lymphocyte is an NK-cell.

According a third aspect, the invention provides a clinically relevant lymphocyte obtained by the method according to the second aspect wherein the clinically relevant lymphocyte is a T-cell, an NK-cell or a B-cell.

The T-cell is preferably selected from a helper T-cell (TH-cell or CD4⁺−T-cell), in particular a T_{H1}-cell, a cytotoxic T-cell (T_{C}-cell or CD8⁺⁻T-cell), in particular CD8⁺CXCR3⁺ T-cell, a memory T-cell, in particular a central memory T-cell (T_{CM}-cell), a stem cell memory T-cell (T_{SCM}-Cell) or peripheral memory cell (T_{PM}-cell), a gamma-delta T-cell (γδ-T-cell), a NK-T-cell, a Mucosal-associated invariant T-cell (MAIT), a double-negative T-cell (CD3⁺CD4⁻CD8⁻T-cell).

According to one embodiment the clinically relevant lymphocyte is a lymphocyte that expresses molecules that facilitate entry into tissues, in particular tumor or infected or inflamed tissue (e.g. CXCR3). According to a further embodiment the clinically relevant lymphocyte is a lymphocyte that is enriched for markers of any long-term memory, in particular CD117 and c-kit and cytolytic immune cell responses, in particular CD107a. As explained the expanded lymphocyte population not only contains clinically relevant lymphocytes but also other lymphocytes that do not recognize clinically relevant antigens.

It is believed that clinically relevant and other lymphocytes in the culture obtained by the method according to the second aspect participate in the therapeutic effect. According to one embodiment of the third aspect, the invention relates to a lymphocyte obtained by a method according to the second aspect that expresses molecules and cytokines promoting the formation of combination of lymphocytes useful for medical application, including immunotherapy. The combination of lymphocytes useful for medical application preferably comprises T-cell precursors, T_{CM}, T_{SCM} and/or T_{PM} cells.

According to one embodiment the lymphocyte obtained by the method according to the second aspect expresses molecules and cytokines that promote the expansion of clinically relevant lymphocytes. According to a further embodiment the lymphocyte obtained by the method according to the second aspect produces cytokines selected from IFN-γ, TNF-α, IL-2, IL-17 and any combination thereof. According to one embodiment the lymphocyte is a CD3+CD4-CD8−T-cell.

According to a fourth aspect the present invention provides a population of lymphocytes obtained by the second aspect of the invention comprising a population of clinically relevant lymphocytes.

The population of lymphocytes may consist of a population of clinically relevant lymphocytes. The population of clinically relevant lymphocytes may be monoclonal, oligoclonal or polyclonal.

In one embodiment the population of clinically relevant lymphocytes is polyclonal and responds to multiple antigens or to different epitopes of the same antigen. Preferably, the population of clinically relevant lymphocytes responds to different antigens.

Lymphocytes, in particular T-cells responding to multiple antigens may prevent the risk of recurrence of pre-malignant cells, tumor cells and pathogens, thereby decreasing the risk for immune escape.

The population of lymphocytes according to the fourth aspect has a composition of different lymphocyte phenotypes, in particular T-cell phenotypes that is beneficial for immunotherapy.

The population of lymphocytes has a low percentage of regulatory T-cells. Regulatory T-cells are known to suppress the therapeutic function of the population of lymphocytes. According to one embodiment of the fourth aspect in the population of lymphocytes the percentage of T_{reg} based on the total number of T-cells is below 5%, preferably below 3%.

Moreover, the majority of T_{H} cells in the population of lymphocytes comprise the T_{H1} phenotype. According to one embodiment of the invention the percentage of T_{H1} cells based on the total number of T_{H} cells is at least 10%, preferably at least 50%, more preferably at least 70%.

In the population of lymphocytes the percentage of the cytotoxic CD8+ T-cells is increased as could be shown that increased percentage of CXCR3+ cells.

According to one embodiment of the invention the percentage of CXCR3+ cells based on the total number of CD8+ T-cells is at least 10%, at least 50%, more preferably at least 70%.

In addition the population of lymphocytes may comprise an increased number of T-cells that have recently been in contact with its antigen as for example identified by the 4-1 BB+ phenotype. According to one embodiment of the fourth aspect the percentage of 4-1BB+ T-cells based on the total number of T-cells is at least 1%, preferably at least 2%, more preferably at least 2.5%. According to one embodiment the percentage of CD107+ cells based on the total number of T-cells is at least 1%, preferably at least 2%, more preferably at least 2.5%. CD117+ T-cells have been associated with long-term memory population.

The CXCR3+ phenotype CD8+ T-cells is further associated with invasiveness into tissues and thus maybe in particular beneficial for immunotherapy against cancer. Further the population of lymphocytes may comprise sufficient percentage of CD3+CD4−CD8− cells. These double negative T-cells are highly specific for the antigen target as they are dependent of the co-receptor CD4+ or CD8+ and produce inflammatory cytokines. Thus, they are cytotoxic.

According to one embodiment a population of lymphocytes comprises a percentage of CD3+CD4−CD8− cells based on the total number of T-cells of at least 1%, preferably at least 3%, more preferably at least 5%. According to a further embodiment the percentage of gamma delta T-cells based on the total number of T-cells in a population of lymphocytes is at least 1%, preferably at least 3%, more preferably at least 5%. Gamma delta T-cells have the effect that they recognize stressed cells, transformed cells, infected cells, e.g. CMV+target cells. Gamma delta T-cells cross-recognize as well virally and transformed cells.

According to one embodiment the population of lymphocytes comprises the following features:
the percentage of Treg based on the total number of T cells is below 5%, preferably below 3%;-
the percentage of T_{H1}-cells based on the total number of T_{H}-cells is at least 50%, preferably at least 70%, more preferably at least 80%,
the percentage of CXCR3+ T-cells based on the total number of CD8⁺ T-cells is at least 50%, preferably at least 70%, more preferably at least 80%,
the percentage of 4-1BB+ T-cells based on the total number of T-cells is at least 1%, preferably at least 2%, more preferably at least 2.5%,
the percentage of CD117+ T-cells based on the total number of T-cells is at least 1%, preferably at least 2%, more preferably at least 2.5%,
the percentage of CD3+CD4−CD8− cells based on the total number of T-cells is at least 1%, preferably at least 3%, more preferably at least 5%; and
the percentage of γδT-cells based on the total number of T-cells is at least 1%, preferably at least 3%, more preferably at least 5%.

According to one embodiment in the population of lymphocytes, the percentage of precursor T-cells (CD45RA+ CCR7+) based on the number of total T-cells is at least 1%, preferably at least 2%, more preferably at least 3%. Central memory T-cells were already shown to relate to success of a T-cell therapy.

The central memory cells produce several cytokines beneficial for therapy, provide a good memory response. However, the central memory cells do not infiltrate into tissue well. In one embodiment the percentage of peripheral memory cells (CD45RA− CCR7−) based on the total number of T-cells at least 2%, preferably at least 5%, more preferably at least 10%. Peripheral memory cells show high cytokine production and infiltrate well into tissue. Thus, these cells are in particular useful for immunotherapy against cancer.

Also terminally differentiated T-cells (CD45RA+ CCR7−) are able to enter into a tissue. Moreover, these cells show a production of therapeutically useful cytokines.

According to one embodiment the percentage of effector T-cells (CD45RA+ CCR7−) based on the total number of T-cells is at least 1%, preferably at least 3%, more preferably at least 5%. The percentage of precursor T-cells (CD45RA+ CCR7+) based on the total number of T-cells may be at least 1%. Preferably the percentage is at least 2%, more preferably at least 3%. Precursor T-cells show only cytokine production and barely enter in tissue. However, these cells may be reverted T-cells with a lifelong memory effect.

The population of lymphocytes according to the fifth aspect may be further characterized by an intracellular cytokine production after stimulation with a evaluation antigen having a value that is at least twofold of the standard deviation without evaluation antigen stimulation. Moreover, the stimulation with the evaluation antigen leads to a value of CD107a induction that is at least twofold of the standard deviation without evaluation antigen stimulation.

Due to these parameters the population of lymphocytes and/or the population of clinically relevant lymphocytes represent useful immunotherapy products for the treatment of cancers, infectious diseases and autoimmune diseases.

According to a fifth aspect the invention provides an immunotherapy for treating or preventing a tumor disease, an infectious disease or an autoimmune disease in a mammal comprising the steps generating a clinically relevant lymphocyte population according to the second aspect of the invention wherein the body sample is obtained from said mammal and administering the clinically relevant lymphocyte population to said mammal. The infectious disease may be any known infectious disease relating to any known pathogen.

The tumor disease according to the invention can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, mesentery cancer, pancreatic cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colon cancer, and hepatobiliary cancer. Preferred cancers are glioblastoma and pancreatic cancer.

In order to avoid immune effects against the clinically relevant lymphocytes, the cancer patients may “conditioned” before administering the T-cells. The conditioning has three purposes. First is to provide more space for the infused lymphocyte population, second to remove adverse effectors and third to increase production of growth factors that may favor the rapid expansion and survival of T-cells, i.e. autologous IL-7 and IL-15 production.

The clinically relevant lymphocytes can be mixed with a pharmaceutically acceptable carrier to form a pharmaceutical composition, which is also within the scope of the present disclosure. In one embodiment, the clinically relevant lymphocytes may be autologous to the subject, i.e., the clinically relevant lymphocytes are obtained from the subject in need of the treatment, and then administered to the same subject.

Administration of autologous cells to a subject may result in reduced rejection of the host cells as compared to administration of non-autologous cells. Alternatively, the host cells are allogeneic cells, i.e., the cells are obtained from a first subject and administered to a second subject that is different from the first subject but of the same species. For example, allogeneic clinically relevant lymphocytes may be derived from a human donor and administered to a human recipient who is different from the donor.

To practice the methods disclosed herein, an effective amount of the clinically relevant lymphocytes described herein, or compositions thereof, can be administered to a subject (e.g., a human cancer patient, a patient with an infectious disease, a patient with an autoimmune disease) in need of the treatment via a suitable route, such as, for example, intravenous administration. The cells may be introduced by injection, catheter, or the like. If desired, additional drugs (e.g., cytokines) may also be co-introduced or introduced sequentially. Any of the cells or compositions thereof may be administered to a subject in an effective amount. As used herein, an effective amount refers to the amount of the respective agent (e.g., the cells or compositions thereof) that upon administration confers a desirable therapeutic effect on the subject. Determination of whether an amount of the cells or compositions described herein achieved the desired therapeutic effect would be evident to one of skill in the art. For example, see references 2-5. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of the desired disease or disorder in the subject.

“Administering” refers to the physical introduction of a composition comprising a cell or pharmaceutical composition described herein to a subject, using any of the various methods and delivery systems known to those skilled in the art.

The administering of the population of tumor reactive lymphocytes preferably is an introdution into the patient either locally in proximity of a tumor or into the tumor. Alternatively the population of tumor reactive lymphocytes is administered into the blood circuit. Other clinically relevant lymphocytes are preferably added into the blood circuit.

According to an embodiment of the present invention, the subject is administered with a dose of clinically relevant lymphocytes comprising at least about, 4×10⁶, 4.5 ×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 10×10⁶, 12.5×10⁶, 15×10⁶, 20×10⁶, 25×10⁶, 30×10⁶, 35×10⁶, 40×10⁶, 45×10⁶, 50×10⁶, 60×10⁶, 70×10⁶, 80×10⁶, 90×10⁶ of cells per kilogram body weight. {please expand to other ranges as appropriate}

The immunotherapy according to the fifth aspect population of clinically relevant lymphocytes
brings about regression of cancer cells in the mammal;
interferes with the move from pre-malignant to malignant lesions;
brings about fast senescence of tumor cells or pre-malignant cells;
brings about removal of autoantigen-positive cells;
brings about killing, growth arrest or containment of pathogens;
interferes with cancer stem cells; and/or
induce growth arrest of cancer cells, or cells expressing auto-antigens.

According to a sixth aspect the invention provides a composition according to the first aspect of the invention for use in the medical treatment, in particular for treating and preventing an infectious disease, an autoimmune disease or a tumor disease.

According to one embodiment of the sixth aspect the use comprises a generation of a population of clinically relevant lymphocytes with a method according to the second aspect of the invention.

According to one embodiment of the composition according to the sixth aspect the use comprises an immunotherapy according to the fifth aspect of the invention. As dicussed above, the particular cytokines of the cytokine cocktail not necessarily have to be used simultaneously but may be added at a different time point.

According to a seventh aspect the invention provides the kit for use in medical treatment, in particular for treating or preventing an infectious disease, an autoimmune disease or a tumor disease, wherein the kit comprises IL-2, IL-15 and IL-21. The kit further optionally comprises at least one of a component that stimulates the TCR, in particular OKT3, costimulatory molecules, feeder cells and a peptide comprising the amino acid sequence of clinically relevant antigen. Preferably the kit comprises all of the mentioned components.

The invention is further defined by the following examples.

### EXAMPLES

### Example 1

### Lymphocytes Expansion Protocol using Peripheral Blood Mononuclear Cells

A) Materials and Equipment

### i) Equipment

24 well plate, (Becton, Dickinson (BD), REF 353504)
Sterile scalpel
Sterile tweezers
Medimachine (tissue homogenate machine), (BD, Cat:340588)
Medicon, 50μm, (BD, Cat:340591)
Filcons, 200 μm (BD, Cat:340613)
Laminar flow hood (Class 2 biosafety hood)
Low temperature freezer, −80 ° C.
Refrigerator
Centrifuge

### ii) Supplies

Gloves (latex)
Lab coat
Sterile centrifuge tubes, 15 mL.
Pipet tips
Pipette aid
Waste container
   iii) Reagents
RPMI 1640, (Gibco, REF 61870-044)
Cellgro, (CellGenix, Cat:20801-0100)
Pooled human AB serum (Innoative Research, IPLA-SerAB-13458).
Fetal Bovine Serum (Gibco, REF: 26140-079).
Anti-CD3 antibody (OKT3), (Biolegend, Cat:317304).
Human IL-2 (Prospec, Cat: CYT-209-b).
Human IL-15 (Prospec, Cat:cyt-230-b).
Human IL-21 (Prospec, Cat:cyt-408-b)
PEST (antibiotics)
Amphotericin

B) Procedure

Aphaeresis was performed on a healthy donor primed with NY-ESO-1. After separation of the blood components the product containing the leucocytes is separated from the rest. Cells are suspended in Cellgro comprising 5% pooled human AB serum, 1000 U/ml IL-2, 10 ng/ml IL-15 and 10 ng/ml IL-21. The medium in addition contained 10 pmol NY-ESO-1 peptide as expansion antigen. The concentration of cells from. The expansion of lymphocytes from peripheral blood contained the step of stimulation by irradiated autologous PBMCs pulsed with the antigen of interest. For this, autologous PBMCs were pulsed for two hours with 10 mM NY-ESO-1 peptide at RT, and then irradiated at 55 Gy. The pulsed irradiated autologous PBMCs were added to the lymphocyte culture on day 7 and re-suspended in fresh culture medium supplemented with 5% human pooled AB serum with cytokines in concentrations as defined above. On day 10 cells were expanded with Cellgro supplemented with 5% pooled human AB serum and cytokines. OKT3 antibody was added in a concentration of 30 ng/ml in the same culture medium described as above plus irradiated feeder cells (55Gy) at a 1:10 (feeder cell: T-cell ratio) the peptide and cytokines. On day 17 to 20 of culturing the cells are harvested for analysis or transferred to the patient.

### Example 2

### Lymphocytes Expansion Protocol using Peripheral Blood Mononuclear Cells

The procedure of Example 2 is identical to the procedure of Example 1 only that the donor was a patient who received vaccination with NY-ESO-1.

### Example 3

### Lymphocytes Expansion Protocol using Peripheral Blood Mononuclear Cells

Example 3 is identical to Example 1 only that the donor is a patient whose tumor already expressed the TAA NY-ESO-1.

### Example 4

### Gamma Delta T-Cells can Enriched in Expanded Lymphocytes with the Cytokines IL-2, IL-15 and IL-21

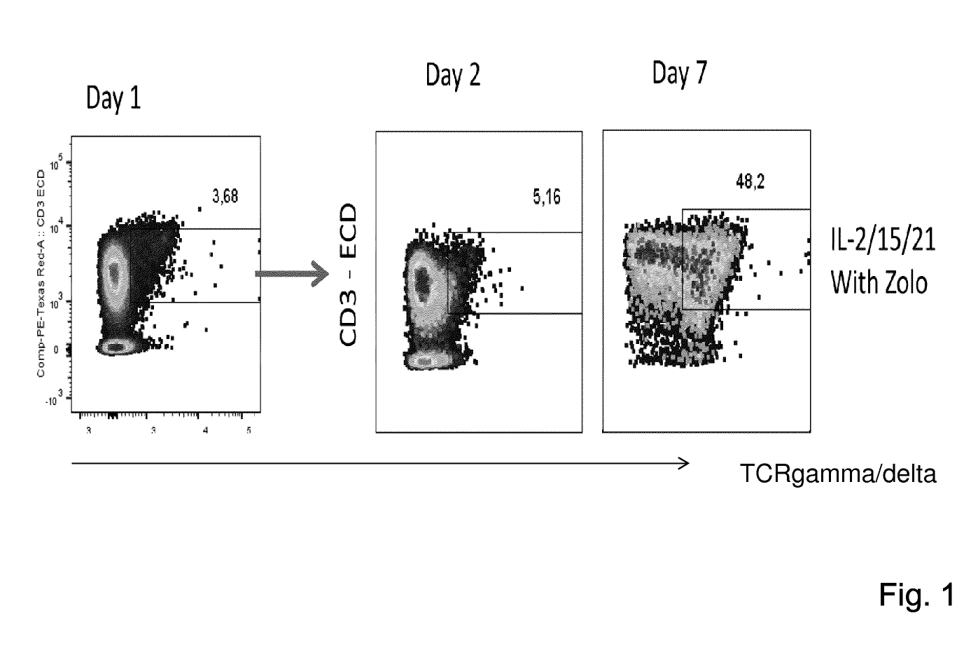

The expansion is carried out according to the protocol described in Example 1 with the difference that zoledronic acid was added to the cell culture in a concentration of 5 μMol. Zoledronic acid is known to promote the expansion of TCR gamma delta T-cells. As shown in FIG. 1 this experimental setup leads to a strong increase of TCR gamma delta T-cells in both frequency and absolute numbers. In FIG. 1 flow cytometry images of the expanded culture at different times are shown. Accordingly on the first day of expansion 3.68% of he cells carry the TCR gamma delta. There is already a slight increase on the second day. Strikingly, after 7 days of expansion almost 50% of T-cell are the γδ subset.

### Example 5

### Expansion of the Lymphocytes with the Cytokine Cocktail Induces Double Negative T-cells (CD3+CD4-CD8−).

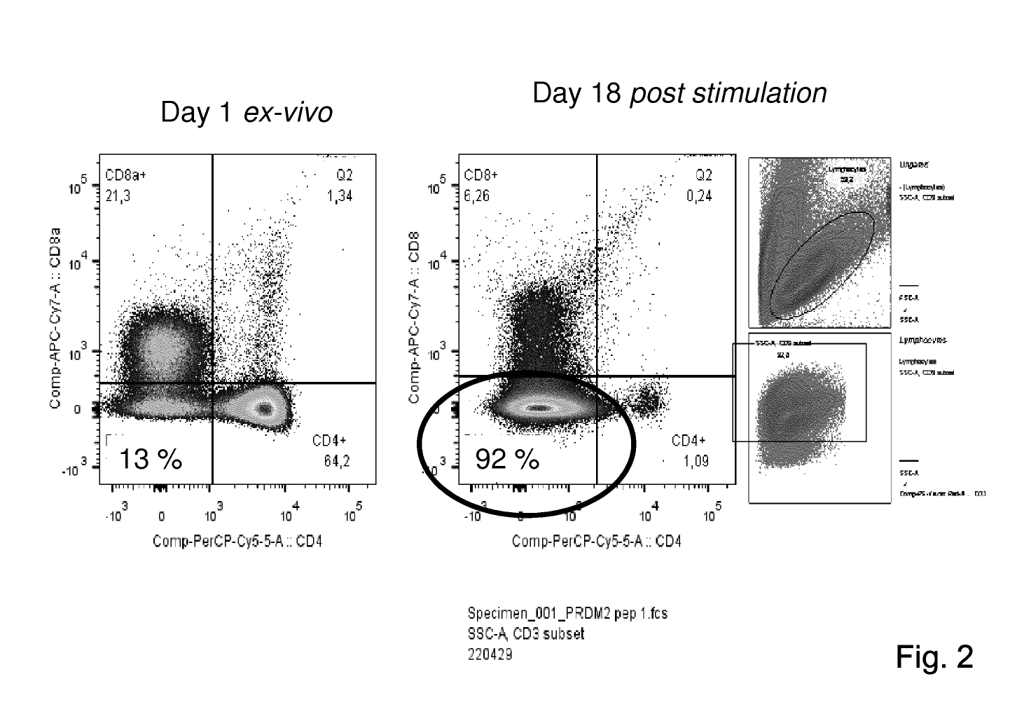

Lymphocytes were obtained from a patient with narcolepsy. The expansion was carried out as described in Example 1 but with the PRDM2 peptide as expansion antigen and stimulating. The cultured cells were tested for the T-cell phenotype on day 1 and day 18. FIG. 2 shows the flow cytometry results of these samples. In the results first the lymphocytes are filtered and then the CD3+ lymphocytes are filtered. These cells are then further analyzed for the presence of CD4 and CD8. From the panels it can be seen that after day 1 only 13% of the cells are in the double negative state. The majority of the cells are CD4+. After 18 days of expansion with the cytokine cocktail 92% of the cells are double negative cells (CD3+CD4−CD8−) T-cells.

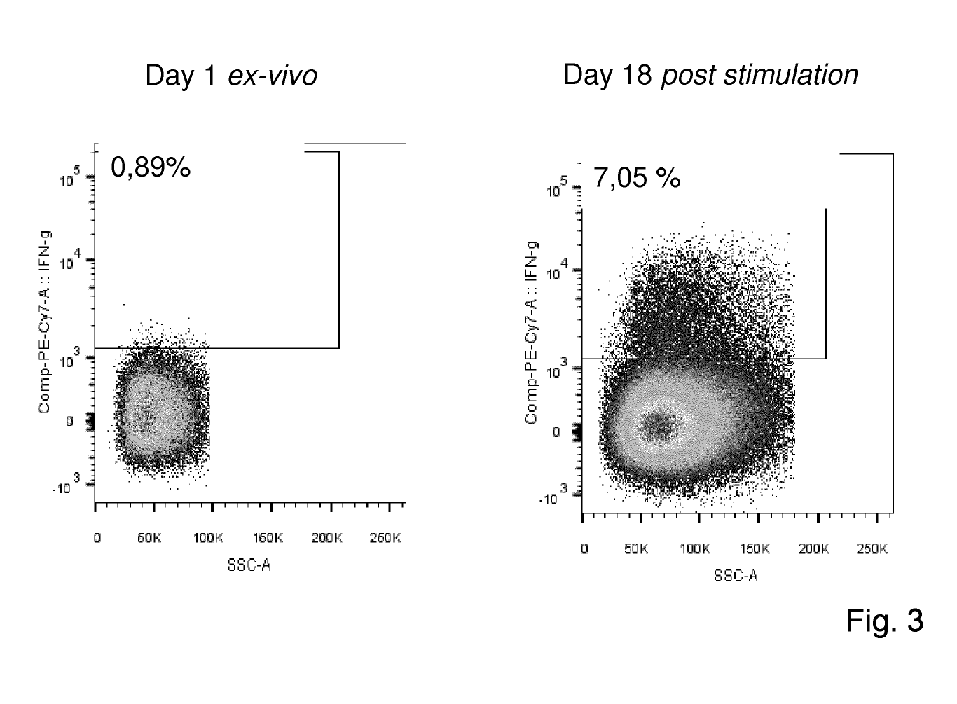

Furthermore, the induction of IFN-γ production of the expanded lymphocytes upon PRDM2 peptide stimulation is tested. As shown in FIG. 3 on day 1 only 0.89% of the cells produce IFN-γ upon PRDM2 stimulation. In contrast, the sample from day 18 shows a significantly increased population of cytokine producing PRDM2 specific T-cells. As most of the T-cells are in the CD3+CD4−CD8− state, this shows that the expansion protocol leads to the formation of clinically relevant double negative T-cells.

### Example 6

### Sorted Dextramer Cells Produce IFN-γ in Response to Autologous EBV-LCLs Loaded with Peptide and Protein Antigens

The expansion protocol according to Example 1 was carried out with blood from two different patients with narcolepsy again with PRDM2 as expansion peptide. In two different setups per patient either DNAseB or PRDM2 peptides were added to the cytokine cocktail in the culture medium. CD3+CD4−CD8− and conventional CD8+ T-cells that were MHC restricted and directed against PRDM2 or DNAseB were sorted by flow cytometry and tested whether they recognize naturally processed and presented epitopes. T-cells from the same patients were pulsed with peptides or with recombinant proteins and IFN-γ production was measured. The results of the experiment are summarized in Table 1:

TABLE 1

| | | | | | | | | T Cells + |
|---|---|---|---|---|---|---|---|---|
| | | Sorted Dextramers - | DNAseB | | PRDM2 | DNAseB | PRDM2 | Autologous |
| | Stimulus | % Frequency | WT | MUT | WT | Protein | | B Cells |
| Patient 1 | DNAseB | PRMD2 CD3+ DN- | 10.7 | 1.2 | 6.6 | 1.0 | 23.0 | 0.05 |
| | | 0.27% | | | | | | |
| | | DNAseB CD3+ DN- | 0.7 | 7.7 | 1.5 | 3.1 | 3.1 | 0.07 |
| | | 0.29% | | | | | | |
| | PRDM2 | PRMD2 CD3+ DN- | 0.0 | 0.0 | 2.3 | 0.0 | 0.0 | 0.10 |
| | | 0.25% | | | | | | |
| | | DNAseB CD3+ DN- | 5.3 | 0.6 | 0.4 | 1.8 | 2.7 | 0.0 |
| | | 0.45% | | | | | | |
| Patient 2 | DNAseB | PRMD2 CD8+- | 0.4 | 0.2 | 0.4 | 2.3 | 1.6 | 0.01 |
| | | 0.70% | | | | | | |
| | | DNAseB CD8+- | 1.1 | 0.9 | 0.4 | 2.5 | 1.1 | 0.04 |
| | | 0.26% | | | | | | |
| | PRDM2 | PRMD2 CD8+- | 0.9 | 25.8 | 10.7 | 1.3 | 6.5 | 0.0 |
| | | 0.28% | | | | | | |
| | | DNAseB CD8+- | 22.6 | 7.0 | 14.8 | 11.0 | 7.9 | 0.0 |
| | | 0.32% | | | | | | |

The measured values are the IFN-γ concentration in pg/ml. This experiment shows that the cytokine cocktail of IL-2, IL-15 and IL-21 in combination with a specific antigen is able to expand clinically relevant T-cells, in particular T-cells specific for the antigen in peripheral blood. The T-cells can preferentially reside in the so-called DN CD3+CD4−CD8− T-cell population and these T-cells are highly functionally active, they produce IFN-γ and recognize biologically and medically relevant targets.

### Example 7

### Analysis of Lymphocytes Expanded with the Cytokine Cocktail and Tumor Associated Antigens

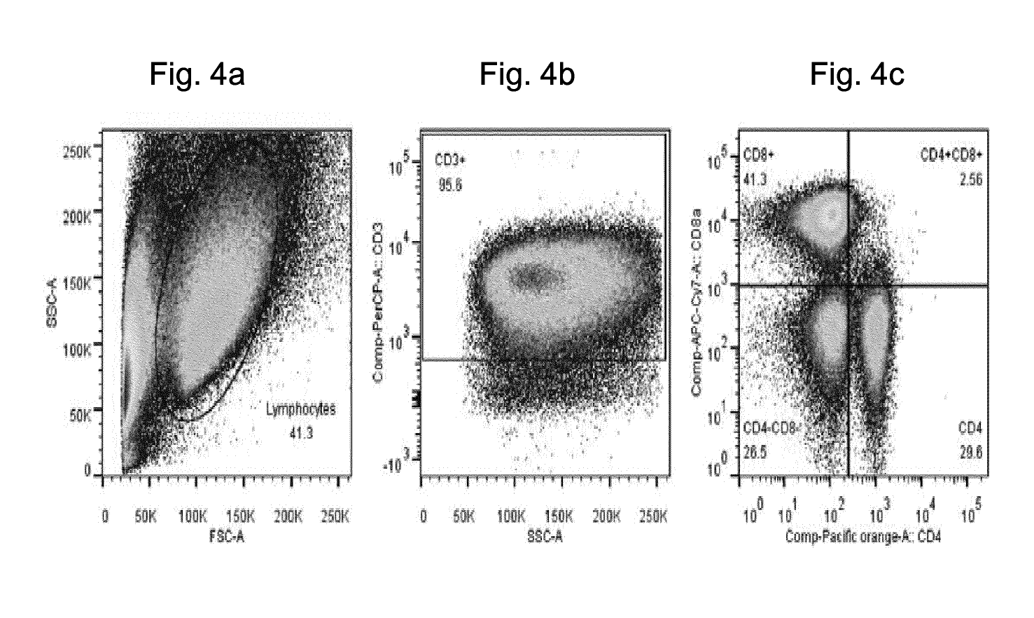

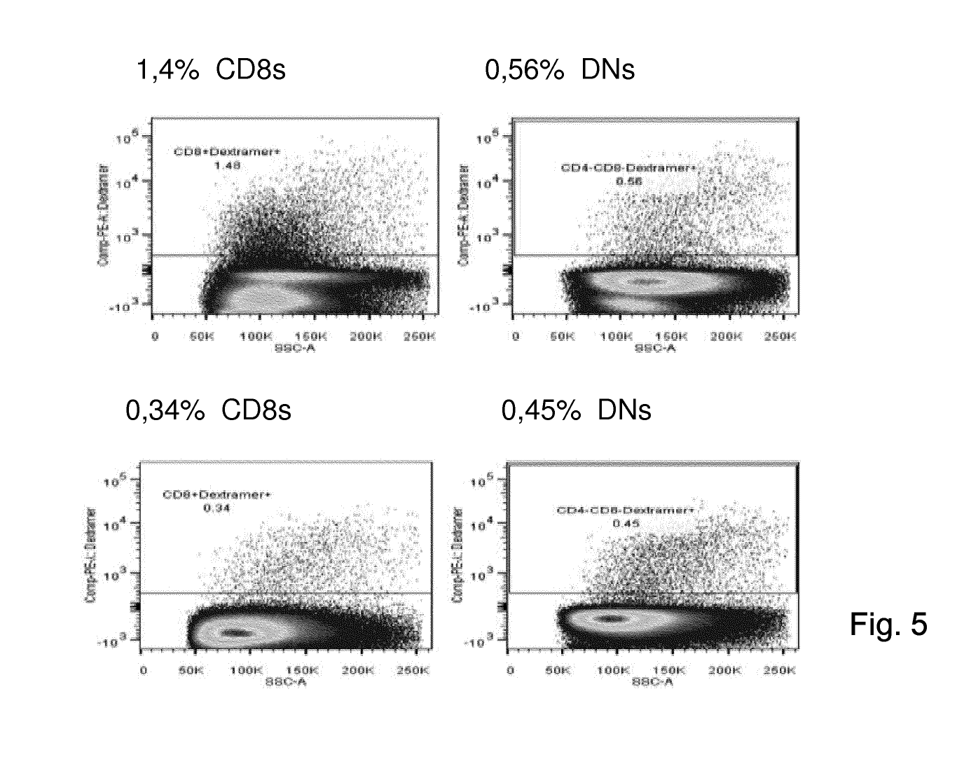

The expansion procedure was carried out as described in Example 1 with the exception that INO80E and UCHL3 were used instead of NY-ESO-1 as expansion antigen. After 18 days of expansion the cells were stimulated with either INO80E or UCHL3 analyzed by flow cytometry. The results of the flow cytometry are shown in FIGS. 4 and 5. FIG. 4A shows the signal of the cells separated by sideward and forward scattering. Lymphocytes are gated as shown by the black elliptic circle. The filtered lymphocytes are then again filtered for the presence of CD3. The CD3+ cells were further analyzed by a separation according to the presence or non-presence of CD8 and CD4. In this experiment 41% of the cells are CD8+, 29.6% CD4+ and 26.5% double negative. The double negative and CD8+ were then tested for IFN-γ production upon stimulation with the target antigens INO80E and UCHL3. Note that the T-cells are objectively measured using MHC class 1/peptide complexes. According to the results shown in FIG. 5A 1.4% of the CD8+ cells produce IFN-γ upon INO80E stimulation but also 0.56% of the double negative T-cells produce IFN-γ upon stimulation (see FIG. 5B.). Similar results are obtained for UCHL3 activation as shown in FIG. 5 0.34% of the CD8+ cells produce IFN-γ. Also 0.45% of the double negative cells produce IFN-γ (see FIG. 5D).

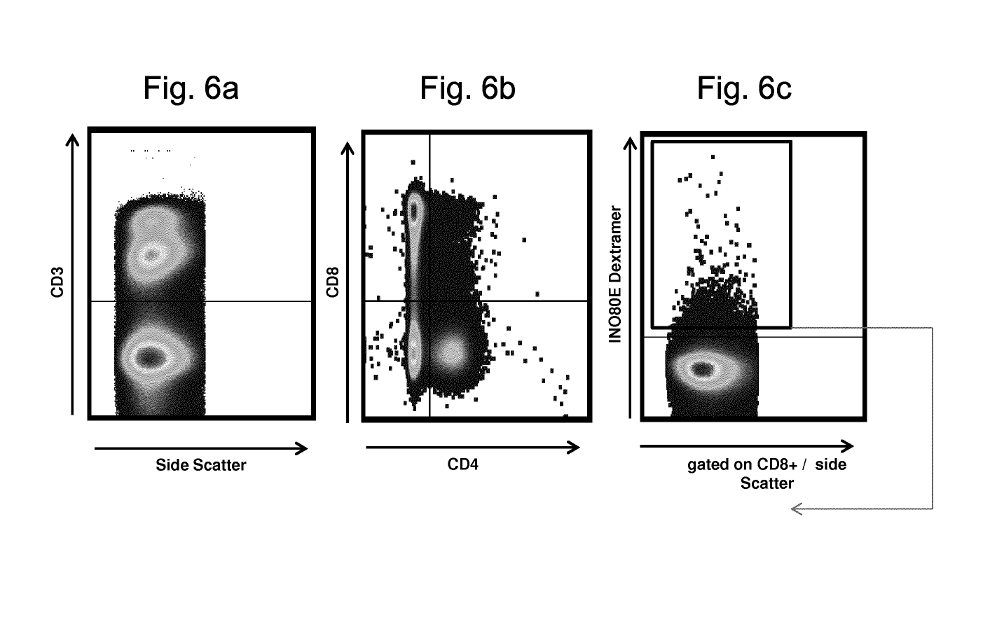

The cells stimulated with INO80E were further analyzed for the production of cytokines. FIG. 6A to 6C again shows the gating of CD8+ IFN-γ producing T-cells. The analysis of cells producing CD107a, CD127 (IL-7R) and CD117 are shown in FIG. 6D to 6F. Grey signals stand for the entire CD3+CD8+ T-cell population (irrespective of the antigen-specificity), black signals for CD3+CD8+ T-cells that are antigen-specific. The analysis shows that the TAA reactive CD8+ T-cells express CD107a, a marker associated with ccytotoxicity and CD127 (the II-7R receptor, mediating survival signal signals), but not CD117, a marker for T-cells with stem-cell like properties.

### Example 8

### Expansion of PBMCs with the Cytokine Cocktail and the CMVpp65 Peptide

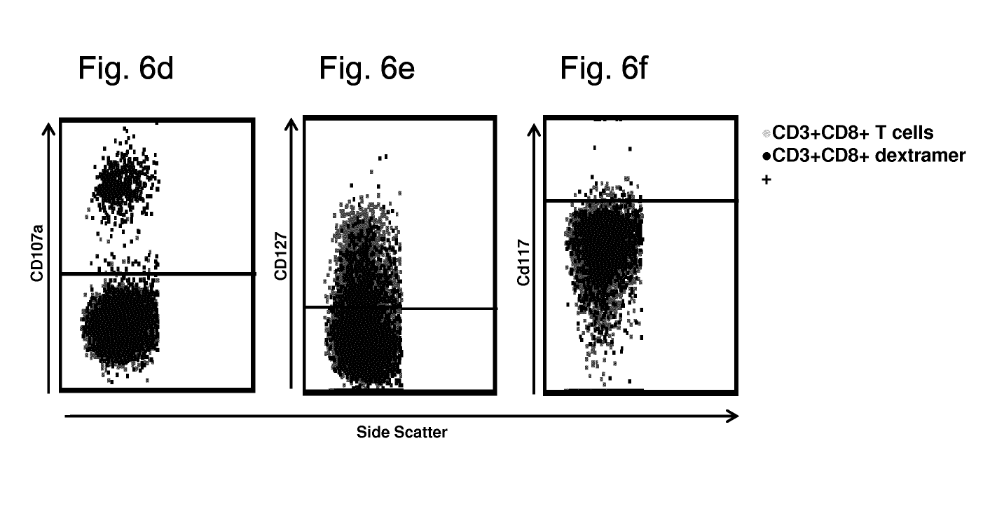

PBMCs were obtained from a patient with glioblastoma. and were expanded with the cytokine cocktail of IL-2, IL-15 and IL-21 according to Example 1 but in combination with CMVpp65 peptide. This experiment shows that the CMVpp65 peptide stimulation in combination with the cytokine cocktail of IL-2, IL-15 and IL-21 leads to a strong expansion of high affinity antigen specific T-cells (tetramer analysis). Results are shown in FIG. 7A to 7D. APC-CMV bzw. PE-CMV und FITC-CMV stand for i) identical MHC class I-HLA-0201 molecules, ii) identical peptides. But the MHC molecule is mutated. The only difference is the mutation in the MHC molecule—which detects T-cells with different affinity. The CMV tetramers are differently mutated. APC-CMV (medium affinity, mutation in the position 245; between PE-CMV (high affinity) und FITC-CMV (wild type, comparably low affinity). The data show that mutant tetramers that only allow high affinity T-cell receptors for binding detect high affinity T-cells (as well as T-cells with low and intermediate affinity) High affinity T-cells are believed to mediate stronger immune effector functions and to be superior in removing tumor cells and/or pathogens.

### Example 9

### Analysis of the Frequency of Tumor Reactive T-Cells after Expansion of Lymphocytes from Peripheral Blood with the Cytokine Cocktail and NY-ESO-1

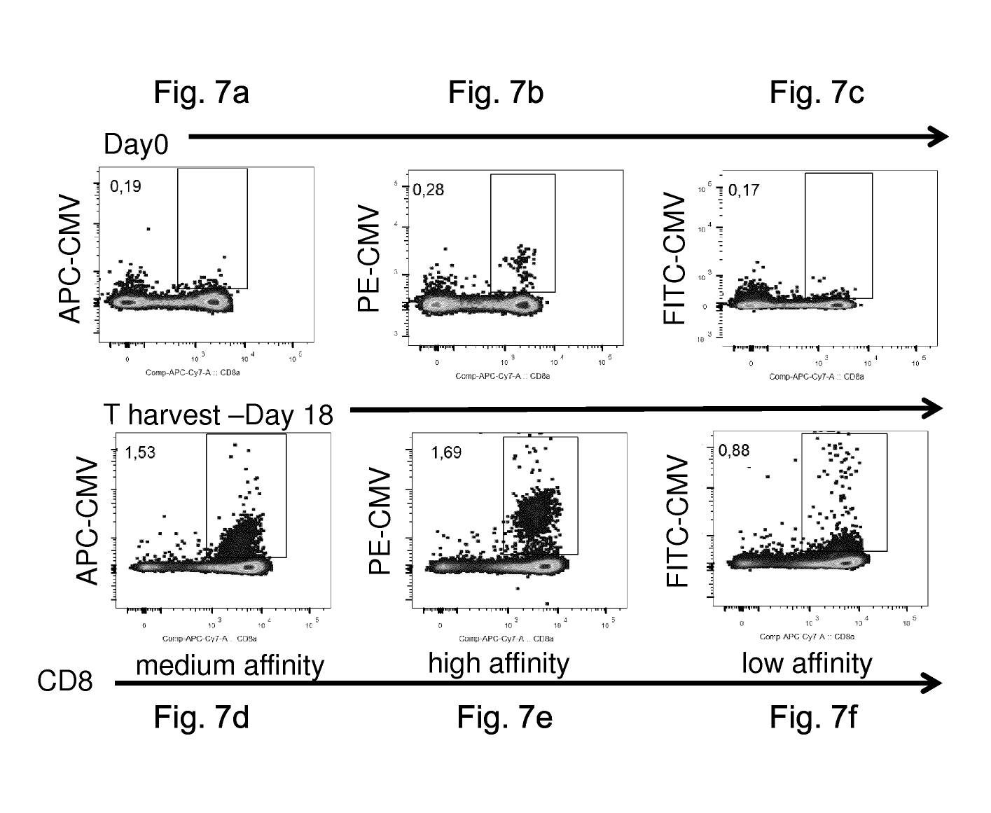

PBMCs were obtained from a patient with pancreatic cancer. The experiment was carried out as described in Example 1. FIG. 8 shows a flow cytometry analysis of samples of the expansion on day 0 and day 18. While on day 1 the IFN-γ production upon NY-ESO-1 stimulation is only 1.85, this concentration increases drastically to 9.25 on day 18 (see FIG. 8B and 8D). Thus the number of NY-ESO-1 specific T-cells is drastically increased during expansion.

### Example 10

### Expansion of Survivin Reactive T-Cells from Peripheral Blood from Patients with Cancer

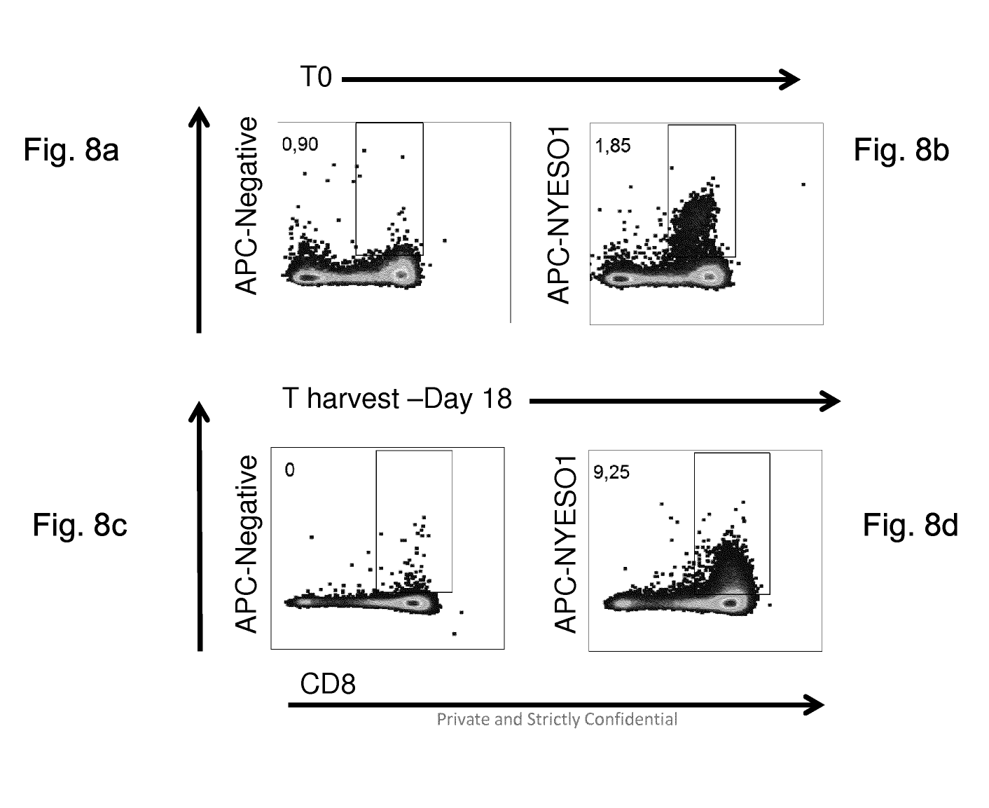

PBMCs were obtained from a patient with glioblastoma and were expanded with the cytokine cocktail of IL-2, IL-15 and IL-21 according to Example 1 but together with the known TAA survivin. Again cells were analyzed on day 1 and after 18 days of culture using flow cytometry. Before analysis the expanded cells were stimulated with survivin. Cells were grouped into CD4+, CD8+ and double negative T-cells. In these groups the concentration of IL-2, IFN-γ and TNF-α was determined. FIG. 9A shows the results for CD4+ cells, FIG. 9B the results for the double negative and FIG. 9C the results for the CD8+ cells. The results can be summarized as follows: there are no detectable T-cell responses, defined by cytokine production at time point 0 (T0). In contrast, after 18 days of expansion strong cytokine production is found in response to stimulation by survivin proving the presence of survivin specific T-cells. In this regard it has to be noted that T-cell responses are generally very difficult to induce against survivin.

### Example 11

### Analysis of the Phenotype of Expanded Lymphocytes

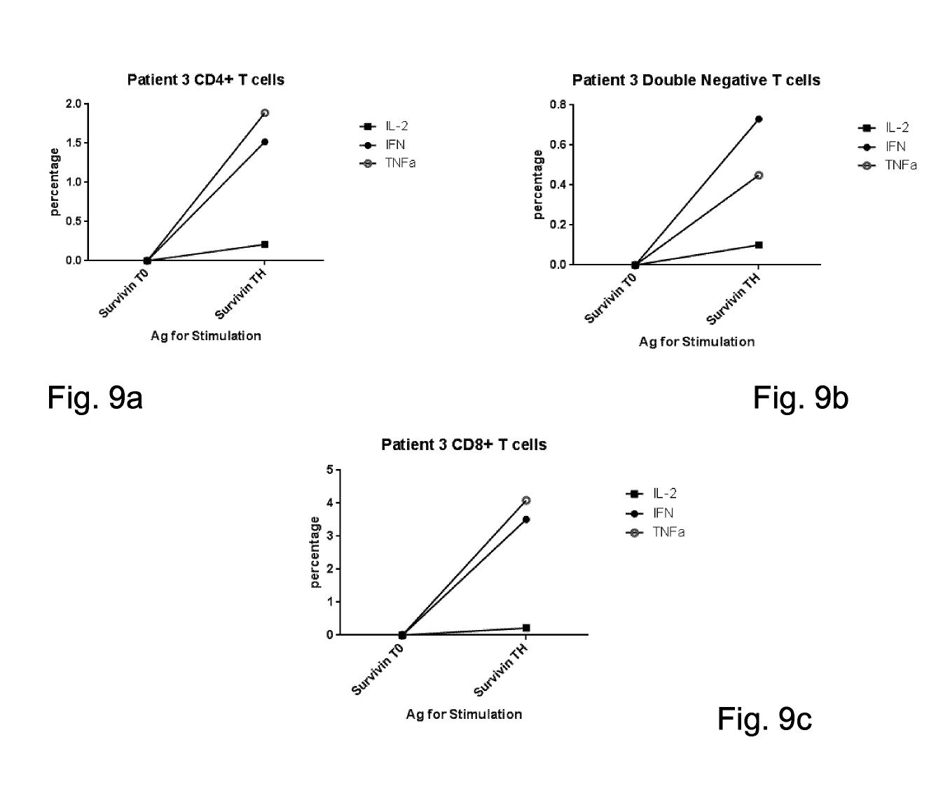

PBMCs were obtained from a patient with glioblastoma. The expansion was carried out as described in Example 1. Samples from day 0 and day 18 were analyzed according to the presence of CD45RA and CCR7 using flow cytometry. The results are shown in FIG. 10A and FIG. 10B. For this experiment, cells were grouped into the following groups. Positive signals for CD45RA and CCR7 upper right section of the graph stands for precursor cells. CCR7+ CD45RA− lower right is central memory cells. CD45RA positive signal with CCR7− upper left area are the effector cells and the negative signal in both regions lower left area are the peripheral memory cells. As shown in comparison of FIG. 10A and FIG. 10B it is obvious that the number of central memory cells due to expansion with the cytokine cocktail strongly increases, i.e. form 3.72 to 21.1. The number of peripheral memory cells slightly increases while the number of effector cells strongly decreases. The number of precursor cells only slightly decreases from 65.4 to 57.4. Accordingly, expansion with the cytokine combination according to the invention enriches central memory T-cells.

### Example 12

### Analysis of the Differentiation of Different T-Cell Subsets upon Expansion with the Cytokine Cocktail of IL-2, IL-15 and IL-21 Together with Different TAA

PBMCs from patients with pancreas cancer were treated according to Example 1 with different antigens: GPI which is the cell surface bound part of mesothelin, survivin and NY-ESO-1. The results are summarized in the following Table 2:

TABLE 2

| | | | | | | | | | Peripheral | |
|---|---|---|---|---|---|---|---|---|---|---|
| | | | Central memory | | Precursor | | Effector T-cells | | memory | |
| | | | CD45RA−CCR7+ | | CD45RA+CCR7+ | | CD45RA+CCR7− | | CD45RA−CCR7− | |
| | T-cell | Antigen | Time- | After | Time- | After | Time- | After | Time- | After |
| | population n | for stimulation | point 0 | stimulation | point 0 | stimulation | point 0 | stimulation | point 0 | stimulation |
| Patient X | CD4+ | GPI | 12.80 | 24.30 | 44.50 | 24.00 | 7.93 | 2.85 | 34.80 | 48.70 |
| | | NY-ESO-1 | | 29.10 | | 12.20 | | 3.71 | | 54.90 |
| | | Survivin | | 15.50 | | 4.24 | | 1.89 | | 78.40 |
| | CD8+ | GPI | 0.61 | 4.80 | 26.70 | 50.90 | 57.60 | 25.50 | 15.10 | 18.80 |
| | | NY-ESO-1 | | 10.90 | | 11.30 | | 11.50 | | 66.30 |
| | | Survivin | | 7.20 | | 8.98 | | 14.50 | | 69.30 |
| | CD4− CD8− | GPI | 1.81 | 4.31 | 29.20 | 19.90 | 58.80 | 40.40 | 10.20 | 35.40 |
| | | NY-ESO-1 | | 6.90 | | 7.44 | | 66.80 | | 18.90 |
| | | Survivin | | 5.49 | | 7.10 | | 57.60 | | 29.80 |

In the first column the T-cell subset to be analyzed is shown: CD4+, CD8+, CD4− and CD8−. In the second column the antigen stimulus is defined. In the following columns the percentage of central memory, precursor, differentiated effector cells and peripheral memory cells is shown at day 0 and day 18 of the different experiments. Accordingly, some antigens such as mesothelin particularly drive the expansion of precursor cells and defined by CD45RA+ CCR7+ and central memory cells defined by CD45RA− CCR7+.

### Example 13

### Enrichment of Precursor T-Cells and C-Kit Expression

PBMCs from patient with glioblastoma were expanded according to the protocol of Example 1 with peptides of the antigens (EGRvIII, NY-ESO-1 or survivin). The cells were stimulated with the same peptides analyzed by flow cytometry and the results are summarized in Table 3:

TABLE 3

| | Patient 1 | | | | Patient 2 | | | |
|---|---|---|---|---|---|---|---|---|
| | EGFRV | NY-ESO-1 | Survivin | TIMEPOINT 0 | EGFRVIII | NY-ESO-1 | Survivin | TIMEPOINT 0 |
| Lymphocytes | 71.2 | 60.3 | 72.7 | 83.2 | 74 | 48.8 | 51.9 | 68.2 |
| CD3+ | 76 | 54.6 | 75.9 | 46.4 | 65.4 | 60.8 | 60.9 | 48.9 |
| CD4+ | 11 | 27.4 | 11.1 | 42.5 | 35 | 63.4 | 61.2 | 47.8 |
| c-kit+ | 22.4 | 55.2 | 21.9 | 0.6 | 33.5 | 75.8 | 71.3 | 2.17 |
| CD107a+ | 5.62 | 17.5 | 5.32 | 0.96 | 2.68 | 14.8 | 13 | 1.47 |
| Q1: CCR7−, CD45RA+ | 5.58 | 10.5 | 5.8 | 23 | 81 | 56.5 | 53.3 | 52.8 |
| Q2: CCR7+, | 59.1 | 13 | 56.5 | 44.2 | 13 | 35.9 | 40.1 | 32.8 |
| CD45RA+ | | | | | | | | |
| Q3: CCR7+, CD45RA− | 15.9 | 27.6 | 16.8 | 11.3 | 1.01 | 3.18 | 3.22 | 7.4 |
| Q4: CCR7−, CD45RA− | 19.4 | 48.9 | 20.9 | 21.5 | 5 | 4.48 | 3.44 | 7.02 |
| CD8+ | 33.5 | 28.5 | 33.7 | 20.9 | 27.6 | 11.3 | 8.82 | 13.3 |
| c-kit+ | 40.6 | 33.4 | 40.7 | 0.66 | 23 | 90.8 | 91.9 | 1.08 |
| CD6aa | 29.9 | 48.3 | 33.7 | 49 | 42.1 | 49 | 50.2 | 40.4 |
| CD6ab | 66.5 | 48.4 | 62.3 | 47 | 53.9 | 46.6 | 45.7 | 55.9 |
| CD107a+ | 1.32 | 5.12 | 1.18 | 1.45 | 0.77 | 15 | 15.1 | 4 |
| Q1: CCR7−, CD45RA+ | 15.8 | 42.2 | 14.1 | 51.6 | 92.4 | 75.8 | 69.7 | 68.9 |
| Q2: CCR7+, | 9.11 | 9.98 | 7.66 | 25.3 | 3.8 | 15.1 | 24.4 | 24.6 |
| CD45RA+ | | | | | | | | |
| Q3: CCR7+, CD45RA− | 5.02 | 3.51 | 5.8 | 2.47 | 0.11 | 0.84 | 1.19 | 2.34 |
| Q4: CCR7−, CD45RA− | 70.1 | 44.3 | 72.5 | 20.6 | 3.73 | 8.26 | 4.76 | 4.13 |
| DN | 54.7 | 38 | 54.6 | 36.2 | 36.3 | 15.2 | 22.4 | 38.6 |
| c-kit+ | 11.1 | 9.35 | 11.4 | 2.1 | 5.88 | 24.9 | 46.9 | 1.72 |
| CD107a+ | 5.36 | 14.2 | 4.39 | 2.5 | 3.12 | 19 | 13.7 | 3.93 |
| Q1: CCR7−, CD45RA+ | 22.2 | 39.6 | 20.6 | 46.4 | 88.8 | 54.8 | 67 | 54.5 |
| Q2: CCR7+, | 3.9 | 12.4 | 3.34 | 32.1 | 4.43 | 21.8 | 19.8 | 37.4 |
| CD45RA+ | | | | | | | | |
| Q3: CCR7+, CD45RA− | 4.02 | 7.93 | 4.05 | 5.41 | 0.76 | 8.17 | 3.13 | 4.11 |
| Q4: CCR7−, CD45RA− | 69.9 | 40.1 | 72 | 16.2 | 5.99 | 15.2 | 10.1 | 4.02 |

| | Patient 3 | | | |
|---|---|---|---|---|
| | EGFRV | NY-ESO-1 | Survivin | TIMEPOINT 0 |
| Lymphocytes | 71.4 | 67.1 | 62.8 | 85.1 |
| CD3+ | 57.7 | 63.8 | 66.5 | 53.9 |
| CD4+ | 9.05 | 24.9 | 23.6 | 35.8 |
| c-kit+ | 6.44 | 27.6 | 30.3 | 0.29 |
| CD107a+ | 2.56 | 8.5 | 12.2 | 0.69 |
| Q1: CCR7−, CD45RA+ | 1.49 | 7.67 | 6.67 | 4.46 |
| Q2: CCR7+, | 59.6 | 36.7 | 43.1 | 41.3 |
| CD45RA+ | | | | |
| Q3: CCR7+, CD45RA− | 20.7 | 20.5 | 21.1 | 14.7 |
| Q4: CCR7−, CD45RA− | 18.2 | 35.1 | 29.2 | 39.5 |
| CD8+ | 24.3 | 30 | 34.8 | 36 |
| c-kit+ | 11.4 | 49.3 | 32.7 | 0.078 |
| CD6aa | 23.9 | 16 | 5.61 | 32.7 |
| CD6ab | 73.8 | 82.2 | 93.5 | 64.4 |
| CD107a+ | 2.12 | 4.55 | 5.6 | 2.44 |
| Q1: CCR7−, CD45RA+ | 14.5 | 15 | 30.2 | 37.1 |
| Q2: CCR7+, | 23.9 | 41.1 | 50.1 | 37.3 |
| CD45RA+ | | | | |
| Q3: CCR7+, CD45RA− | 2.34 | 6.41 | 2.53 | 1.96 |
| Q4: CCR7−, CD45RA− | 59.2 | 37.5 | 17.1 | 23.6 |
| DN | 66.4 | 37.2 | 31.5 | 27.9 |
| c-kit+ | 5.05 | 15.4 | 5.05 | 0.23 |
| CD107a+ | 3.37 | 15.5 | 17.6 | 3.76 |
| Q1: CCR7−, CD45RA+ | 30.4 | 20.6 | 13.3 | 51.3 |
| Q2: CCR7+, | 1.77 | 19.9 | 36.7 | 10.6 |
| CD45RA+ | | | | |
| Q3: CCR7+, CD45RA− | 1.52 | 10.5 | 15.3 | 3.13 |
| Q4: CCR7−, CD45RA− | 66.3 | 49 | 34.7 | 35 |

The numbers are percentages of the T-cell populations in the parental CD4+, CD8+, or double negative phenotype for each of the respective antigens at time point 18 days with comparison to time point 0 (start of the expansion). An enrichment of precursor T-cell subsets, defined by CD45RA+ CCR7+ associated with antigen and strong expression of c-kit (CD117) is detected. C-kit is a marker for T-cells with stem-cell like features. CD117+ T-cells have been associated to provide long-term memory population. Strong induction of the cytotoxicity/degranulation marker CD107a in stimulated T-cells is also observed.

### Example 14

### The Expansion Protocol with the Cytokine Cocktail and TAA Peptide Leads to 4-1 BB and TIM-3 positive T-Cells

PBMCs were derived from patients with pancreatic cancer and expanded according to Example 1. Again TAA peptides were derived from GPI, NY-ESO-1 and survivin. Using flow cytometry the presence of different markers was tested. The markers are: 4-1BB, CD25, CD127, CTLA-4, LAG3, PD1 and TIM3. The results are summarized in Table 4:

TABLE 4

| | | | 4-1BB | | CD25+ | | CD127+ | | CTLA-4+ | | LAG3+ | | PD1+ | | TIM3+ | |
|---|---|---|---|---|---|---|---|---|---|---|---|---|---|---|---|---|
| | | | T 0 | Ag | T 0 | Ag | T 0 | Ag | T 0 | Ag | T 0 | Ag | T 0 | Ag | T 0 | Ag |
| Patient | CD4+ | GPI | 0.03 | 0.33 | 6.47 | 37.00 | 66.70 | 36.80 | 0.01 | 0.01 | 0.16 | 0.18 | 8.22 | 26.10 | 0.00 | 0.02 |
| A | | NY-ESO-1 | | 5.96 | | 25.80 | | 45.30 | | 0.00 | | 0.28 | | 25.70 | | 0.17 |
| | | Survivin | | 2.17 | | 32.10 | | 37.50 | | 0.01 | | 0.24 | | 25.80 | | 0.61 |
| | CD6+ | GPI | 0.04 | 0.03 | 1.46 | 23.90 | 23.40 | 1.77 | 0.10 | 0.06 | 0.85 | 0.29 | 7.43 | 39.20 | 0.00 | 0.01 |
| | | NY-ESO-1 | | 0.15 | | 39.50 | | 2.14 | | 0.10 | | 0.41 | | 44.60 | | 0.03 |
| | | Survivin | | 0.11 | | 37.10 | | 2.47 | | 0.15 | | 0.56 | | 39.70 | | 0.03 |
| | CD4− CD6− | GPI | 0.04 | 0.14 | 0.89 | 28.10 | 12.90 | 1.05 | 0.07 | 0.02 | 0.32 | 0.17 | 12.00 | 23.70 | 0.00 | 0.06 |
| | | NY-ESO-1 | | 0.89 | | 40.70 | | 2.06 | | 0.04 | | 0.17 | | 11.60 | | 0.29 |
| | | Survivin | | 0.41 | | 50.10 | | 1.84 | | 0.03 | | 0.23 | | 12.50 | | 0.43 |
| Patient | CD4+ | GPI | 0.06 | 1.47 | 4.26 | 78.30 | 54.70 | 27.30 | 0.03 | 0.20 | 0.45 | 0.35 | 17.10 | 62.70 | 0.00 | 0.43 |
| B | | NY-ESO-1 | | 1.80 | | 56.30 | | 19.20 | | 0.06 | | 0.42 | | 28.90 | | 0.25 |
| | | Survivin | | 3.78 | | 75.10 | | 27.50 | | 0.21 | | 0.43 | | 61.90 | | 0.51 |
| | CD6+ | GPI | 0.05 | 1.51 | 2.15 | 55.30 | 18.00 | 7.74 | 0.10 | 0.21 | 0.82 | 3.97 | 11.80 | 47.60 | 0.00 | 0.34 |
| | | NY-ESO-1 | | 1.52 | | 40.40 | | 6.07 | | 0.13 | | 1.16 | | 30.80 | | 0.16 |
| | | Survivin | | 2.71 | | 56.40 | | 7.45 | | 0.33 | | 1.78 | | 44.10 | | 0.39 |
| | CD4− CD6− | GPI | 0.00 | 3.19 | 0.87 | 41.60 | 7.80 | 4.94 | 0.29 | 0.52 | 0.87 | 0.75 | 10.70 | 31.60 | 1.07 | 3.12 |
| | | NY-ESO-1 | | 3.40 | | 25.90 | | 6.49 | | 0.72 | | 0.48 | | 23.40 | | 2.01 |
| | | Survivin | | 2.50 | | 32.10 | | 7.01 | | 2.50 | | 0.44 | | 29.50 | | 0.00 |

In the Table 4 T0 stands for the concentration of the individual subsets defined in column 2, prior to expansion (T0). Ag stands for 18 days of antigenic stimulation and cytokine-driven expansion. The 4-1BB values and TIM3 values are indicative for antigen experienced T-cells. It is found that in particular 4-1BB+ T-cells are strongly increased in some of the experiments and cellular subsets. For example in the CD4+ subset stimulation with NY-ESO-1 leads to an 200-fold increase in 4-1BB+ T-cells (from 0.03 to 5.96).

### Example 15

### Detailed Analysis of Cellular Markers in Lymphocytes Expanded from Peripheral Blood of Patients with Glioblastoma

The experiment was carried out according to the protocol of Example 1 with EGVRVIII, NY-ESO-1 or survivin, respectively. The results are summarized in Table 5.

TABLE 5

| | | | Patient 1 | | Patient 2 | | Patient 3 | | Patient 4 | | Patient 5 | |
|---|---|---|---|---|---|---|---|---|---|---|---|---|
| | | Stim Ag | T0 | Ag | T0 | Ag | T0 | Ag | T0 | Ag | T0 | Ag |
| CD3+/ | CD4+ | EGFRVIII | 40.4 | 12.2 | 70 | 39 | 36.2 | 10.9 | 48.8 | 44.3 | 65.6 | 74.4 |
| CD4+ | | NY-ESO-1 | | 30.5 | | 62.5 | | 30.6 | | 63.1 | | 74.8 |
| | | Survivin | | 24.8 | | 70.5 | | 33.5 | | 63.7 | | 72.2 |
| | 4-1BB+ | EGFRVIII | 0.13 | 1.39 | 0.16 | 1.85 | 0.032 | 0.39 | 0.28 | 0.91 | 0.083 | 1.47 |
| | | NY-ESO-1 | | 26.5 | | 50.2 | | 16.9 | | 3.47 | | 2.78 |
| | | Survivin | | 34.2 | | 43.4 | | 14.6 | | 11.9 | | 4.02 |
| | CD25+ | EGFRVIII | 2.76 | 31.8 | 6.43 | 50.6 | 4.01 | 12 | 10.1 | 84.3 | 4.73 | 86.9 |
| | | NY-ESO-1 | | 66 | | 85.7 | | 29.6 | | 60.5 | | 97.5 |
| | | Survivin | | 56.4 | | 88.2 | | 30.6 | | 76 | | 94.2 |
| | CD127+ | EGFRVIII | 77.6 | 77.5 | 69.5 | 34.7 | 88.5 | 84.9 | 35.7 | 16.4 | 42 | 8.08 |
| | | NY-ESO-1 | | 60.4 | | 49 | | 64.3 | | 34.3 | | 2.08 |
| | | Survivin | | 55.8 | | 54 | | 71.5 | | 35.8 | | 6.77 |
| | CTLA- | EGFRVIII | 0.27 | 0.88 | 1.04 | 1.79 | 0.19 | 0.44 | 1.64 | 1.19 | 1.28 | 3.82 |
| | 4+ | NY-ESO-1 | | 6.87 | | 7.59 | | 0.93 | | 3.03 | | 3.66 |
| | | Survivin | | 4.91 | | 7.43 | | 1.46 | | 7.28 | | 7.04 |
| | LAG3+ | EGFRVIII | 10.7 | 7.71 | 12.5 | 10.1 | 4.05 | 4.68 | 3.66 | 2.73 | 2.21 | 3.06 |
| | | NY-ESO-1 | | 20.8 | | 26.3 | | 10.8 | | 4.65 | | 2.58 |
| | | Survivin | | 20.5 | | 17 | | 13.9 | | 5.98 | | 3.79 |
| | PD-1 | EGFRVIII | 7.1 | 21.9 | 37.8 | 34.2 | 14.8 | 9.13 | 3.99 | 29.3 | 4.64 | 84.5 |
| | | NY-ESO-1 | | 55.8 | | 50.2 | | 15.1 | | 39.4 | | 87.2 |
| | | Survivin | | 47.1 | | 55.4 | | 17.2 | | 55.8 | | 90.5 |
| | TIM3+ | EGFRVIII | 5E−03 | 0.066 | 5E−03 | 0.056 | 0 | 0.016 | 0.13 | 0.35 | 0.044 | 0.75 |
| | | NY-ESO-1 | | 0.34 | | 0.36 | | 0.063 | | 0.52 | | 0.066 |
| | | Survivin | | 0.28 | | 0.27 | | 0.5 | | 1.71 | | 0.21 |
| CD3+/ | CD8+ | EGFRVIII | 18.2 | 34.7 | 19.6 | 30.8 | 39.6 | 25.3 | 20.1 | 34.9 | 9.55 | 16 |
| CD8+ | | NY-ESO-1 | | 33.9 | | 9.63 | | 33.2 | | 25.5 | | 13.2 |
| | | Survivin | | 36.2 | | 11.1 | | 45 | | 22.2 | | 15.1 |
| | 4-1BB+ | EGFRVIII | 1.64 | 0.78 | 1.16 | 0.49 | 0.66 | 0.47 | 0.067 | 0.96 | 0.17 | 1.93 |
| | | NY-ESO-1 | | 13.3 | | 24.8 | | 12.5 | | 7.57 | | 10.6 |
| | | Survivin | | 12.4 | | 25.2 | | 11 | | 16.2 | | 11.6 |
| | CD25+ | EGFRVIII | 5.58 | 59.3 | 5.98 | 44.1 | 1.59 | 25.8 | 3.47 | 90.1 | 1.4 | 86.9 |
| | | NY-ESO-1 | | 43 | | 97.6 | | 65.2 | | 50.9 | | 96 |
| | | Survivin | | 45.4 | | 94.6 | | 44.4 | | 65 | | 94.4 |
| | CD127+ | EGFRVIII | 68.8 | 32.6 | 62.7 | 30 | 67.4 | 43.6 | 42.5 | 15.6 | 52.6 | 21.6 |
| | | NY-ESO-1 | | 47.6 | | 57.1 | | 49.4 | | 37 | | 12.1 |
| | | Survivin | | 42.9 | | 53.1 | | 57.1 | | 37.1 | | 19.6 |
| | CTLA- | EGFRVIII | 1.32 | 1.17 | 2.16 | 1.48 | 1.37 | 1.62 | 12 | 8.46 | 14.3 | 24.2 |
| | 4+ | NY-ESO-1 | | 3.58 | | 8.89 | | 2.08 | | 19.4 | | 15.6 |
| | | Survivin | | 2.91 | | 7.09 | | 2.91 | | 18.3 | | 28 |
| | LAG3+ | EGFRVIII | 79 | 92.3 | 88.3 | 94.5 | 91.9 | 96.1 | 15.9 | 12.6 | 18.9 | 26.2 |
| | | NY-ESO-1 | | 94.5 | | 93.1 | | 98.3 | | 25.6 | | 14.3 |
| | | Survivin | | 95.5 | | 94.6 | | 98.1 | | 26.5 | | 24.6 |
| | PD-1 | EGFRVIII | 7.53 | 18.2 | 26.3 | 39.5 | 4.63 | 14.4 | 6.04 | 21.4 | 5.34 | 56.5 |
| | | NY-ESO-1 | | 28.3 | | 44 | | 18.2 | | 26.4 | | 80.4 |
| | | Survivin | | 29.4 | | 41.9 | | 12.8 | | 37 | | 82.7 |
| | TIM3+ | EGFRVIII | 0 | 0.21 | 0.016 | 0.11 | 7E−03 | 0.15 | 0.12 | 0.69 | 0.13 | 2.93 |
| | | NY-ESO-1 | | 0.061 | | 0.11 | | 0.27 | | 1.72 | | 1.2 |
| | | Survivin | | 0.19 | | 0.2 | | 1.75 | | 4.13 | | 3.96 |

Again the lymphocytes are separated according to the presence of CD4 or CD8+. The numbers in the table refer to percentages of cells positive for the individual markers listed in the second column in the table at T0 or after 18 days of expansion (Ag). Again for most patients a strong induction of 4-1BB+ T-cells is found.

### Example 16

### Analysis of Lymphocytes Expanded from a Patient with Pancreatic Cancer and from a Patient with Glioblastoma

PBMCs were obtained from a patient with pancreatic cancer and from a patient with glioblastoma. The lymphocytes were expanded with the protocol according to Example 1. The lymphocyte culture is analyzed by flow cytometry against a variety of markers. The results are represented in Table 6:

TABLE 6

| | | Panc | | GBM | |
|---|---|---|---|---|---|
| | | T0 | TH | T0 | TH |
| CD3 | | 64 | 69, 9 | 70 | 88, 6 |
| CD4 | | 59, 1 | 31, 2 | 64 | 11 |
| | CCR6 Neg | 88, 9 | 97, 4 | 95, 9 | 86, 6 |
| | TH1 | 9, 69 | 97, 1 | 4, 82 | 38, 8 |
| | TH2 | 0, 23 | 0 | 0, 64 | 0, 2 |
| | CCR6 Pos | 10, 1 | 2, 03 | 3, 25 | 12 |
| | TH1* | 22, 2 | 92, 1 | 9, 64 | 68, 1 |
| | TH17 | 1, 05 | 0 | 1, 02 | 0, 52 |
| | CCR7− | 14, 5 | 13, 8 | 2, 69 | 2, 06 |
| | CD45RA+ | | | | |
| | CCR7+ | 12, 1 | 0, 25 | 49 | 61, 9 |
| | CD45RA+ | | | | |
| | CCR7+ | 10, 9 | 0, 2 | 21, 2 | 23, 3 |
| | CD45RA− | | | | |
| | CCR7− | 62, 5 | 85, 7 | 27, 1 | 12, 7 |
| | CD45RA− | | | | |
| | C-KIT | 0, 52 | 0, 051 | 0, 77 | 3, 12 |
| | CD107a | 0, 4 | 0, 14 | 0, 8 | 4, 39 |
| CD8 | | 33, 5 | 64, 5 | 30, 3 | 71, 2 |
| | CXCR3+ | 6, 67 | 96, 8 | 17 | 85, 5 |
| | CCR7− | 61, 1 | 30, 8 | 20, 1 | 7, 39 |
| | CD45RA+ | | | | |
| | CCR7+ | 9, 91 | 0, 87 | 66, 8 | 66, 2 |
| | CD45RA+ | | | | |
| | CCR7+ | 2, 29 | 0, 082 | 2, 52 | 10, 9 |
| | CD45RA− | | | | |
| | CCR7− | 26, 6 | 68, 2 | 10, 5 | 15, 5 |
| | CD45RA− | | | | |
| | C-KIT | 2, 17 | 0, 22 | 2, 85 | 4, 34 |
| | CD107a | 3, 45 | 0, 34 | 4, 23 | 5, 66 |
| DN | | 6, 15 | 3, 47 | 4, 56 | 13, 7 |
| | CXCR3+ | 8, 67 | 87, 3 | 24 | 58, 3 |
| | CCR7− | 73, 1 | 37, 2 | 41, 7 | 15, 7 |
| | CD45RA+ | | | | |
| | CCR7+ | 5, 63 | 0, 093 | 20, 2 | 7, 12 |
| | CD45RA+ | | | | |
| | CCR7+ | 3, 44 | 0, 46 | 5, 95 | 5, 6 |
| | CD45RA− | | | | |
| | CCR7− | 17, 8 | 62, 3 | 32, 1 | 71, 6 |
| | CD45RA− | | | | |
| | C-KIT | 4, 33 | 0, 51 | 1, 67 | 2, 24 |
| | CD107a | 8, 91 | 1, 2 | 1, 67 | 2, 9 |

In the first column it is indicated what subgroup of lymphocytes was filtered. In the second column the respective tested markers are listed. If no marker is listed this line represents the percentage of total number of cells with the filter marker CD3+ based on the total number of cells and the CD4+, CD8+ or double negative. Again T0 stands for the culture prior to expansion and TH for the time of harvest at day 18. The numbers represent the percentage of cells carrying the selected markers in column 2 or column 1 respectively. It has to be noted that majority of CD4+ cells after expansion is TH1 positive. In pancreatic cancer the numbers increase from 22.2 to 92.1% of the CD4 cells. In glioblastoma the results are only little less pronounced from 9.64 to 68.1% of the CD4 cells. Likewise, moreover there is a strong expression of CXCR3+ in CD8+ T-cells after expansion 96.8% in pancreas and 85.5% in glioblastoma. Accordingly almost all of the CD8+ cells are CXCR3+.

Moreover it is noted a shift away from terminally differentiated T-cells into precursor or central memory T-cell subsets.

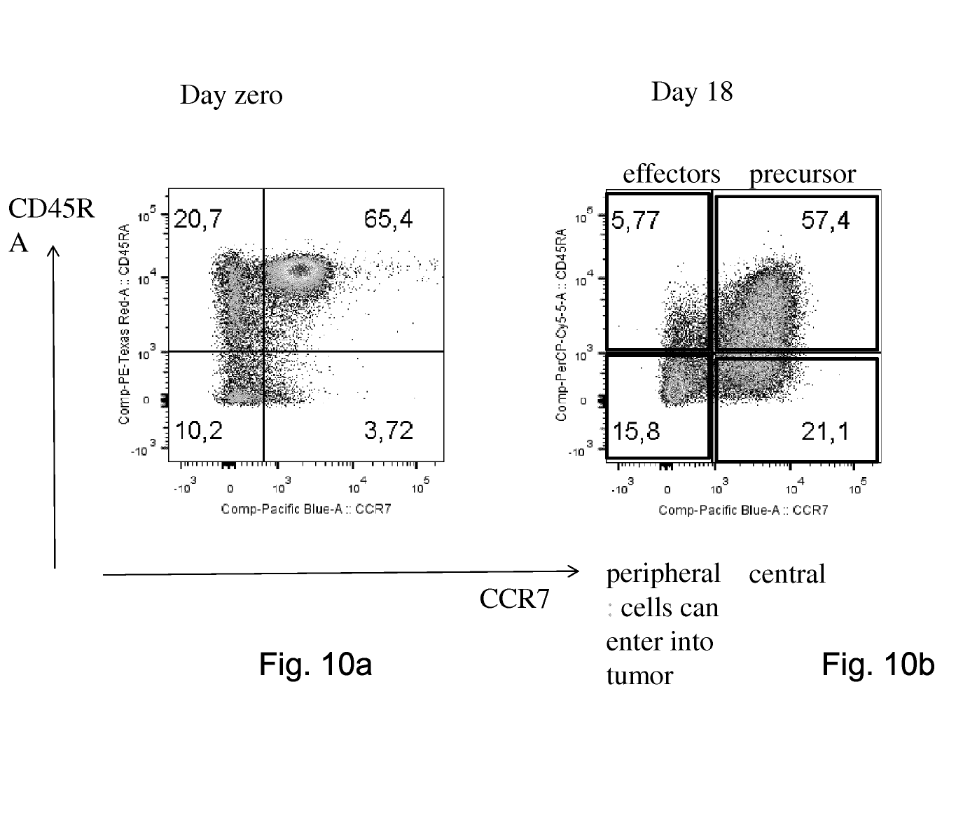

FIG. 11 shows flow cytometry graphs of the experiment for the determination of TH17, TH1, TH1* and TH2 in the sample from the pancreas cancer patient First the cells are gated on CCR6 and then on CCR3 and CCR4 respectively.

### Example 17

### Expansion of Memory T-Cells Specific for Certain Antigens in the Absence of Stimulation with these Antigens

PBMCs from patients with pancreas cancer were expanded according to Example 1 with the tumor associated antigen NY-ESO-1. The resulting cells were stimulated for four hours with the following tumor associated antigens: CMV, EBNA-3a, INO80E, mesothelin, NY-ESO-1, survivin and UCHL3. Using flow cytometry the percentage of cytokine producing T-cells was determined. The numbers are represented in Table 7 for different T-cell subsets: CD8+, CD4+ and double negative. The following cytokines were tested: IFN-γ, IL-2 and TNF. Interestingly, not only stimulation with NY-ESO-1 led to cytokine producing T-cells and therefore tumor reactive T-cells but also stimulation with other antigens that were not used in the expansion process. Also stimulation with antigens of viral targets (CMV and EBNA-3a) led to T-cells producing cytokines showing the presence of T-cells specific for these targets.

TABLE 7

| AFTER | | AFTER MEDIUM SUBSTRACTION | | | | | | | |
|---|---|---|---|---|---|---|---|---|---|
| EXPANSION | | PMA | CMV | EBNA-3a | INO80E | Mesothelin | NY-ESO-1 | Survivin | UCHL3 |
| CD8+ | IFN+ | 20.03 | 0.97 | 1.56 | 0.00 | 0.10 | 0.14 | 0.07 | 0.13 |
| | IL-2+ | 3.84 | 0.06 | 0.02 | 0.00 | 0.04 | 0.03 | 0.04 | 0.06 |
| | TNF+ | 21.61 | 1.49 | 1.46 | 0.07 | 0.41 | 0.27 | 0.25 | 1.01 |
| CD4+ | IFN+ | 10.08 | 0.00 | 0.00 | 0.00 | 0.00 | 0.00 | 0.00 | 0.00 |
| | IL-2+ | 14.39 | 0.22 | 0.06 | 0.09 | 0.16 | 0.15 | 0.10 | 0.33 |
| | TNF+ | 10.29 | 0.00 | 0.00 | 0.00 | 0.02 | 0.00 | 0.00 | 0.65 |
| DN | IFN+ | 5.53 | 0.05 | 0.14 | 0.00 | 0.00 | 0.00 | 0.00 | 0.00 |
| | IL-2+ | 5.89 | 0.20 | 0.14 | 0.02 | 0.09 | 0.22 | 0.19 | 0.23 |
| | TNF+ | 4.82 | 1.15 | 0.36 | 0.34 | 1.22 | 0.59 | 0.56 | 2.18 |

### Example 18

### Cytotoxicity after HPV L1 Specific Expansion

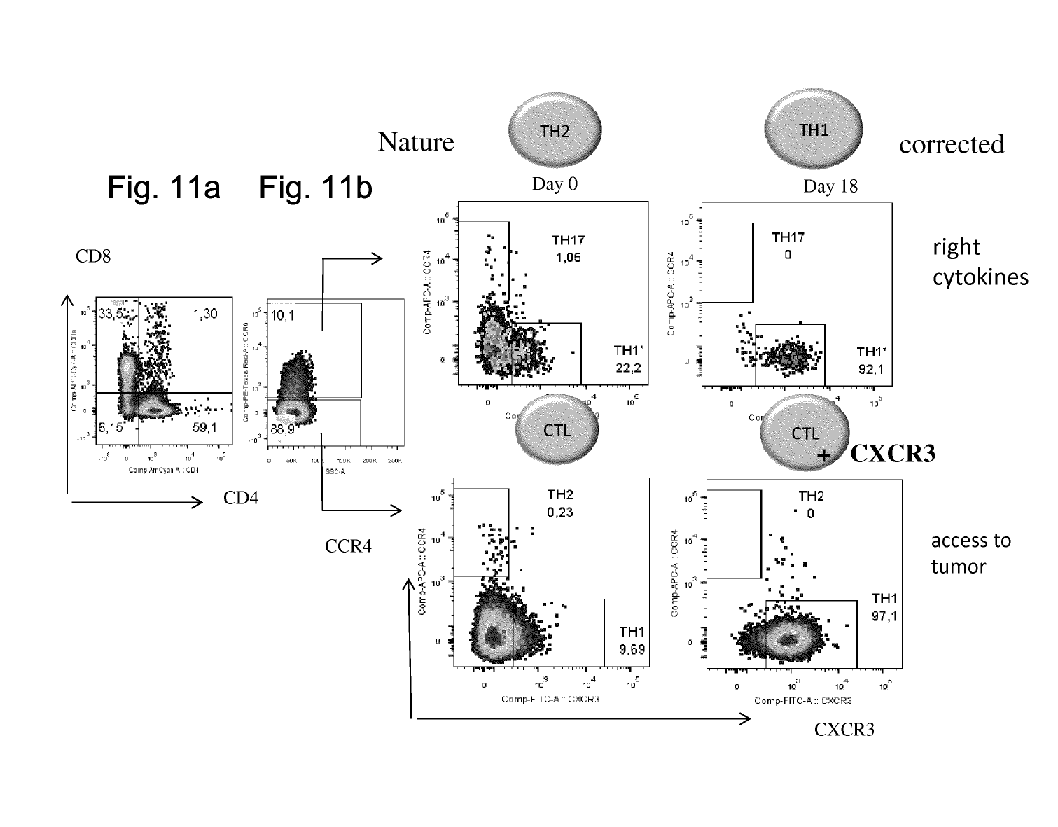

In this example peripheral blood was obtained from a patient who suffered from severe HPV disease (HPV33 and HPV56). The stimulation was performed with the HPV L1 peptide and the cytokine cocktail comprising IL-2, IL-15 and IL-21. After 18 days of expansion the cells were stimulated with L1 peptide and measured by flow cytometry. The results of this are shown in FIG. 12. FIG. 12D to 12F show the gating for lymphocytes CD3+ and CD8+ respectively. In FIGS. 12A, 12B and 12C the rectangle marks the area showing cells that express cytokine CD107a, a marker for degranulation/cytotoxicity that is antigen-specific. FIG. 12A the results obtained with the L1 peptide, FIG. 12B a positive control and FIG. 12C just medium as negative control. Accordingly there is an increase from 0.77 to 2.23 in the percentage of cells producing CD107a.

### Example 19

### T-Cells Expanded with the Cytokine Cocktail and Tumor Antigens Recognize Autologous Tumor Cells

PBMCs from a patient with glioblastoma were stimulated with NY-ESO-1 peptides and CMVpp65 peptides. Peripheral blood from a patient with glioblastoma was treated according to the protocol of Example 4 wherein in two setups the stimulating peptide was either NY-ESO-1 or CMVpp65. Again after 18 days a cytokine production assay was performed using different antigens for stimulation or the autologous tumor cells. It was found that antigen-specific expansion of T-cells using peptides and the cytokine cocktail leads to expansion of T-cells directed against the stimulating target, yet also against the patient's own autologous tumor cells. This reactivity was not present prior to expansion of T-cells with NY-ESO-1 and cytokines. The data is summarized in Table 8. Numbers represent percentages of T-cells present in the parental T-cell population.

TABLE 8

| | | PMA | | NY-ESO-1 | | Survivin | | CMV | | Tumor | | Medium | |
|---|---|---|---|---|---|---|---|---|---|---|---|---|---|
| GBM | | T0 | TH | T0 | TH | T0 | TH | T0 | TH | T0 | TH | T0 | TH |
| CD4+ | IFN-γ | 16.35 | 45.20 | 0.00 | 0.33 | 0.00 | 0.30 | 0.00 | 0.66 | 0.00 | 0.23 | 0.00 | 0.11 |
| | IL-2 | 50.58 | 64.15 | 0.00 | 0.15 | 0.00 | 0.05 | 0.00 | 0.08 | 0.05 | 0.21 | 0.02 | 0.05 |
| | IL- | 0.69 | 2.09 | 0.02 | 0.08 | 0.01 | 0.11 | 0.00 | 0.01 | 0.04 | 0.21 | 0.03 | 0.09 |
| | 17 | | | | | | | | | | | | |
| | TNF-α | 45.38 | 68.84 | 0.00 | 0.42 | 0.00 | 0.20 | 0.00 | 0.45 | 0.10 | 0.28 | 1.68 | 0.42 |
| CD8+ | IFN-γ | 13.05 | 87.16 | 0.01 | 0.91 | 0.01 | 1.20 | 0.04 | 2.53 | 0.00 | 0.82 | 0.00 | 0.64 |
| | IL-2 | 28.69 | 67.21 | 0.03 | 0.07 | 0.01 | 0.10 | 0.02 | 0.10 | 0.02 | 0.08 | 0.01 | 0.04 |
| | IL- | 0.08 | 0.46 | 0.04 | 0.07 | 0.04 | 0.18 | 0.04 | 0.13 | 0.00 | 0.12 | 0.01 | 0.08 |
| | 17 | | | | | | | | | | | | |
| | TNF-α | 14.28 | 63.99 | 0.00 | 0.36 | 0.00 | 0.45 | 0.00 | 1.13 | 0.00 | 0.15 | 2.02 | 0.41 |
| CD4− | IFN-γ | 8.11 | 63.77 | 0.00 | 2.89 | 0.00 | 3.20 | 0.25 | 7.73 | 0.07 | 2.38 | 1.89 | 0.00 |
| CD8− | IL-2 | 56.02 | 41.42 | 0.03 | 0.32 | 0.00 | 0.39 | 0.25 | 0.10 | 0.30 | 0.40 | 0.13 | 0.14 |
| | IL- | 0.22 | 0.11 | 0.16 | 0.02 | 0.00 | 0.12 | 0.15 | 0.04 | 0.32 | 0.15 | 0.06 | 0.04 |
| | 17 | | | | | | | | | | | | |
| | TNF-α | 28.78 | 48.80 | 0.00 | 1.88 | 0.17 | 2.15 | 0.16 | 5.14 | 0.00 | 1.11 | 1.95 | 1.03 |

### Example 20

### Comparison of Lymphocyte Expansion with Two Different Cytokine Combinations and TAA Stimulation

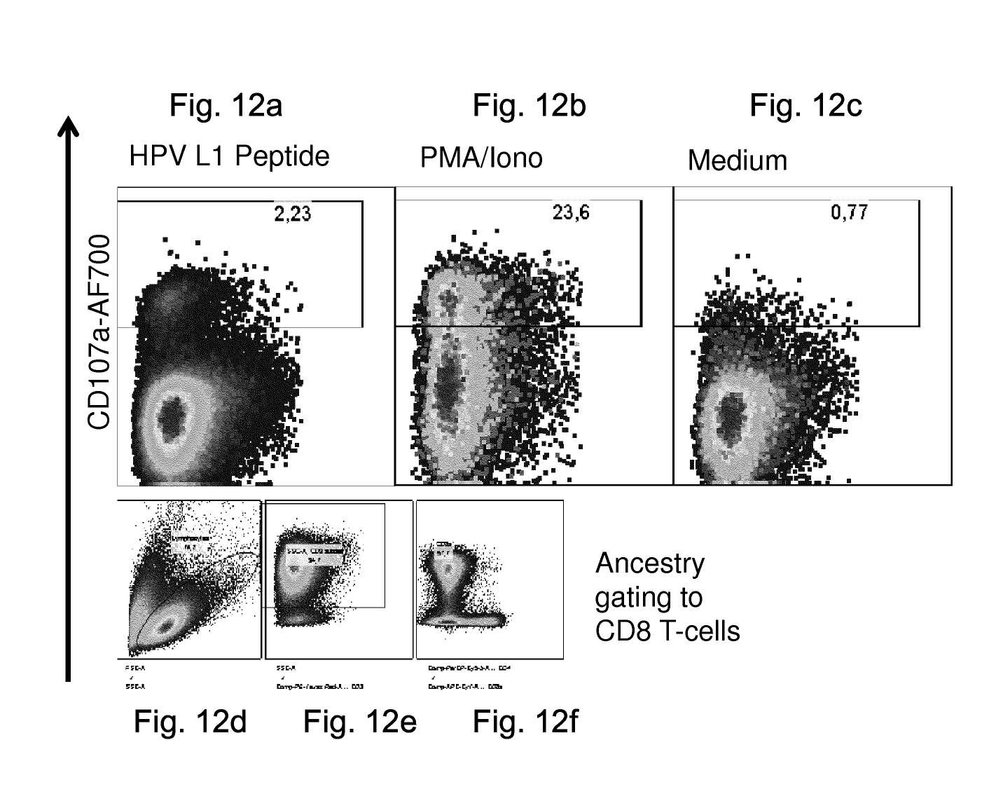

Peripheral blood from patients with pancreatic cancer was obtained and treated according to the protocol of Example 4. In different experimental setups either NY-ESO-1 or survivin was used together with the cytokine cocktail. In addition the same experiments were carried out without the addition of cytokines or with a combination of IL-7 and IL-2. After 18 days of expansion the cells were tested for their IFN-γ production upon stimulation with either NY-ESO-1 or survivin peptide mix. The results are presented in FIG. 13. FIG. 13 shows strongly increased IFN-γ production and thus concentration of antigen-specific lymphocytes after expansion with IL-2, IL15 and IL-21 in comparison to the other cytokine composition or no cytokines.

### Example 21

### Comparison of Lymphocyte Expansion with Two Different Cytokine Combinations and TAA Stimulation

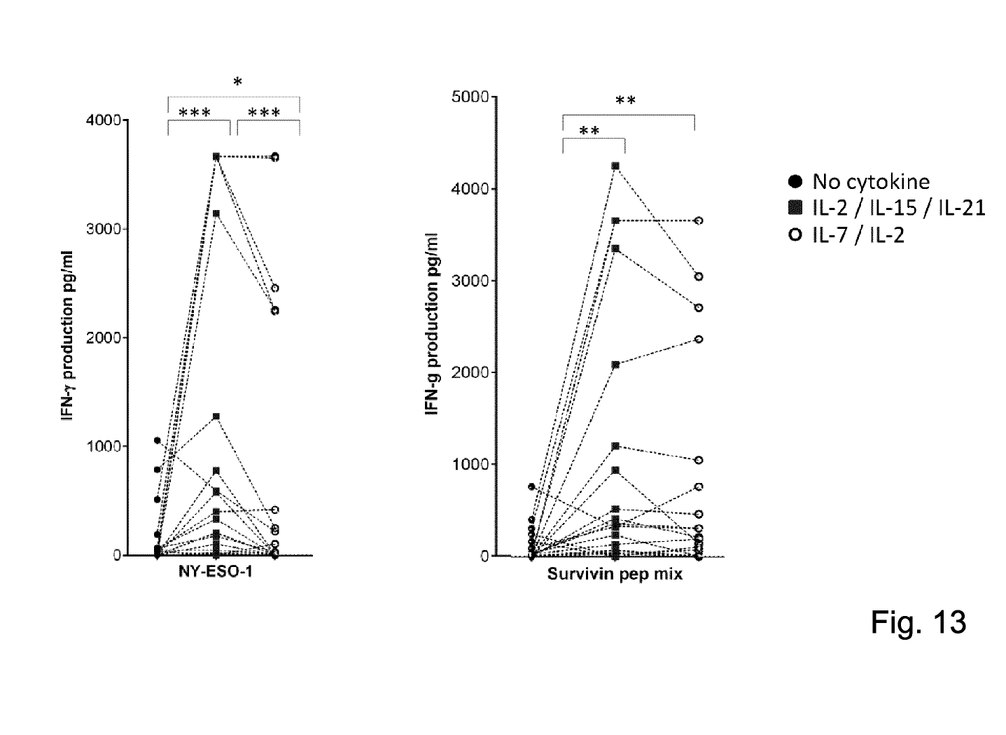

The experiment according to Example 20 was repeated with the viral antigens EBNA-1, EBNA-3a and CMVpp65. The results are summarized in FIG. 14. FIG. 14 shows strongly increased IFN-γ production and thus concentration of antigen-specific lymphocytes after expansion with IL-2, IL15 and IL-21 in comparison to the other cytokine composition or no cytokines.

### Example 22

### Analysis of Treg Expansion with the Cytokine Cocktail

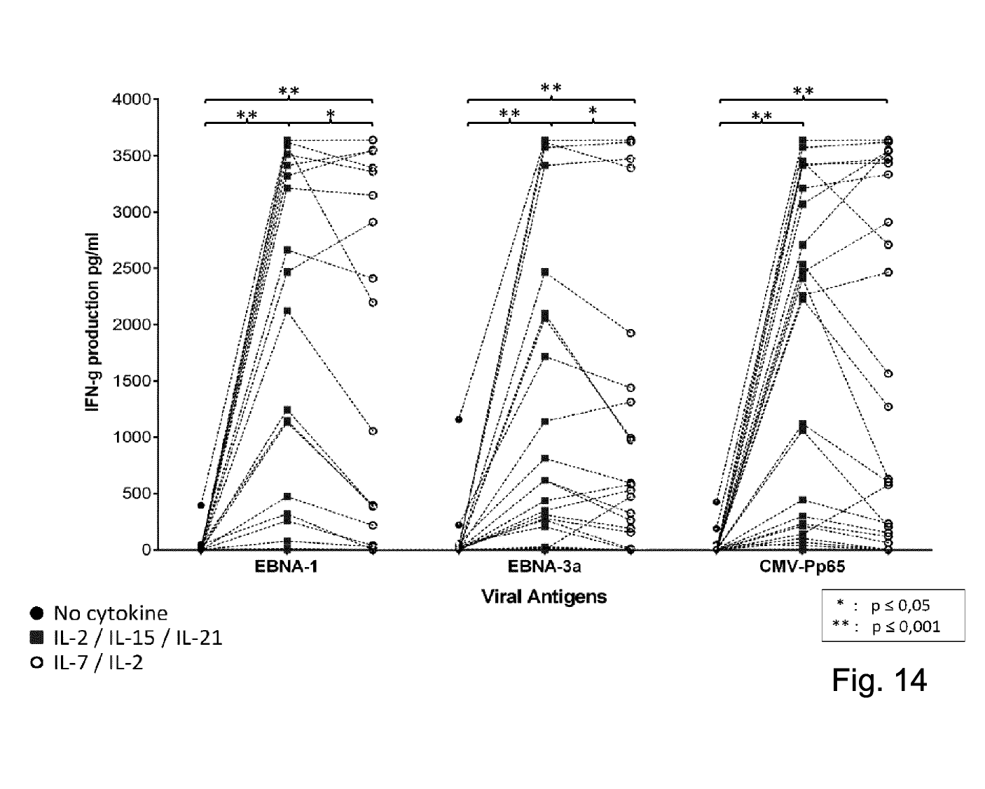

T-cells derived from PBMCs were cultured in the presence of the cytokine cocktail IL-2, IL-15 and IL-21 and Treg (regulatory T-cells) were identified prior to and after expansion of T-cells using flow cytometry. The results are shown in FIG. 15. In FIG. 15 from left to right: the gating of the T-cells on CD4+ T-cells and then on CD25high designating the high expression of the IL-2 receptor on activated T-cells is shown. Then the cells were gated on II-2R (high CD125) cells and tested for expression of the IL-7Receptor (CD127) and Foxp3 (intracellularly). A Treg cell is defined as CD4+CD25high, Foxp3+ and CD127-negative. The number of Treg was initially low (0.07% in CD4+ T-cells) and was even lower after T-cell expansion (0.01%). The data imply that the cytokine cocktail is superior in T-cell expansion since IL-2 is known to drive strong Treg expansion, it also underlines the synergistic effects of the IL-2/IL-15/IL-21 combination that blocks Treg expansion, while allowing anti-tumor directed T-cells to expand.

### Example 23

### Analysis of VB Family Distribution after Expansion

T-cells were expanded using the cytokine mix IL-2/IL-15/IL-21 along with the NY-ESO-1 antigens for 21 days. A flow cytometric analysis has been performed before (T0=time point 0) and after expansion (TH=Time of harvest). T-cells stimulated with cytokines alone served as the control. The results are summarized in Table 9. Note the strong preferential expansion of the VB7.2 family in the CD8+ T-cells expanded in the GRex flask, as well as the VB13.2 family in the CD8+ T-cells (also expanded in the GRex flask). The data show that the presence of the antigen (NY-ESO-1) drives preferential expansion of individual TCR VB families—and shows also the diversity of the response driven by the antigen (not only by the cytokines). Focused, yet diverse TCR VB families suggest a diverse anti-target directed T-cell response.

TABLE 9
TH: Harvest timepoint, 21 days; G-Rex: G-Rex flask, NY-ESO-1
stimulation; Cyto: Cytokine only, no stimulation; GE: GE wave system,
NY-ESO-1 stimulation

| | |
|---|---|
| CD4+ | CD8+ |

| | | T 0 | TH | T 0 | TH |
|---|---|---|---|---|---|
| Vβ1 | G-Rex | 1.14 | 0.23 | 0.96 | 0.54 |
| | Cyto | | 1.71 | | 1.68 |
| | GE | | 1.18 | | 1.01 |
| Vβ2 | G-Rex | 6.74 | 0.57 | 3.54 | 1.55 |
| | Cyto | | 1.68 | | 5.06 |
| | GE | | 7.35 | | 0.10 |
| Vβ3 | G-Rex | 3.84 | 0.13 | 19.00 | 10.70 |
| | Cyto | | 1.35 | | 10.70 |
| | GE | | 1.15 | | 6.05 |
| Vβ4 | G-Rex | 1.68 | 0.20 | 4.53 | 1.30 |
| | Cyto | | 0.61 | | 3.92 |
| | GE | | 5.89 | | 4.78 |
| Vβ5.1 | G-Rex | 2.51 | 0.94 | 1.10 | 0.22 |
| | Cyto | | 3.91 | | 0.62 |
| | GE | | 0.98 | | 2.78 |
| Vβ5.2 | G-Rex | 0.37 | 0.13 | 0.18 | 0.10 |
| | Cyto | | 0.43 | | 0.17 |
| | GE | | 2.06 | | 0.30 |
| Vβ5.3 | G-Rex | 0.48 | 0.14 | 0.50 | 3.30 |
| | Cyto | | 0.83 | | 1.77 |
| | GE | | 2.58 | | 3.82 |
| Vβ7.1 | G-Rex | 5.47 | 79.30 | 0.66 | 0.53 |
| | Cyto | | 31.60 | | 0.96 |
| | GE | | 37.20 | | 1.19 |
| Vβ7.2 | G-Rex | 2.00 | 0.90 | 4.39 | 17.50 |
| | Cyto | | 3.46 | | 4.62 |
| | GE | | 5.84 | | 8.46 |
| Vβ8 | G-Rex | 2.83 | 0.38 | 1.39 | 0.63 |
| | Cyto | | 2.58 | | 2.92 |
| | GE | | 1.64 | | 5.38 |
| Vβ9 | G-Rex | 1.64 | 0.20 | 1.84 | 2.83 |
| | Cyto | | 1.14 | | 1.59 |
| | GE | | 3.45 | | 0.57 |
| Vβ11 | G-Rex | 0.45 | 0.11 | 1.09 | 0.25 |
| | Cyto | | 0.31 | | 4.10 |
| | GE | | 0.13 | | 0.33 |
| Vβ12 | G-Rex | 0.68 | 0.12 | 3.09 | 0.33 |
| | Cyto | | 0.43 | | 1.00 |
| | GE | | 2.31 | | 0.44 |
| Vβ13.1 | G-Rex | 7.42 | 4.75 | 1.96 | 1.16 |
| | Cyto | | 2.65 | | 0.93 |
| | GE | | 16.90 | | 1.90 |
| Vβ13.2 | G-Rex | 4.56 | 2.65 | 14.70 | 28.70 |
| | Cyto | | 2.10 | | 18.50 |
| | GE | | 16.70 | | 12.40 |
| Vβ13.6 | G-Rex | 0.85 | 0.07 | 0.71 | 6.30 |
| | Cyto | | 0.79 | | 0.76 |
| | GE | | 8.34 | | 10.00 |
| Vβ14 | G-Rex | 15.30 | 1.50 | 11.30 | 4.83 |
| | Cyto | | 8.17 | | 8.45 |
| | GE | | 15.50 | | 0.04 |
| Vβ16 | G-Rex | 0.53 | 0.08 | 0.33 | 0.09 |
| | Cyto | | 0.20 | | 0.05 |
| | GE | | 3.38 | | 0.72 |
| Vβ17 | G-Rex | 2.89 | 0.27 | 1.06 | 0.54 |
| | Cyto | | 1.15 | | 1.33 |
| | GE | | 7.07 | | 0.14 |
| Vβ18 | G-Rex | 0.81 | 0.75 | 0.29 | 0.03 |
| | Cyto | | 2.05 | | 0.12 |
| | GE | | 1.61 | | 1.77 |
| Vβ20 | G-Rex | 0.95 | 0.42 | 0.74 | 1.78 |
| | Cyto | | 1.37 | | 1.33 |
| | GE | | 1.17 | | 1.24 |
| Vβ21.3 | G-Rex | 3.70 | 2.94 | 0.32 | 0.31 |
| | Cyto | | 3.07 | | 0.66 |
| | GE | | 3.86 | | 0.55 |
| Vβ22 | G-Rex | 2.97 | 0.22 | 1.03 | 0.12 |
| | Cyto | | 1.38 | | 0.58 |
| | GE | | 0.51 | | 0.02 |
| Vβ23 | G-Rex | 0.20 | 0.93 | 0.34 | 0.58 |
| | Cyto | | 3.06 | | 2.17 |
| | GE | | 7.42 | | 12.30 |

### Example 24

### PD1 Marker in CD8+ Cells

T-lymphocytes have been expanded using the IL-2/IL-15/IL-21 and the NY-ESO-1 peptide mix. Top panel: prior, bottom panel: after cytokine/TAA expansion=T-cells have been cultured in the presence of IL-2/IL-15/IL-21. A flow cytometic analysis has been performed before and after T-cell stimulation. From Left to right: i) gating on lymphocytes, ii) gating on CD3+ T-cells. iii) gating on CD4/CD8. Top right: PD1 expression on CD8+ activated T-cells (37% T-cells). Cytokine/TAA-expanded T-cells show decreased PD1 expression on the CD4CD8+ T-cells. The data show that the cytokine/peptide expanded T-cells exhibit decreased frequency of PD1+ T-cells on CD8+ T-cells—that implies that these expanded T-cells may show longer life-time and therefore superior efficacy against tumor cells.

### Example 25

### Multiple Antigens

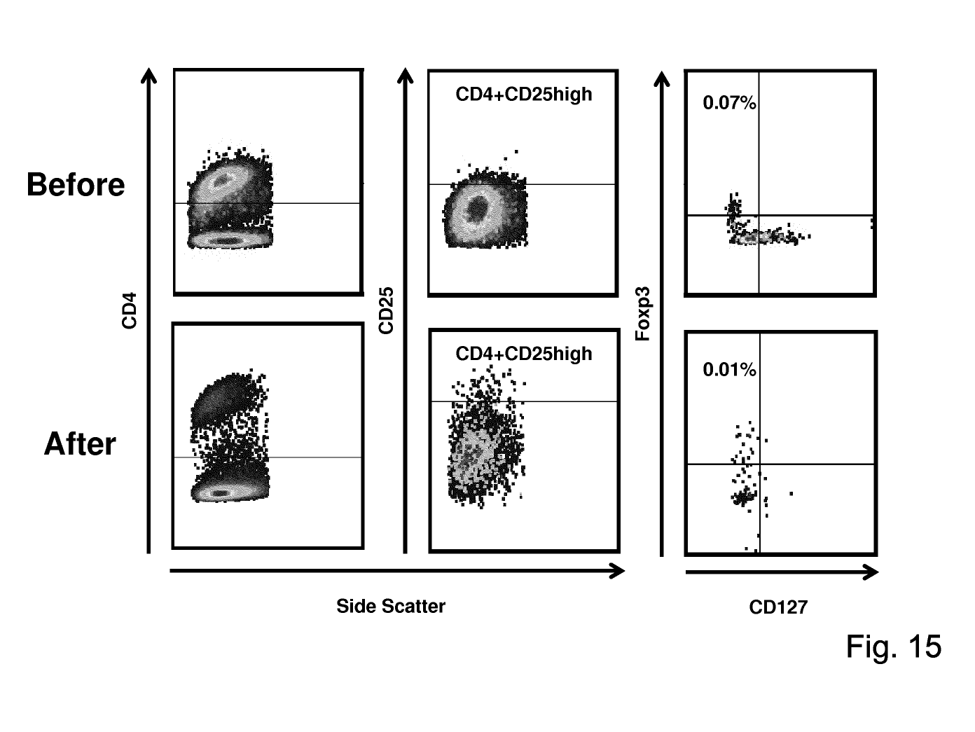

A peptide cocktail consisting of 12 individual peptides from the L1 protein from HPV 33, along with the cytokine cocktail, was used to stimulate T-cells from patients with HPV+ lesions. At day 21, the T-cells were tested for cytotoxicity directed against each individual peptide. Here, autologous EBV-transformed B-cells were taken and pulsed with peptides 1-12, followed by a standard Cr51 assay. This assays measure the capacity of T-cells to kill specific targets. The results are shown FIG. 16.

T-cells from donor A recognize strongly peptides 11 and 12. T-cells from d B reccognized strongly peptides 7-12 with a strong reactivity against peptide 11. These data show that expansion of T-cells with the IL-2/IL-15/IL-21 cytokine cocktail and peptides leads to i) generation of cytotoxic T-cells and ii) that the T-cell response is diverse and focused to a variant set of individual peptide species. This implies that T-cells stimulated with the peptide/cytokine cocktail can be preferentially cytotoxic and they can also target several epitopes—which makes them more diverse in recognizing tumor cells, or virally infected cells, that display on their surface different sets of peptide species.

### Example 26

### CD117 Expression Before and After Cytokine- and NY-ESO-1 Driven Expansion of PBMCs.

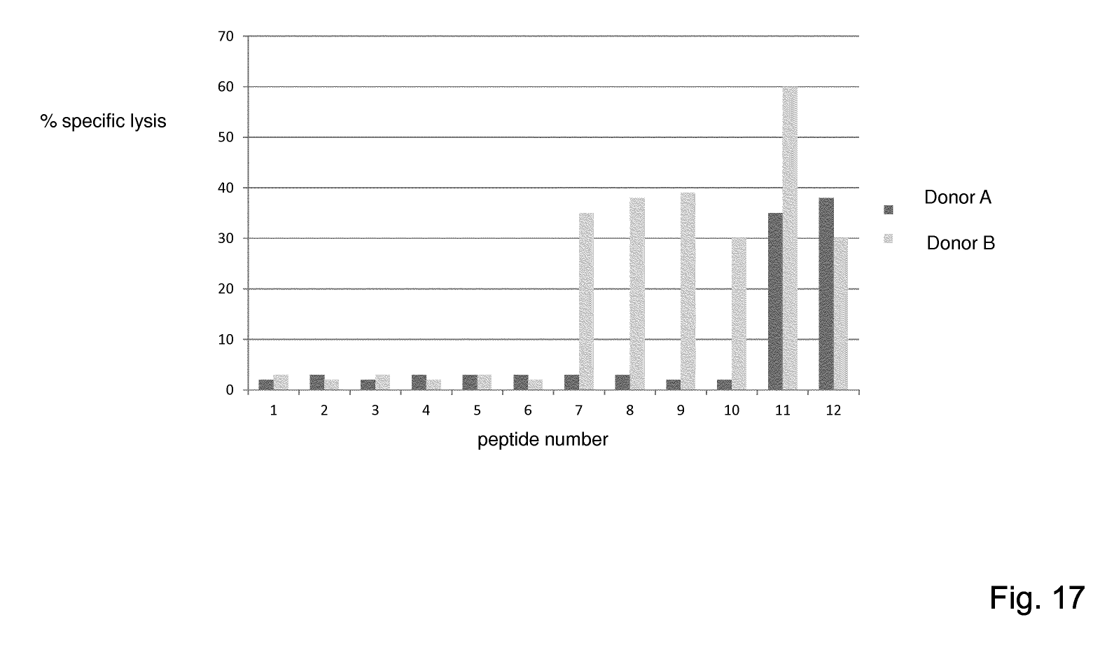

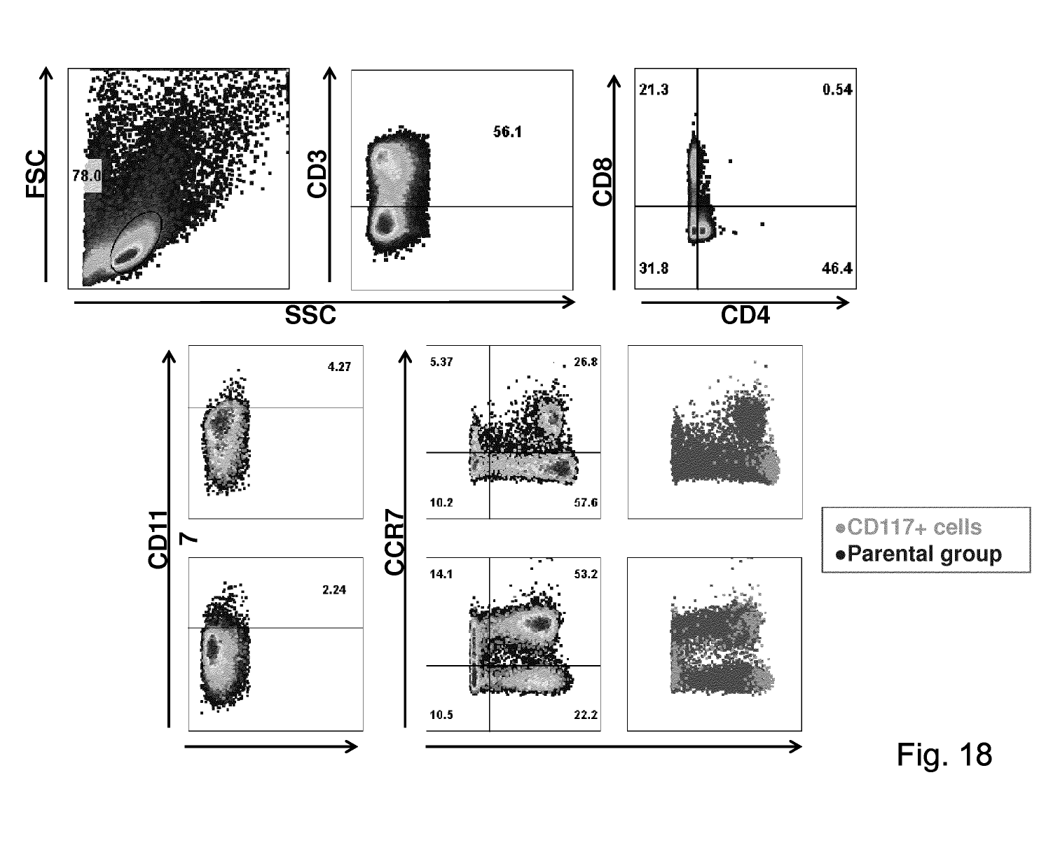

PBMCs were expanded with IL-2/L-15/L-21-mix in the presence of the tumor-associated antigen NY-ESO-1. The cells were analyzed by flow cytometry before (FIG. 18) and after expansion (FIG. 19). First, CD3+ T-cells are gated, then the CD3+ T-cells are gated on CD4+ and CD8+ T-cells. CD117+ cells were then detected on CD8+ T-cells (top) and on CD4+ T-cells (bottom panel, left). Middle panel: CD45RA/CCR7 expression in CD8+ and CD4+ T-cells with a high population on CD45RA+ CCR7+ T-cells, representing precursor Tcells (middle panel). Right: CD117+ T-cells (blue labeled) reside in effector T-cells in CD8+ T-cells; CD117+ T-cells can be found in different T-cell populations in the CD4+ T-cell subsets. The data implies that CD117 expression, a marker of T-cell ‘stem-ness’ present is present on different T-cell subsets. CD117+ T-cells are advantageous to provide a source for long-term T-cell memory.

First, CD3+ T-cells are gated, then the CD3+ T-cells are gated on CD4+ and CD8+ T-cells. Very strong expression and a high frequency of CD117+ cells on

CD8+ T-cells (top) and on CD4+ T-cells (bottom panel, left). Middle panel: CD45RA/CCR7 expression in CD8+ and CD4+ T-cells with a high population on CD45RA+ CCR7+ T-cells, representing precursor T cells (middle panel). Right: CD117+ T-cells (blue labeled) reside in periphery effector T-cells in CD45RA+CCR7− T-cells (effectors). CD84 T-cells; CD117+ T-cells can be found in the T-cell precursor CD45RA+CCR7+ population. The data implies that CD117 expression, a marker of T-cell ‘stem-ness’ present is present on different T-cell subsets. CD117+ T-cells are advantageous to provide a source for long-term T-cell memory. It also shows that the cytokine cocktail strongly expands CD117+ T-cells that bring about long-term immune cell memory, also that CD117+ T-cells reside in precursor T-cells—to ensure long-term tumor immune responses.

### Example 27

### Expansion of TIL Culture from Glioblastoma

This example describes the procedure for culturing TILs from pancreatic cancer for a function test and immunotherapy.

For material, equipment and supplies see Example 1.

Tumor tissue obtained from a patient was put into a sterile container. The tissue was cut into pieces of 1 to 2 mm³ with a sterile scalpel. The tissue pieces were put into the wells of 24 well plate with 1 piece per well. Cell culture medium is prepared using Cellgro with 10% human AB serum. Into this medium IL-2, IL-15 and IL-21 are added to a final concentration of 1000 u/ml for IL-2, 10 ng/ml for each of IL-15 and IL-21. Furthermore, PEST and amphotericin were added to the medium. 1 ml medium was added into each well and the cell culture was incubated for 7 days at 37° Celsius. In parallel PBMCs from a healthy donor were cultured in Cellgro containing 10% human AB serum. The concentration of the PBMCs was determined and adjusted to a concentration of 10⁶/ml. The PBMCs were then radiated for 18 minutes at 55 Gy. After Irradiation OKT3 was added to a final concentration of 10 ng/ml. This culture is referred to as feeder cells with OKT3. On day 10 of the TIL culture, 100 μl of the feeder cell culture with OKT3 is added to each of the wells of the 24 well plate. Accordingly, the culture in each of the well to the final concentration of 10 ng/ml OKT3 and a total amount of 10⁶ feeder cells. The ratio of TILs and feeder cells is about 1:10.

### Example 28

### Expansion of TIL Culture from Pancreatic Cancer

Tumor tissue obtained from a patient was put into a sterile container. The tissue was cut into pieces of 1 to 2 mm³ with a sterile scalpel. The tissue pieces were put into the wells of 24 well plate with 1 piece per well. Cell culture medium is prepared using Cellgro with 10% human AB serum. Into this medium IL-2, IL-15 and IL-21 are added to a final concentration of 1000 u/ml for IL-2, 10 ng/ml for each of IL-15 and IL-21. Furthermore, PEST and Amphotericin were added to the medium. 1 ml medium was added into each well and the cell culture was incubated for 4 days at 37° Celsius. In parallel PBMCs from a healthy donor were cultured in Cellgro containing 10% human AB serum. The concentration of the PBMCs was determined and adjusted to a concentration of 10⁶/ml. The PBMCs were then radiated for 18 minutes at 55 Gy. After Irradiation OKT3 was added to a final concentration of 10 ng/ml. This culture is referred to as feeder cells with OKT3. On day 10 of the TIL culture 100 pl of the feeder cell culture with OKT3 is added to each of the wells of the 24 well plate. Accordingly, the culture in each of the well contains the final concentration of 10 ng/ml OKT3 and a total amount of 10⁶ feeder cells. The ratio of TILs and feeder cells is about 1:10.

### Example 29

### Procedure for Generating a Tumor Cell Line from Tissue

Pancreas tumor tissue is obtained directly after operation and put into a sterile container. The tumor tissue is cut into pieces of 1 to 2 mm³ with a sterile scalpel. Each of the tissue pieces are to be transferred into a well of a 24 well plate. 1 ml of the culture medium RPMI 1640 with 10% fetal bovine serum was added into each of the wells. The medium was changed on day 7 and day 14 which means that the culture medium was removed and replaced by fresh culture medium of the same kind. When the tumor cells reached a very high density the culture was transferred to a 6 well plate.

### Example 30

### Determination of the Correct Time Point for Feeder Cell Addition

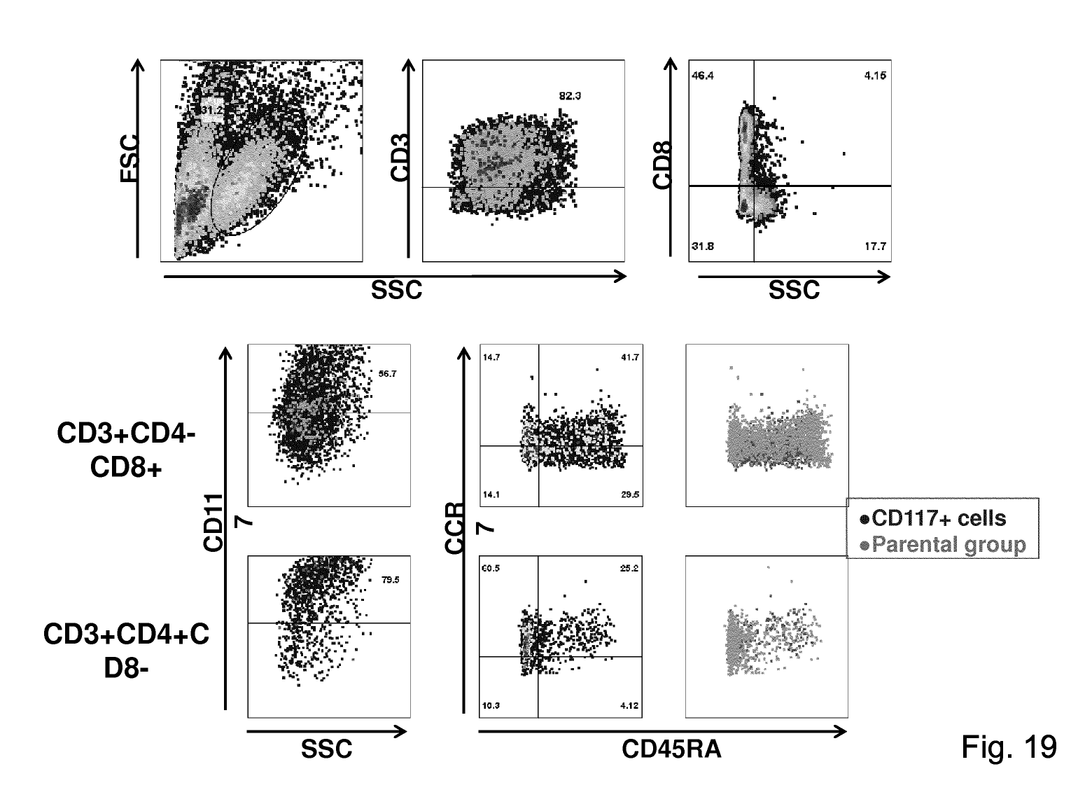

It was found that the addition of feeder cells does not lead to a good expansion of tumor infiltrating lymphocytes if the feeder cells are added when the concentration of lymphocytes is very low. FIG. 20A shows the lymphocyte culture of the one week of incubation. Some lymphocytes are detectable, however the concentration of lymphocytes is below the basic line of expansion. FIG. 20B and FIG. 20C show the same lymphocyte culture after two weeks of incubation. Now the number of lymphocytes that can be detected has increased in this above the basic line of expansion. Feeder cells may be added at this stage. FIGS. 1D, E and E show the culture four days, one week and two weeks subsequently after adding the feeder cells. In the figures can be seen that the number of lymphocytes drastically increases from the image in FIG. D to the image in FIG. F. In FIG. F it can be seen that the lymphocytes are gathering and attacking tumor cells in culture.

### Example 31

### Analysis of Phenotype and Activation/Exhaustion Marker Expression of Lymphocytes Expanded from TIL Obtained from Glioblastoma

Tumor tissue was obtained from 16 Glioblastoma patients. TILs were expanded from the tissue with the cytokine cocktail of IL-2, IL-15 and IL-21 according to the protocol of Example 1.

A)

The expanded cells were analyzed by flow cytometric analysis with regard to their CD4/CD8 phenotype using antibodies directed against CD3, CD4 and CD8.

The results are summarized in Table 10.

TABLE 10

| | | | | Tumor | | | | CD4− | CD4+ |
|---|---|---|---|---|---|---|---|---|---|
| | Age | M7F | Diagnosis | cell line | CD3+ | CD8+ | CD4+ | CD8− | CD8+ |
| GBM-A | 69 | M | GBM (grade IV) | Y | 75.50 | 11.40 | 85.20 | 2.92 | 0.47 |
| GBM-B | 66 | M | PXA (grade II-III) | Y | 73.00 | 90.10 | 8.31 | 1.07 | 0.50 |
| GBM-C | 64 | M | GBM (grade IV) | Y | 94.60 | 12.40 | 85.40 | 2.13 | 0.25 |
| GBM-D | 65 | F | GBM (grade IV) | Y | 73.50 | 24.20 | 71.20 | 3.71 | 0.99 |
| GBM-E | 54 | M | GBM (grade IV) | Y | 98.20 | 4.00 | 89.90 | 2.63 | 3.53 |
| GBM-F | 70 | M | GBM (grade IV) | Y | 99.60 | 3.58 | 89.90 | 6.15 | 0.38 |
| GBM-G | 49 | M | GBM (grade IV) | Y | 94.30 | 0.52 | 77.80 | 21.60 | 0.08 |
| GBM-H | 76 | M | GBM (grade IV) | Y | 92.70 | 38.50 | 52.60 | 5.80 | 3.10 |
| GBM-I | 45 | M | GBM (grade IV) | Y | 98.50 | 44.20 | 2.81 | 52.90 | 0.08 |
| GBM-J | 73 | F | GBM (grade IV) | Y | 98.00 | 1.16 | 91.70 | 6.34 | 0.84 |
| GBM-K | 67 | M | GBM (grade IV) | Y | 85.20 | 33.30 | 49.40 | 10.80 | 6.50 |
| GBM-L | 40 | F | GBM (grade III) | Y | 94.20 | 81.30 | 12.20 | 6.02 | 0.46 |
| GBM-M | 79 | M | GBM (grade IV) | Y | 99.90 | 0.02 | 92.50 | 7.39 | 0.10 |
| GBM-N | 50 | F | O (grade II) | Y | 99.70 | 95.40 | 0.24 | 4.33 | 0.07 |
| GBM-O | 17 | M | AE (grade III) | Y | 82.50 | 0.59 | 84.70 | 14.60 | 0.12 |
| GBM-P | 42 | F | Relapse Necrosis | Y | 99.40 | 1.37 | 80.70 | 17.80 | 0.10 |
| N = 16 | 58 | 11M/4F | | Y | | | | | |
| | (17-79) | | | 100% | | | | | |

The data show that TIL could be expanded from each individual tumor mainly contain CD3+ T-cells. Depending on the patient either one of CD4+, CD8+ and DN T-cell are in the majority. However, CD4+ was found most frequently in majority. The table also shows that the cytokine cocktail is able to expand T-cells from different CNS tumor histologies. (see column 4 “Diagnosis”)

B)

The expanded cells were analyzed by flow cytometric analysis with regard to their specific phenotype-precursor (CD45RA+ CCR7+), central memory (CD45RA−CCR7+), peripheral memory (CD45RA−CCR7−), or differentiated effector (CD45RA+CCR7−) T-cells and the expression of activation/exhaustion markers in the base phenotypes CD8+, CD4+ and double negative T-cells.

The antibodies used for flow cytometric analysis of TIL populations are summarized in Table 11.

TABLE 11

| Immuno- | | |
|---|---|---|
| phenocyte | Color | Clone |
| CD3 | PerCp | SK7 |
| CD4 | krome Orange | 13B8.2 |
| CD8a | APC Cy7 | SK1 |
| CD8b | FiTC | 2ST8.5H7 |
| CD107a | Alexa Fluor 700 | H4A3 |
| CD45RA | ECD | 2H4 |
| c-kit | PE Cy7 | 104D2 |
| CD127 | APC | R34.34 |
| CCR7 | Brilliant violet 421 | G043H7 |
| Exhaustion and | | |
| Activation | Color | Clone |
| CD3 | Brilliant violet 570 | UCHT1 |
| CD4 | Brilliant violet 510 | OKT4 |
| CD8a | APC Cy7 | SK1 |
| 4-1BB | FITC | 4B4-1 |
| CD127 | APC AF700 | R34.34 |
| CD45RA | ECD | 2H4 |
| CCR7 | Brilliant violet 421 | G043H7 |
| LAG-3 | APC | polyclonal |
| CD25 | PE Cy7 | 2A3 |
| CTLA-4 | PE Cy5 | BNI3 |
| TIM3 | PercP eFluor 710 | F38-2E2 |
| PD-1 | PE | EH12.1 |
| Intracellular | | |
| Cytokine | | |
| Staining | Color | Clone |
| CD3 | ECD | UCHT1 |
| CD4 | PerCp Cy5.5 | L200 |
| CD8a | APC Cy7 | SK1 |
| TNF-α | APC | MAb11 |
| IFN-γ | PE Cy7 | B27 |
| IL-2 | PE | MQ1-17H12 |
| Vβ Repertoire | Color | Clone |
| CD3 | PE Cy7 | SK7 |
| CD4 | krome Orange | 13B8.2 |
| CD8a | APC Cy7 | SK1 |
| TCR Vβ | | |
| Repertoire Kit | | |
| Treg | Color | Clone |
| CD3 | ECD | UCHT1 |
| CD4 | V450 | RPA-T4 |
| CD8a | APC Cy7 | SK1 |
| CD25 | PE Cy7 | 2A3 |
| CD127 | APC | R34.34 |
| CD73 | efluor 710 | AD2 |
| CD39 | PE | TU66 |
| FoXP3 | Alexa fluor 488 | 259D |

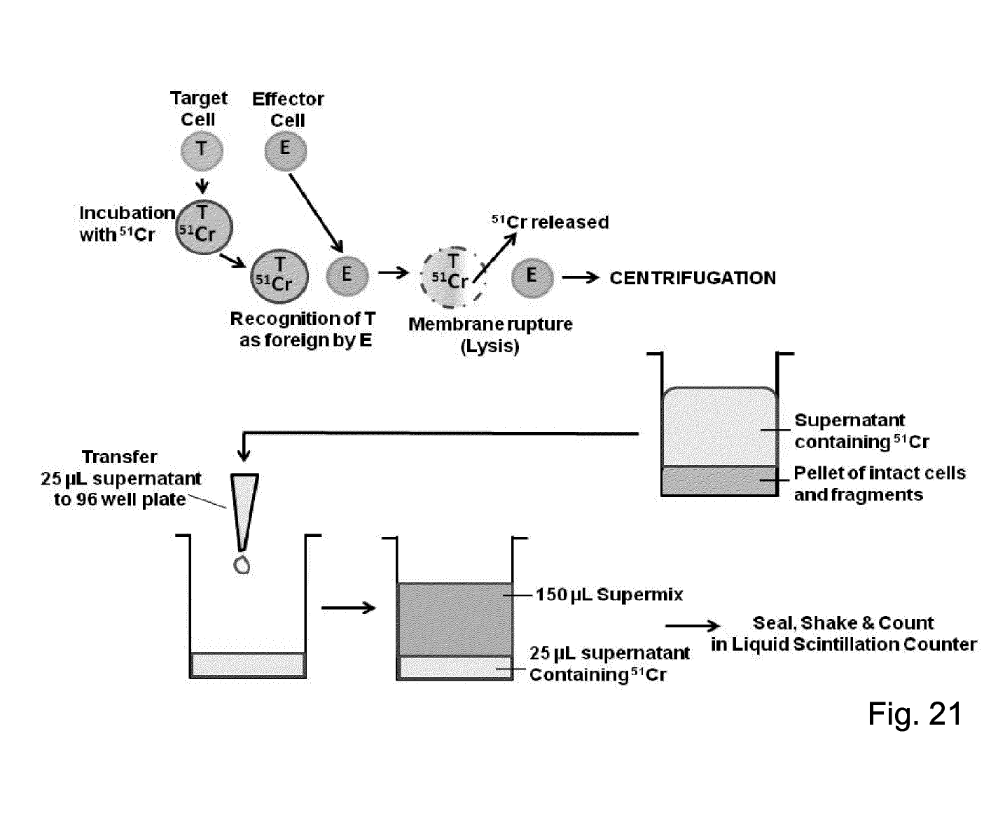

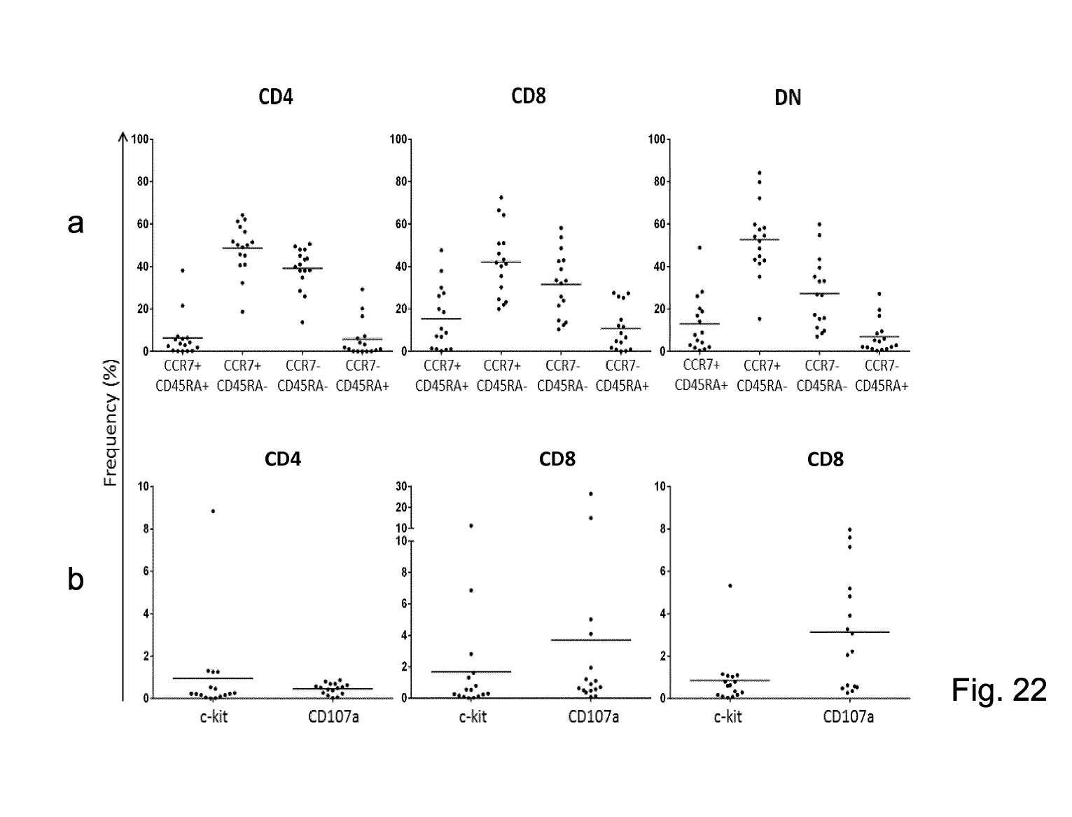

The results are summarized in FIG. 22A and B. The majority of CD8+, CD4+ or DN T-cells are in the central memory T-cell subset that has been shown to be crucial for effective anti-cancer responses and long-term immune memory. The results shows in average a strong expansion of DN-T-Cells. This subset is highly activated and bears affinity T-cell receptors. Also a strong expansion of CD45RA+CCR7+ precursor T-cell subsets is observed that provide long-term memory T-cell responses advantageous for long-term immune surveillance.

The reported expression of 4-1BB, LAG-3 or TIM-3 in the T-cells (see FIG. 3B) are indicative for antigen/tumor specific T-cells. The example shows that the cytokine cocktail expands TIL that reside primarily in the memory (the central memory) T-cell subset which has been associated with beneficial clinical responses, it also shows that TIL are expanded that bear markers associated with antigen-specificity.

### Example 32

### Analysis of Phenotype and Activation/Exhaustion Marker Expression of Lymphocytes Expanded from TIL Obtained from Pancreas Cancer

Tumor tissue was obtained from 17 pancreas cancer patients. Table 12 summarizes the patient's age, sex, type of sample and histology.

TABLE 12
The cytokine cocktail expands TIL from different
pancreas cancer histologies.

| Patients ID | Age | Sex | Sample | Histology |
|---|---|---|---|---|
| Panc 1 | 72 | M | Biopsy | papillary adenocarcinoma |
| Panc 2 | 66 | M | Tumor | ductal adenocarcinoma |
| Panc 3 | 68 | M | Tumor | duodenal adenocarcinoma |
| Panc 4 | 67 | M | Tumor | ductal adenocarcinoma |
| Panc 5 | 81 | F | Tumor | ductal adenocarcinoma |
| Panc 6 | 50 | M | Biopsy | ductal adenocarcinoma |
| Panc 7 | 68 | M | Biopsy | ductal adenocarcinoma |
| Panc 8 | 74 | F | Tumor | ductal adenocarcinoma |
| Panc 9 | 71 | M | Tumor | ductal adenocarcinoma |
| Panc 10 | 60 | F | Tumor | adenocarcinoma |
| Panc 11 | 42 | F | Tumor | ductal adenocarcinoma |
| Panc 12 | 70 | M | Tumor | ductal adenocarcinoma |
| Panc 13 | 59 | F | Tumor | Pancreatic adenosquamous |
| | | | | carcinoma |
| Panc 14 | 60 | F | Tumor | ductal adenocarcinoma |
| Panc 15 | 72 | M | Tumor | ductal adenocarcinoma |
| Panc 16 | 81 | F | Tumor | ductal adenocarcinoma |
| Panc 17 | 61 | M | Biopsy | ductal adenocarcinoma |

TILs were expanded from the tissue with the cytokine cocktail of IL-2, IL-15 and IL-21 according to the protocol of Example 28. CD4/CD8 phenotype using antibodies directed against CD3, CD4 and CD8.

A)

The expanded cells were analyzed by flow cytometric analysis with regard to their CD4/CD8 phenotype using antibodies directed against CD3, CD4 and CD8. The results are summarized in Table 13.

TABLE 13
T-cell phenotype analysis in pancreas cancer TIL.
Dominant CD8+ TIL populations.

| | | | | CD4+ | CD4− |
|---|---|---|---|---|---|
| Patients ID | CD3+ | CD4+ | CD8+ | CD8+ | CD8− |
| Panc 1 | 99, 9 | 4, 33 | 89, 3 | 2, 33 | 4 |
| Panc 2 | 99, 9 | 56, 1 | 29, 8 | 2, 76 | 11, 3 |
| Panc 3 | 99, 8 | 17, 2 | 71, 4 | 1, 21 | 10, 1 |
| Panc 4 | 99, 6 | 50, 5 | 32, 1 | 2, 3 | 15, 1 |
| Panc 5 | 99, 5 | 99 | 0, 073 | 0, 51 | 0, 39 |
| Panc 6 | 99, 7 | 41, 1 | 51, 3 | 3, 09 | 4, 47 |
| Panc 7 | 99, 7 | 6, 57 | 89, 9 | 2, 62 | 0, 93 |
| Panc 8 | 99, 7 | 32, 8 | 64, 9 | 1, 09 | 1, 24 |
| Panc 9 | 94, 8 | 2, 07 | 97, 2 | 0, 3 | 0, 47 |
| Panc 10 | 98, 6 | 92, 2 | 5, 75 | 0, 6 | 1, 47 |
| Panc 11 | 99, 7 | 2, 24 | 96, 7 | 0, 3 | 0, 76 |
| Panc 12 | 99, 2 | 71, 8 | 22, 9 | 4, 12 | 1, 17 |
| Panc 13 | 91, 7 | 70, 9 | 24 | 3, 01 | 2, 06 |
| Panc 14 | 99, 9 | 0, 66 | 95, 7 | 0, 13 | 3, 55 |
| Panc 15 | 99, 1 | 75, 9 | 19, 5 | 3, 09 | 1, 47 |
| Panc 16 | 99, 3 | 34, 1 | 54, 4 | 8, 6 | 2, 86 |
| Panc 17 | 91, 8 | 17, 3 | 65, 1 | 2, 92 | 14, 6 |
| Mean | 99, 6 | 34, 1 | 54, 4 | 2, 33 | 2, 06 |

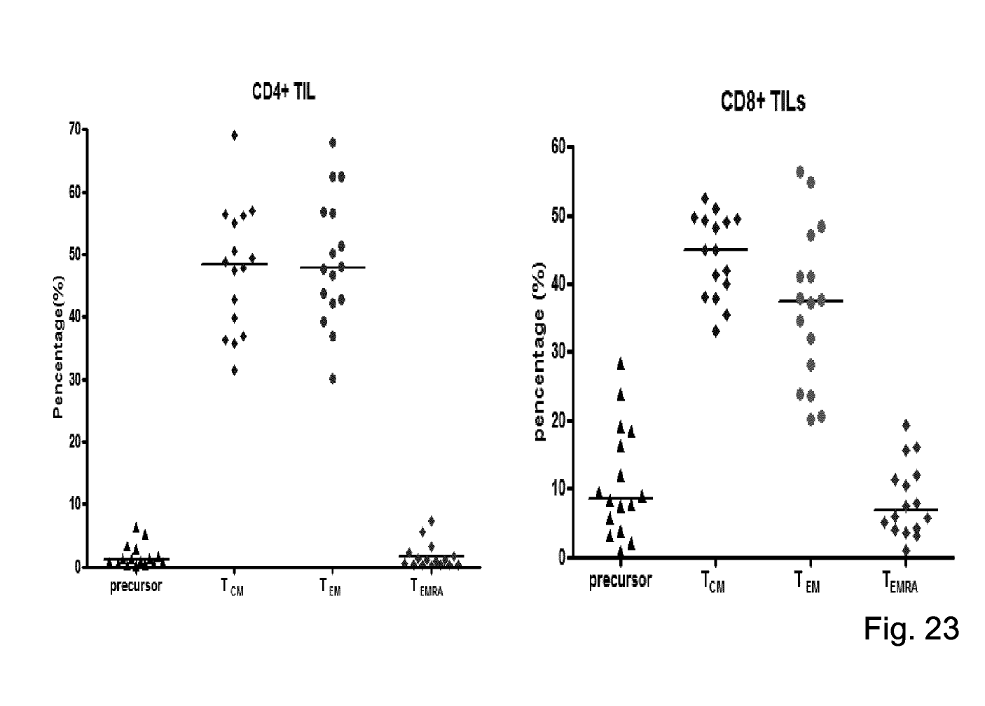

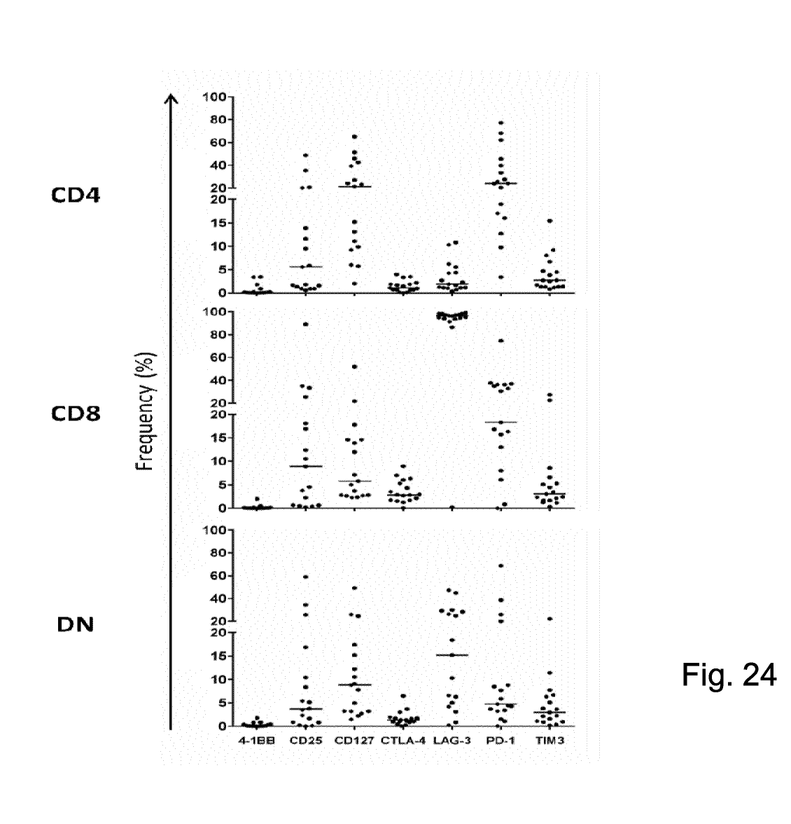

The expanded cells were analyzed by flow cytometric analysis with regard to their specific phenotype-precursor (CD45RA+CCR7+), central memory (CD45RA-CCR7+), peripheral memory (CD45RA−CCR7−), or differentiated effector (CD45RA+CCR7−) T-cells in the base phenotypes CD8+ and CD4+. The results are summarized in FIGS. 23 and 24.

The majority of CD8+, CD4+ or DN T-cells are in the central memory T-cell subset that has been shown to be crucial for effective anti-cancer responses and long-term immune memory. The results shows in average a strong expansion of DN-T-Cells (data not shown). This subset is highly activated and bears affinity T-cell receptors. Also a strong expansion of CD45RA+CCR7+ precursor T-cell subsets is observed that provide long-term memory T-cell responses advantageous for long- term immune surveillance.

The reported expression of 4-1BB, LAG-3 or TIM-3 in the T-cells (see FIG. 24) are indicative for antigen/tumor specific T-cells. The example shows that the cytokine cocktail expands TIL that reside primarily in the memory (the central memory) T-cell subset which has been associated with beneficial clinical responses, it also shows that TIL are expanded that bear markers associated with antigen-specificity.

### Example 33

### Analysis of the TCR Length of T-Cells Expanded from Tumor Tissue of Patients with Pancreatic Cancer

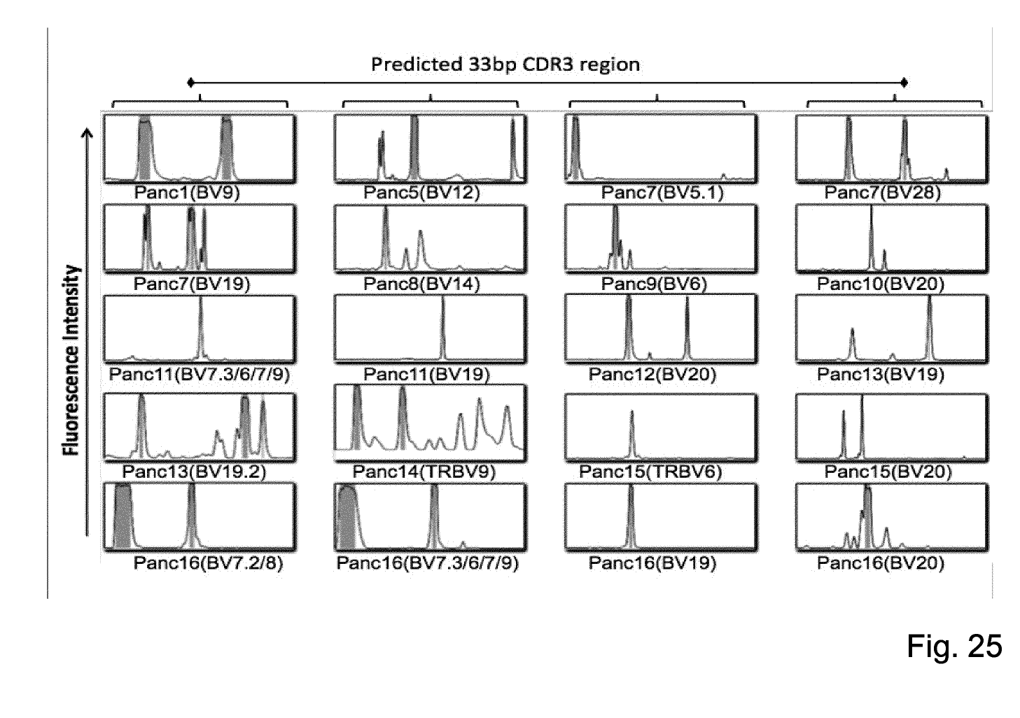

Tumor tissue was obtained from 17 pancreas cancer patients. TILs were expanded from the tissue with the cytokine cocktail of IL-2, IL-15 and IL-21 according to the protocol of Example 28. Using a PCR-based approach the TCR length was measured. The ‘normal’ image of a TCR family is a Gauss-distribution of the length of the T-cell receptor. Single peaks suggest monoclonality in individual TCR families. The data presented in FIG. 25 shows that the TIL composition is monoclonal or polyclonal, suggestive to be focused against autologous tumor targets, the cytokine cocktail aids to expand a focused TCR repertoire.

### Example 34

### Analysis of Cytokine Production in Lymphocytes Expanded from TIL Obtained from Patients with Glioblastoma

Tumor tissue was obtained from glioblastoma patients. TILs were expanded from the tumor tissue with the cytokine cocktail of IL-2, IL-15 and IL-21 according to the protocol of Example 1. The expanded lymphocytes were stimulated with peptides derived from tumor-associated antigens, i.e. the EGRvrIII, NY-ESO-1 or survivin and the percentage of cells producing either one of the cytokines IFNγ and TNFα was measured.

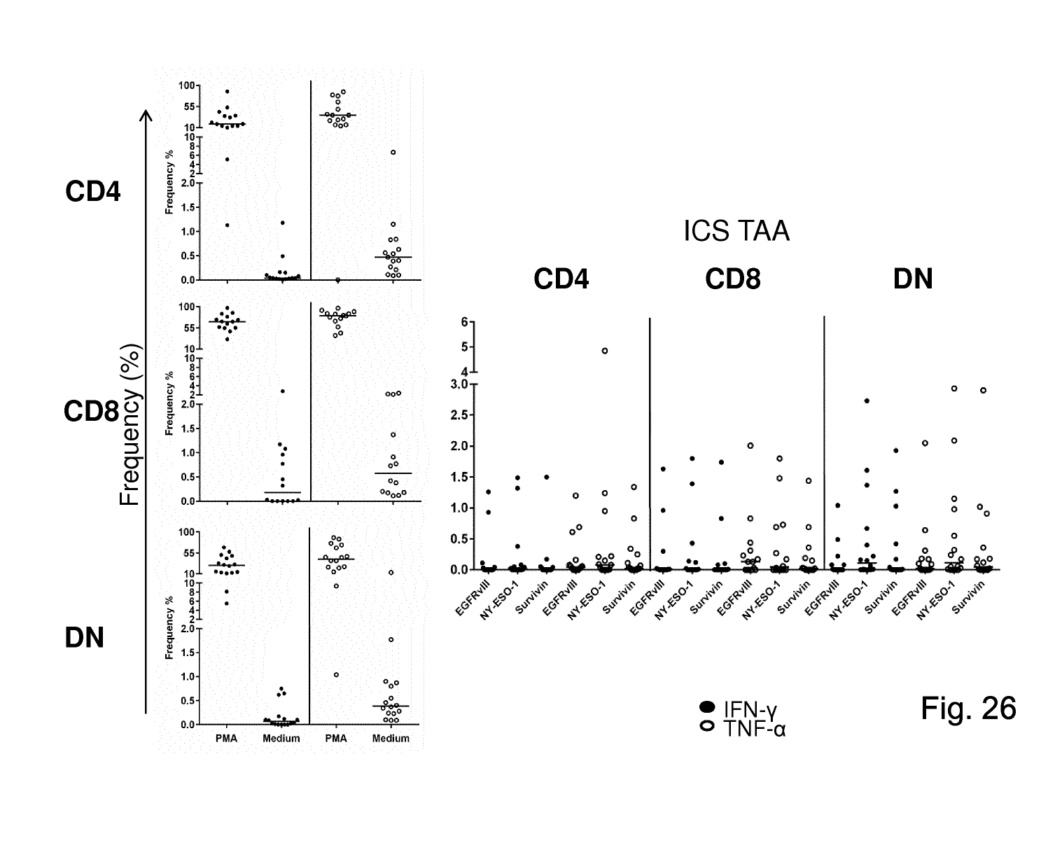

The results are shown in FIG. 26B. For comparison maximal stimulation is tested with PMA/inonomycin as a positive control and the background signal is determined with media only (negative control). The results demonstrate that the expanded T-cells recognize these commonly shared tumor antigens. Accordingly, the cytokine cocktail expands T-cells from glioblastoma that are reactive to tumor-antigens that have been described to be clinically relevant and associated with clinical responses.

### Example 35

### Analysis of Cytokine Production in Lymphocytes Expanded from TIL Obtained from Patients with Pancreas Cancer

Tumor tissue was obtained from pancreas cancer patients.

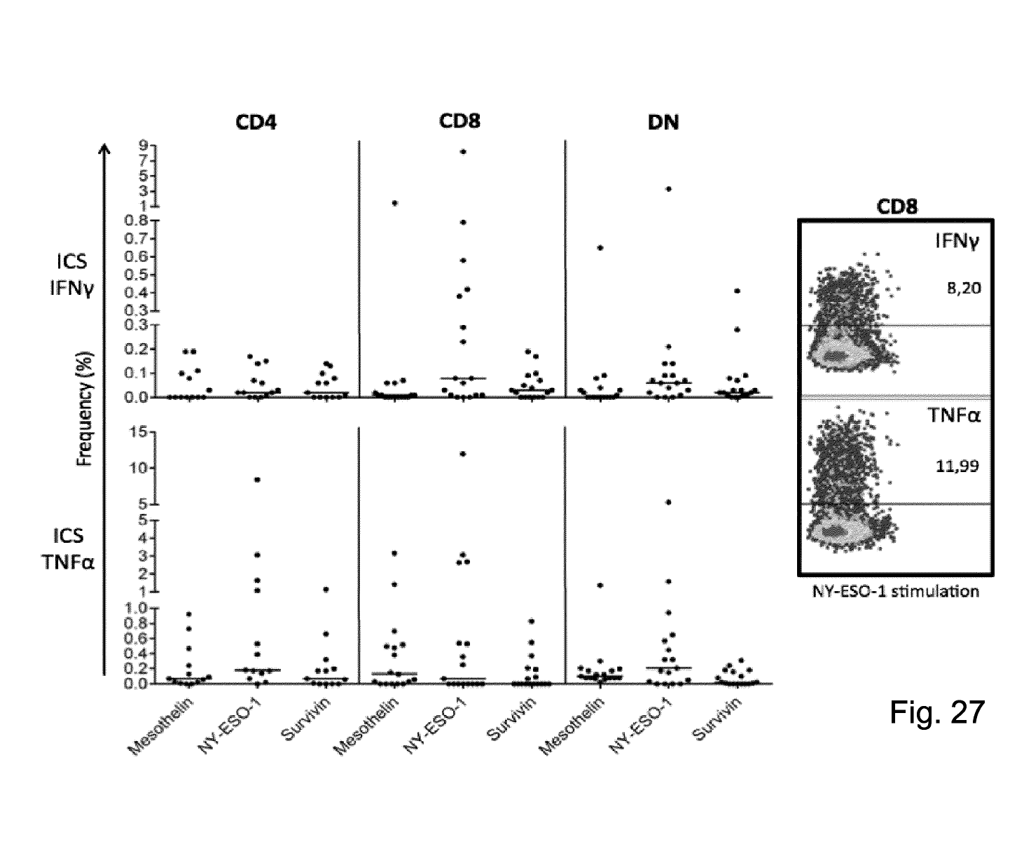

TILs were expanded from the tumor tissue with the cytokine cocktail of IL-2, IL-15 and IL-21 according to the protocol of Example 27. The expanded lymphocytes were stimulated with peptides derived from tumor-associated antigens, i.e. the EGRvrIII, NY-ESO-1 or survivin and the percentage of cells producing either one of the cytokines IFNγ and TNFα was measured. The results are shown in FIG. 27A. FIG. 27B shows images of the flow cytometry analysis for NY-ESO-1. The results confirm that the expanded T-cells recognize these commonly shared tumor antigens. Accordingly, the cytokine cocktail expands T-cells from pancreas tumor that are reactive to tumor-antigens that have been described to be clinically relevant and associated with clinical responses.

### Example 36

### Analysis of Cytokine Production in Lymphocytes Expanded from TIL Obtained from Patients with Glioblastoma and Autologous Stimulation

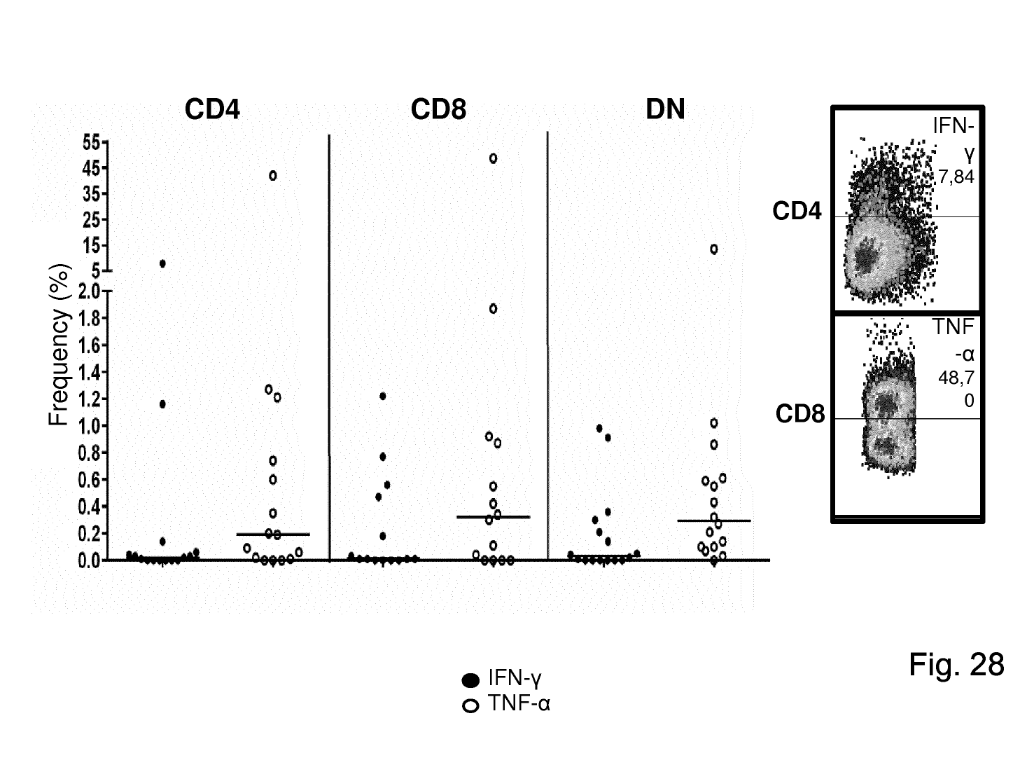

Tumor tissue was obtained from glioblastoma patients. TILs were expanded from the tumor tissue with the cytokine cocktail of IL-2, IL-15 and IL-21 according to the protocol of Example 27. The expanded lymphocytes were stimulated with autologous tumor cells. The results are shown in FIG. 28A. FIG. 28B shows images of the flow cytometry analysis. The results confirm that the expanded T-cells recognize autologous tumor cells. Accordingly, the cytokine cocktail expands T-cells from glioblastoma that are reactive to autologous tumor cells, accordingly the patient's own mutations.

### Example 37

### Link of TCR Usage in Pancreatic Cancer with Recognition of Autologous Tumor Cells

Tumor tissue was obtained from glioblastoma patients. TILs were expanded from the tumor tissue with the cytokine cocktail of IL-2, IL-15 and IL-21 according to the protocol of Example 27. The expanded lymphocytes were stimulated with autologous tumor cells. TIL were first gated on CD3+ T-cells, then on CD4+ and CD8+ T-cells. The frequency of individual Vβ families were tested using a panel of TCR VB antibodies. This TCR panel covers about 75% of the human TCR repertoire, there is therefore a possibility that not all TCR Vβ families are captured sufficiently. The TCR Vβ family distribution is about 2 to 6% in each family, except for TCR VB 2 that can reach over 10%. Table 14 shows the preferential expansion of the Vβ-2 family in TIL from patients with glioblastoma.

The data of Table 14 shows that individual TCR VB are preferentially expanded in TIL. It is noted that clonality can only be addressed by sequencing. It is noted that the cytokine cocktail expands different TCR Vβ families in individual patients. This is true for patients with glioblastoma, as well as for patients with patients with pancreatic cancer. Note also that some TIL are composed for a single or two VB families showing a highly focused TCR VB expansion. suggestive of an antigen-driven T-cell expansion process. Some TIL that we have shown to be preferentially expanded were shown to be monoclonal. Thus, the cytokine cocktail of IL-2, IL-15 and IL-21 expands a focused T-cell response, that is directed against the patient's own tumor cells.

### Example 38

### Link of TCR Usage in Pancreatic Cancer with Recognition of Autologous Tumor Cells

Tumor tissue was obtained from pancreatic cancer patients. TILs were expanded from the tumor tissue with the cytokine cocktail of IL-2, IL-15 and IL-21 according to the protocol of Example 28.

Cells were stained by flow cytometry using CD3, CD4 and CD8 in combination with a TCR Vβ antibody. The panel that covers up to 75% of the entire TCR VB repertoire. If certain T-cell families reside in the 25% that are not covered by this panel, they are not captured in this panel. The results are summarized in Table 15.

TABLE 15

| | Panc 1 | | Panc 2 | | Panc 3 | | Panc 4 | | Panc 5 | | Panc 6 | |
|---|---|---|---|---|---|---|---|---|---|---|---|---|
| | CD4 | CD8 | CD4 | CD8 | CD4 | CD8 | CD4 | CD8 | CD4 | CD8 | CD4 | CD8 |
| Vβ1 | 2.8 | 77.1 | 3.2 | 2.3 | 3.4 | 0.8 | 0.2 | 2.1 | 1.5 | 0.2 | 0.7 | 1.1 |
| Vβ2 | 8.0 | 0.6 | 9.8 | 2.6 | 4.8 | 0.2 | 8.5 | 2.0 | 0.0 | 0.0 | 9.6 | 19.9 |
| Vβ3 | 2.1 | 0.2 | 4.8 | 1.9 | 26.7 | 0.5 | 4.8 | 8.3 | 0.0 | 6.1 | 4.2 | 0.3 |
| Vβ4 | 0.4 | 0.0 | 0.0 | 0.0 | 0.1 | 0.0 | 0.0 | 0.0 | 0.0 | 0.0 | 0.0 | 0.0 |
| Vβ5.1 | 0.4 | 0.4 | 0.7 | 0.5 | 0.3 | 0.0 | 0.1 | 0.0 | 0.0 | 0.3 | 9.0 | 0.3 |
| Vβ5.2 | 0.2 | 0.1 | 1.5 | 0.0 | 1.5 | 0.0 | 0.1 | 0.2 | 0.2 | 0.0 | 0.2 | 13.7 |
| Vβ5.3 | 0.1 | 0.1 | 1.6 | 0.4 | 1.9 | 0.0 | 0.0 | 0.0 | 0.0 | 0.0 | 0.3 | 0.1 |
| Vβ7.1 | 0.2 | 0.3 | 0.7 | 2.1 | 0.9 | 3.4 | 0.2 | 0.1 | 0.1 | 0.0 | 0.1 | 0.1 |
| Vβ7.2 | 6.6 | 0.0 | 0.3 | 0.1 | 0.9 | 1.7 | 0.2 | 0.1 | 0.0 | 0.0 | 1.1 | 0.1 |
| Vβ8 | 1.9 | 1.7 | 3.7 | 0.6 | 2.9 | 0.1 | 0.4 | 0.4 | 42.7 | 4.1 | 1.2 | 0.3 |
| Vβ9 | 1.6 | 0.0 | 0.8 | 0.3 | 0.3 | 0.1 | 0.1 | 0.1 | 40.5 | 0.1 | 1.7 | 0.0 |
| Vβ11 | 0.5 | 0.0 | 0.5 | 0.2 | 1.5 | 0.0 | 0.1 | 0.7 | 0.3 | 0.0 | 0.6 | 2.3 |
| Vβ12 | 3.2 | 0.9 | 1.9 | 0.9 | 2.5 | 0.1 | 0.7 | 1.8 | 0.0 | 0.0 | 0.6 | 0.3 |
| Vβ13.1 | 2.4 | 0.3 | 4.6 | 10.4 | 1.5 | 0.5 | 2.6 | 4.2 | 0.0 | 0.1 | 0.8 | 0.0 |
| Vβ13.2 | 3.1 | 0.2 | 1.4 | 0.4 | 1.8 | 0.2 | 0.2 | 0.1 | 0.0 | 0.0 | 0.2 | 0.6 |
| Vβ13.6 | 2.8 | 9.1 | 3.6 | 0.5 | 1.2 | 0.1 | 0.2 | 0.1 | 0.2 | 0.1 | 0.2 | 0.0 |
| Vβ14 | 1.9 | 1.4 | 2.0 | 3.6 | 1.3 | 63.3 | 0.4 | 0.7 | 0.0 | 0.0 | 1.0 | 5.3 |
| Vβ16 | 2.4 | 1.0 | 6.9 | 6.9 | 3.3 | 0.3 | 4.1 | 1.1 | 0.0 | 0.1 | 0.8 | 0.0 |
| Vβ17 | 4.6 | 0.1 | 2.9 | 1.8 | 0.8 | 0.0 | 0.2 | 0.0 | 0.0 | 0.0 | 1.5 | 0.0 |
| Vβ18 | 2.4 | 0.1 | 0.2 | 0.0 | 0.5 | 0.0 | 0.1 | 0.0 | 0.4 | 0.0 | 0.4 | 0.2 |
| Vβ20 | 7.0 | 5.9 | 6.6 | 13.8 | 4.6 | 0.1 | 3.7 | 0.1 | 0.0 | 0.4 | 0.7 | 0.2 |
| Vβ21.3 | 0.4 | 0.5 | 3.8 | 4.3 | 0.4 | 0.1 | 0.1 | 0.1 | 0.0 | 3.2 | 6.2 | 0.1 |
| Vβ22 | 11.1 | 0.2 | 3.3 | 1.7 | 2.8 | 0.2 | 6.2 | 1.8 | 0.1 | 0.0 | 15.1 | 2.1 |
| Vβ23 | 3.7 | 0.3 | 0.4 | 0.1 | 0.8 | 0.8 | 0.9 | 0.5 | 1.6 | 0.5 | 0.1 | 4.6 |

| | Panc 7 | | Panc 8 | | Panc 9 | | Panc 10 | | Panc 11 | |
|---|---|---|---|---|---|---|---|---|---|---|
| | CD4 | CD8 | CD4 | CD4 | CD8 | CD4 | CD8 | CD8 | CD4 | CD8 |
| Vβ1 | 0.3 | 0.1 | 1.5 | 4.4 | 7.1 | 13.0 | 0.5 | 1.2 | 0.6 | 0.1 |
| Vβ2 | 0.1 | 0.7 | 7.4 | 4.1 | 39.8 | 2.4 | 0.2 | 1.7 | 12.0 | 0.0 |
| Vβ3 | 5.1 | 28.7 | 2.4 | 3.7 | 1.3 | 0.0 | 0.0 | 12.0 | 1.0 | 0.0 |
| Vβ4 | 0.1 | 0.0 | 0.1 | 6.0 | 0.1 | 0.0 | 0.0 | 0.0 | 3.2 | 0.0 |
| Vβ5.1 | 41.1 | 6.2 | 9.3 | 10.8 | 1.1 | 0.6 | 0.7 | 0.0 | 3.6 | 0.1 |
| Vβ5.2 | 2.4 | 0.2 | 0.3 | 1.3 | 9.4 | 0.1 | 0.1 | 0.2 | 0.1 | 0.1 |
| Vβ5.3 | 0.0 | 0.9 | 0.6 | 0.7 | 18.0 | 0.2 | 0.0 | 0.1 | 0.0 | 0.0 |
| Vβ7.1 | 0.1 | 0.2 | 5.1 | 0.5 | 1.5 | 0.0 | 0.0 | 0.3 | 0.6 | 0.0 |
| Vβ7.2 | 1.0 | 0.4 | 5.3 | 1.6 | 0.1 | 0.0 | 0.0 | 3.8 | 13.9 | 0.0 |
| Vβ8 | 5.4 | 0.5 | 2.3 | 2.7 | 1.4 | 0.4 | 0.3 | 2.2 | 0.5 | 0.1 |
| Vβ9 | 0.2 | 0.1 | 1.0 | 3.4 | 1.2 | 0.5 | 0.2 | 0.1 | 4.8 | 0.0 |
| Vβ11 | 4.0 | 1.0 | 0.2 | 0.6 | 0.3 | 0.1 | 0.2 | 0.0 | 1.0 | 0.1 |
| Vβ12 | 0.0 | 0.0 | 0.1 | 1.6 | 0.5 | 0.0 | 0.0 | 0.1 | 0.4 | 0.0 |
| Vβ13.1 | 8.1 | 10.0 | 5.6 | 3.7 | 0.1 | 35.5 | 0.1 | 16.5 | 5.0 | 0.0 |
| Vβ13.2 | 0.3 | 1.1 | 0.3 | 6.2 | 0.7 | 0.2 | 0.0 | 0.0 | 0.5 | 99.2 |
| Vβ13.6 | 1.7 | 0.1 | 3.2 | 2.0 | 0.3 | 0.6 | 0.0 | 6.9 | 0.4 | 0.0 |
| Vβ14 | 3.2 | 2.8 | 0.6 | 3.6 | 0.8 | 0.1 | 0.0 | 1.4 | 0.4 | 0.1 |
| Vβ16 | 0.1 | 0.3 | 0.1 | 1.0 | 0.2 | 0.0 | 0.2 | 25.4 | 0.5 | 0.0 |
| Vβ17 | 1.0 | 32.9 | 1.3 | 7.5 | 0.3 | 0.1 | 12.3 | 0.1 | 12.4 | 0.0 |
| Vβ18 | 1.0 | 0.2 | 3.6 | 0.5 | 0.2 | 0.1 | 0.2 | 0.0 | 0.4 | 0.0 |
| Vβ20 | 0.1 | 0.0 | 1.4 | 2.5 | 0.5 | 0.1 | 0.1 | 0.4 | 0.1 | 0.0 |
| Vβ21.3 | 2.4 | 0.0 | 0.6 | 2.4 | 0.1 | 0.9 | 0.0 | 0.3 | 0.5 | 0.0 |
| Vβ22 | 0.2 | 2.6 | 1.4 | 3.4 | 1.0 | 3.6 | 0.1 | 0.6 | 1.9 | 0.7 |
| Vβ23 | 0.1 | 0.1 | 0.2 | 4.5 | 0.7 | 0.6 | 0.1 | 6.7 | 0.1 | 0.0 |
| | Panc 12 | | Panc 13 | | Panc 14 | | Panc 15 | | Panc 16 | |
| | CD4 | CD8 | CD4 | CD8 | CD4 | CD8 | CD4 | CD8 | CD4 | CD8 |
| Vβ1 | 1.4 | 25.8 | 0.1 | 0.3 | 0.9 | 16.2 | 0.6 | 2.8 | 0.3 | 0.4 |
| Vβ2 | 7.5 | 4.8 | 1.5 | 0.6 | 5.1 | 0.7 | 29.3 | 1.5 | 48.9 | 1.0 |
| Vβ3 | 1.7 | 8.8 | 0.0 | 0.0 | 13.3 | 2.0 | 1.1 | 17.9 | 11.7 | 0.2 |
| Vβ4 | 2.2 | 0.4 | 4.8 | 0.0 | 0.9 | 0.1 | 8.3 | 1.2 | 0.1 | 0.1 |
| Vβ5.1 | 3.4 | 0.5 | 4.6 | 0.2 | 2.7 | 0.8 | 4.2 | 1.6 | 15.2 | 0.2 |
| Vβ5.2 | 0.3 | 1.8 | 0.1 | 0.1 | 0.4 | 0.1 | 2.3 | 0.8 | 1.2 | 0.1 |
| Vβ5.3 | 0.3 | 1.6 | 0.1 | 0.0 | 0.1 | 0.3 | 4.2 | 0.3 | 0.9 | 0.0 |
| Vβ7.1 | 1.6 | 2.8 | 0.1 | 0.3 | 0.3 | 6.3 | 0.9 | 1.8 | 0.3 | 2.2 |
| Vβ7.2 | 0.4 | 0.1 | 0.0 | 0.0 | 0.7 | 4.8 | 0.4 | 0.1 | 0.0 | 0.0 |
| Vβ8 | 3.6 | 1.8 | 1.1 | 0.5 | 1.7 | 0.8 | 1.9 | 4.1 | 0.5 | 0.3 |
| Vβ9 | 6.7 | 1.0 | 2.4 | 0.1 | 9.7 | 2.3 | 2.0 | 2.0 | 1.8 | 0.4 |
| Vβ11 | 0.8 | 0.3 | 0.2 | 0.2 | 0.3 | 0.2 | 1.4 | 0.7 | 0.4 | 0.2 |
| Vβ12 | 0.3 | 1.0 | 0.3 | 0.0 | 1.5 | 5.9 | 0.3 | 0.2 | 0.3 | 0.1 |
| Vβ13.1 | 9.2 | 0.3 | 0.5 | 0.2 | 4.3 | 19.6 | 0.7 | 0.8 | 2.6 | 1.1 |
| Vβ13.2 | 2.5 | 1.4 | 0.2 | 0.2 | 4.4 | 0.9 | 1.8 | 23.0 | 0.4 | 0.6 |
| Vβ13.6 | 1.5 | 0.2 | 0.5 | 0.2 | 0.3 | 1.9 | 0.6 | 0.9 | 0.3 | 0.4 |
| Vβ14 | 0.7 | 3.5 | 0.1 | 0.3 | 2.9 | 2.5 | 0.9 | 7.2 | 2.1 | 0.6 |
| Vβ16 | 0.1 | 0.1 | 0.0 | 0.2 | 0.2 | 0.8 | 0.2 | 0.2 | 0.1 | 0.0 |
| Vβ17 | 3.3 | 2.3 | 31.1 | 0.9 | 9.9 | 1.4 | 1.3 | 1.9 | 1.5 | 0.5 |
| Vβ18 | 0.3 | 0.3 | 0.5 | 0.1 | 0.9 | 3.1 | 3.3 | 3.6 | 0.4 | 3.3 |
| Vβ20 | 1.4 | 0.2 | 0.0 | 0.0 | 0.7 | 0.3 | 0.5 | 0.6 | 0.0 | 0.8 |
| Vβ21.3 | 0.5 | 8.0 | 0.5 | 0.0 | 1.2 | 0.5 | 0.4 | 0.4 | 0.2 | 1.6 |
| Vβ22 | 5.2 | 2.0 | 26.0 | 65.9 | 3.6 | 0.8 | 0.7 | 2.9 | 6.2 | 6.4 |
| Vβ23 | 0.2 | 0.4 | 0.3 | 1.0 | 0.5 | 0.7 | 1.8 | 7.4 | 0.3 | 0.3 |

IFNγ production is measured after 3 days after exposure of TIL to autologous tumor cells (single cell suspension). High level of IFNγ production is seen after with autologous tumor cells only (see FIG. 35) IFNγ production is complete blocked with the antibody against W6/32 (blocking CD8+ TIL). As a control, the antibody L243 blocking CD4+ TIL is not able to block reactivity, the reactive T-cells in expanded cell lymphocytes are all CD8+. The data show that the cytokine cocktail expands TIL that are very focused and specifically recognized autologous tumor cells from patients with pancreatic cancer. Monoclonal TCR families in TIL from patients with pancreatic cancer: IL-2, IL-15 and 11-21 expanded preferentially individual TCR VB families, such a preferential expansion of T-cell families is associated with antigen-specificy and focused anti-tumor reactivity. Some of these preferentially expanded VB families contain monoclonal T-cells, defined by a single TCR VB chain. Such monoclonal TCR expansion is indicative a focused, anti-cancer response. The functional response analyses showed that monoclonally expanded TIL recognize autologous tumor cells

### Example 39

### Analysis of the Cytolytic Response of Expanded TIL from Patients with Glioblastoma Against Autologous Tumor Cells

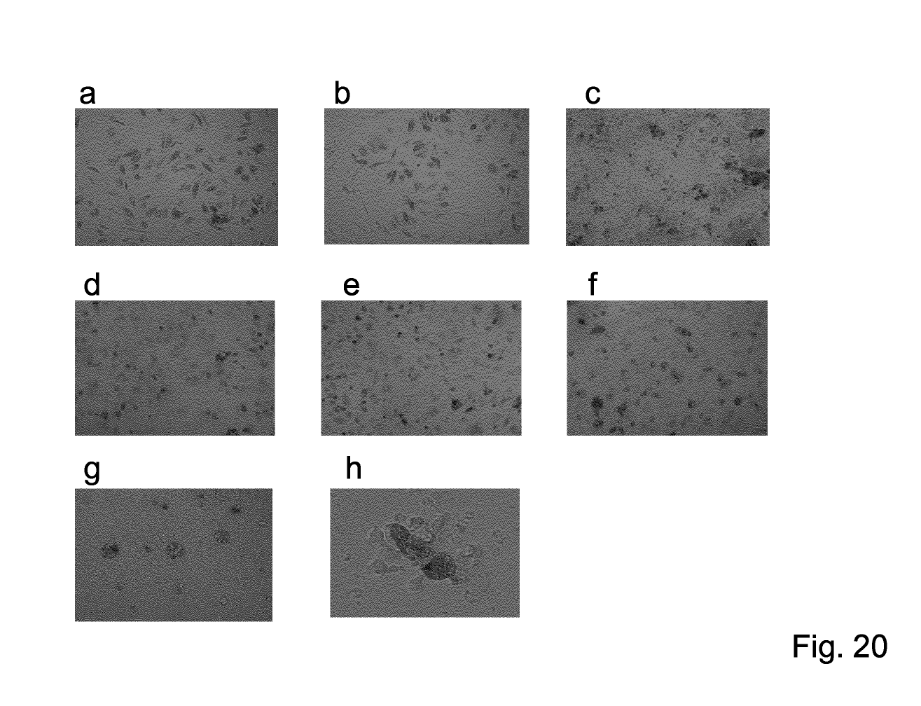

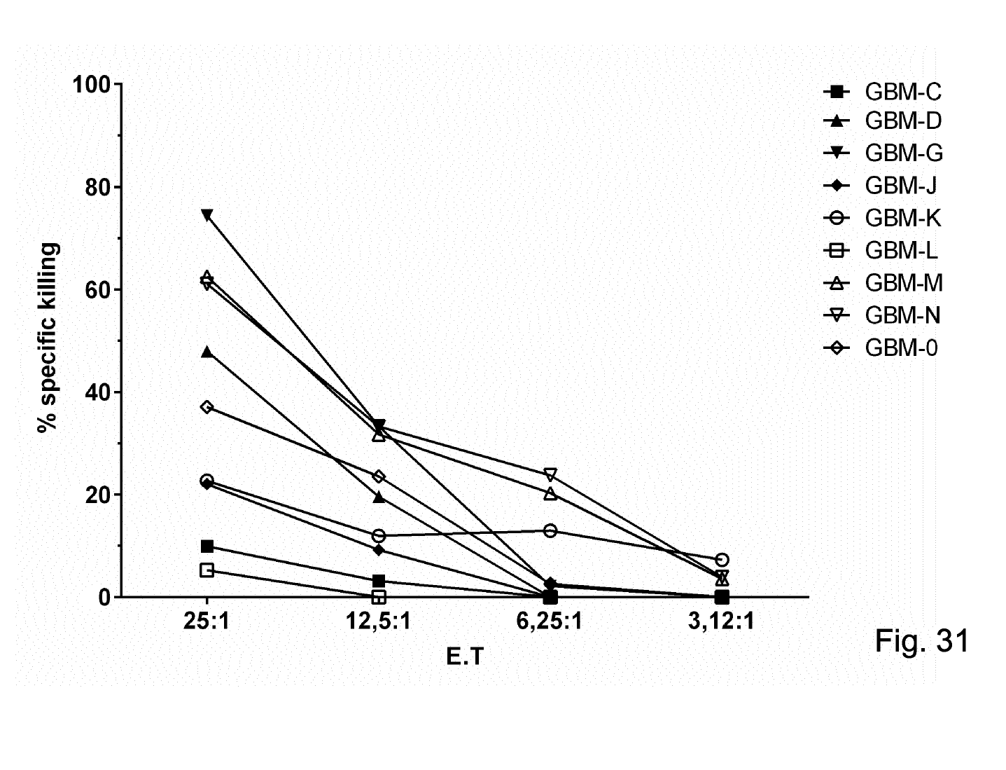

Tumor tissue was obtained from glioblastoma patients. TILs were expanded from the tumor tissue with the cytokine cocktail of IL-2, IL-15 and IL-21 according to the protocol of Example 28. Autologous tumor cells were generated according to the protocol in Example 29. Autologous tumor cells are labeled with radioactivity and cultured with expanded TIL for 4 h. Killing of tumor cells leads to release of radioactivity which is then measured. The principle of the method is shown in FIG. 21. The results are shown in FIG. 31 confirming a cytotoxic effect dependent on the ratio of TILs to tumor cells. The data show that the cytokine cocktail expands TIL that are strongly cytoxic against autologous tumor cells.

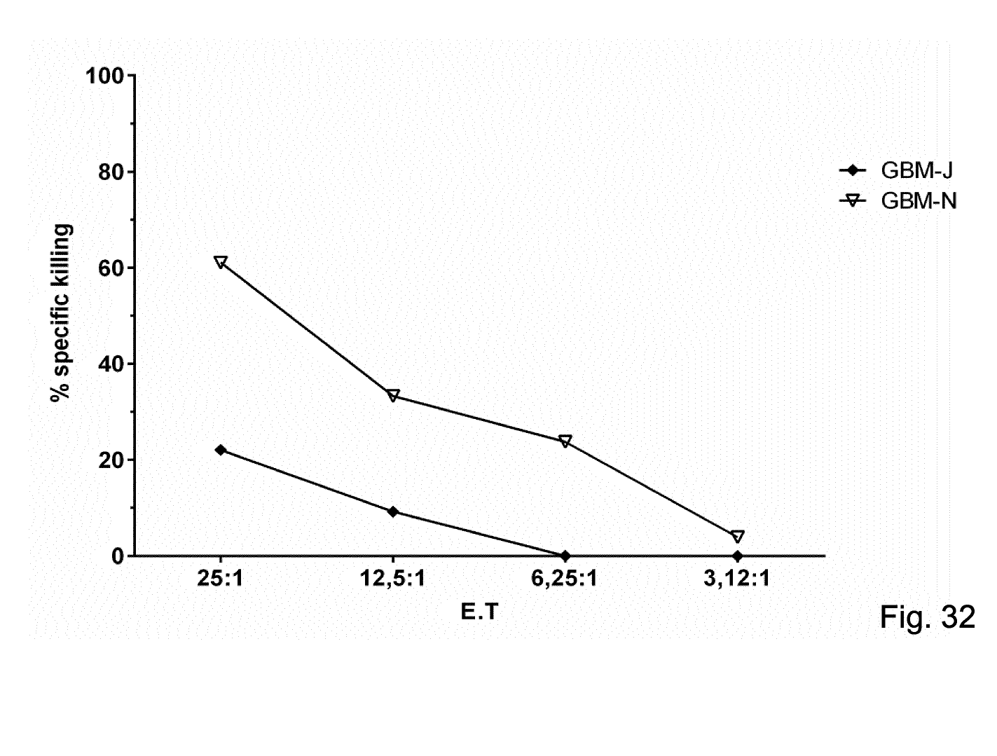

Alternatively, expanded monoclonal T-cells and/or preferentially expanded TIL were tested for their cytotoxicity with the same test. The results are shown in FIG. 32. Table 16 shows that these immune responses are specifically directed against the autologous tumor cells. The data shows that the cytokine cocktail expands TIL with a specific reactivity, including cytotoxic responses, against autologous tumor.

B)

In a parallel experiment the autologous tumor cells were incubated for three days with the TIL expanded from glioblastoma with the cytokine cocktail of IL-15, IL-21 and IL-2. After incubation IFNγ production (pg/mL) was measured by ELISA. In order to identify whether CD4+ or CD8+ cells are responsible for the tumor responses antibodies blocking either MHC class I antigen responses (W6/32) affecting CD8+ T-cells, or blocking HLA-DR (MHC class II responses) affecting CD4+ T-cells were used. The results are summarized in Table 16.

TABLE 16

| | | autologous | autologous | autologous |
|---|---|---|---|---|
| Diagnosis | Label | Tumor | Tumor + W6/32 | Tumor + L243 |
| GBM (grade IV) | GBM-A | 15, 40 | 0, 00 | 0, 00 |
| PXA (grade II-III) | GBM-B | 251, 19 | 109, 75 | 0, 00 |
| GBM (grade IV) | GBM-C | 13, 32 | 30, 94 | 0, 00 |
| GBM (grade IV) | GBM-D | 1123, 76 | 752, 54 | 65, 65 |
| GBM (grade IV) | GBM-E | 424, 86 | 114, 93 | 23, 28 |
| GBM (grade IV) | GBM-F | 678, 36 | 251, 12 | 0, 00 |
| GBM (grade IV) | GBM-G | 0, 00 | 0, 00 | 0, 00 |
| GBM (grade IV) | GBM-H | 141, 89 | 131, 31 | 51, 47 |
| GBM (grade IV) | GBM-I | 971, 12 | 356, 22 | 176, 89 |
| GBM (grade IV) | GBM-J | 100, 75 | 89, 32 | 19, 56 |
| GBM (grade IV) | GBM-K | 89, 47 | 0, 00 | 0, 00 |
| AO (grade III) | GBM-L | 0, 00 | 0, 00 | 0, 00 |
| GBM (grade IV) | GBM-M | 0, 00 | 0, 00 | 0, 00 |
| O (grade II) | GBM-N | 0, 00 | 0, 00 | 0, 00 |
| AE (grade III) | GBM-O | 0, 00 | 0, 00 | 0, 00 |
| Relapse Necrosis | GBM-P | 36, 26 | 0, 21 | 0, 00 |

The TIL that showed absent IFNγ production were strongly cytotoxic, as measured in a standard CR51 release assay. The data show that TIL produce IFNgamma against autologous tumor cells in a specific fashion.

### Example 40

### Analysis of the Cytolytic Response of Expanded TIL from Patients with Pancreas Cancer Against Autologous Tumor Cells

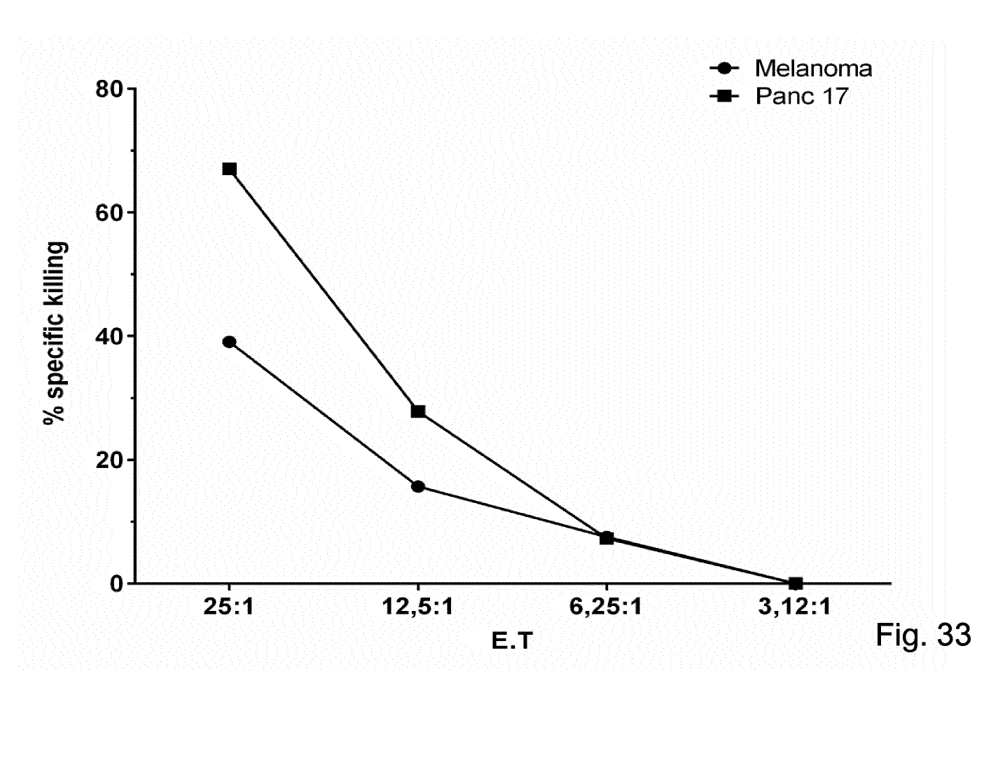

Tumor tissue was obtained from pancreatic cancer patients. TILs were expanded from the tumor tissue with the cytokine cocktail of IL-2, IL-15 and IL-21 according to the protocol of Example 28. Autologous tumor cells were generated according to the protocol in Example 29. Autologous tumor cells are labeled with radioactivity and cultured with expanded TIL for 4 h. Killing of tumor cells leads to release of radioactivity which is then measured. The principle of the method is shown in FIG. 7. For comparison and as a control, an autologous (melanoma)-TIL system was used. The results are shown in FIG. 33 confirming a cytotoxic effect. The data show that the cytokine cocktail expands TIL also from patients with pancreatic cancer. These TIL are highly focused—based on the TCR usage—and show a specific reactivity, including cytotoxic responses, against autologous tumor.

### Example 41

### Analysis of the CXCR3 Expression CD4+ Cell Distribution

Tumor tissue was obtained from glioblastoma patients. TILs were expanded from the tumor tissue with the cytokine cocktail of IL-2, IL-15 and IL-21 according to the protocol of Example 27. Cells were analyzed by flow cytometry for marker expression that defined T-cell function and homing.

The results of the analysis are summarized in Table 17. The Th1 cells as well as the CXCR3 were less than 10% prior to expansion. The data show that the cytokine cocktail leads to a T-cell product in which the CD8+ cells consists essentially of CXCR3+ CD8+ T-cells. This phenotype is enabled to enter into the tumor tissue. The CD4+ T-cells almost all have a T_{H1} profile. The T_{H1} profile (IFNgamma and TNFalpha production) leads to improved anti-tumor responses.

Also CD3+CD4-CD8-(DN) T-cells are present, a T-cell subset, associated with strong-autoimmune and tumor responses, which also expresses the marker CXCR3 that enables better penetration into tumor tissue.

TABLE 17

| Lymphocytes | | | 76, 3 |
|---|---|---|---|
| CD3+ | | | 84, 3 |
| CD8+ | CD8+ | | 25 |
| | c-kit | | 0, 49 |
| | CD107a+ | | 1, 58 |
| | CXCR3+ | | 81, 1 |
| DP | DP | | 2, 55 |
| CD4+ | CD4+ | | 68, 9 |
| | c-kit | | 0, 073 |
| | CCR6+ | CCR6+ | 36, 2 |
| | | TH1* | 89, 3 |
| | | TH17 | 0, 047 |
| | CCR6− | CCR6− | 63, 8 |
| | | TH1 | 91, 5 |
| | | TH2 | 0, 038 |

| | | |
|---|---|---|
| | CD107a+ | 1 |
| DN | DN | 3, 56 |
| | c-kit | 1, 36 |
| | CD107a+ | 1 |
| | CXCR3+ | 59, 1 |

### Example 42

### Recognition of Commonly Shared Tumor Antigens by TIL from Patients with Pancreatic Cancer

TAAs as 15 overlapping peptides were incubated for three days with TIL and IFNγ production (pg/mL) was measured by ELISA. In addition, antigen responses were either blocked by an antibody blocking MHC class I antigen responses (W6/32) affecting CD8+ T-cells, or blocking HLA-DR (MHC class II responses, L243) affecting CD4+ T-cells. The IFNγ production (pg/mL) in each of the samples is shown in Table 18. IFNγ production in TIL that is antigen-specific, as shown by blocking with anti-MHC class I (blocking CD8+ T-cells) or with anti-MHC class II (blocking CD4+) T-cells. The results confirm that TIL produce IFNγ against commonly shared cancer antigens, particularly NY-ESO-1 or mesothelin from patients with pancreatic cancer.

TABLE 18

| | | | NY- | | | | | | |
|---|---|---|---|---|---|---|---|---|---|
| | NY- | NY-ESO- | ESO-1 + | | Survivin + | Survivin + | | Mesothelin + | Mesothelin + |
| | ESO-1 | 1 + W6/32 | L243 | Survivin | W6/32 | L243 | Mesothelin | W6/32 | L243 |
| Panc 1 | 6.97 | 0 | 0 | 0 | 0 | 0 | 262.23 | 15.26 | 0 |
| Panc 2 | 24.20 | 0 | 0 | 0 | 0 | 0 | 291.27 | 13.50 | 0 |
| Panc 6 | 51.02 | 31.96 | 33.74 | 77.99 | 49.24 | 36.72 | 44.46 | 34.34 | 27.21 |
| Panc 10 | 4.13 | 0 | 0 | 66.06 | 0 | 0 | 78.70 | 72.88 | 0 |
| Panc 11 | 0 | 0 | 0 | 0 | 0 | 0 | 131.16 | 0 | 0 |
| Panc 14 | 10.47 | 0 | 0 | 0 | 0 | 0 | 275.15 | n.d. | 249.95 |
| Panc 16 | 173.19 | 0 | 0 | 8.35 | 0 | 0 | 19.64 | 0 | n.d. |

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCES

1. Dunn G P, Old L J, Schreiber R D. The immunobiology of cancer immunosurveillance and immunoediting. Immunity. 2004;21(2):137-48.
2. Rosenberg S A, Restifo N P, Yang J C, Morgan R A, Dudley M E. Adoptive cell transfer: a clinical path to effective cancer immunotherapy. Nature reviews Cancer. 2008;8(4):299-308.
3. Rosenberg S A, Packard B S, Aebersold P M, Solomon D, Topalian S L, Toy S T, et al. Use of tumor-infiltrating lymphocytes and interleukin-2 in the immunotherapy of patients with metastatic melanoma. A preliminary report. The New England journal of medicine. 1988;319(25):1676-80.
4. Robbins P F, Lu Y C, EI-Gamil M, Li Y F, Gross C, Gartner J, et al. Mining exomic sequencing data to identify mutated antigens recognized by adoptively transferred tumor-reactive T cells. Nat Med. 2013;19(6):747-52.
5. Tran E, Turcotte S, Gros A, Robbins P F, Lu Y C, Dudley M E, et al. Cancer immunotherapy based on mutation-specific CD4+ T cells in a patient with epithelial cancer. Science. 2014;344(6184):641-5.
6. Li Y, Liu S, Hernandez J, Vence L, Hwu P, Radvanyi L. MART-1-specific melanoma tumor-infiltrating lymphocytes maintaining CD28 expression have improved survival and expansion capability following antigenic restimulation in vitro. Journal of immunology. 2010;184(1):452-65.
7. June C H. Principles of adoptive T cell cancer therapy. The Journal of clinical investigation. 2007;117(5)1204-12.
8. Dudley M E, Wunderlich J, Nishimura M I, Yu D, Yang J C, Topalian S L, et al. Adoptive transfer of cloned melanoma-reactive T lymphocytes for the treatment of patients with metastatic melanoma. Journal of immunotherapy. 2001;24(4):363-73.
9. Dudley M E, Wunderlich J R, Shelton T E, Even J, Rosenberg S A. Generation of tumor-infiltrating lymphocyte cultures for use in adoptive transfer therapy for melanoma patients. Journal of immunotherapy. 2003;26(4):332-42.
10. Weber J, Atkins M, Hwu P, Radvanyi L, Sznol M, Yee C, et al. White paper on adoptive cell therapy for cancer with tumor-infiltrating lymphocytes: a report of the CTEP subcommittee on adoptive cell therapy. Clinical cancer research: an official journal of the American Association for Cancer Research. 2011;17(7):1664-73.
11. Tran K Q, Zhou J, Durflinger K H, Langhan M M, Shelton T E, Wunderlich J R, et al. Minimally cultured tumor-infiltrating lymphocytes display optimal characteristics for adoptive cell therapy. Journal of immunotherapy. 2008;31(8):742-51.
12. Nguyen L T, Yen P H, Nie J, Liadis N, Ghazarian D, Al-Habeeb A, et al. Expansion and characterization of human melanoma tumor-infiltrating lymphocytes (TILs). PloS one. 2010;5(11):e13940.
13. Zhou J, Dudley M E, Rosenberg S A, Robbins P F. Selective growth, in vitro and in vivo, of individual T cell clones from tumor-infiltrating lymphocytes obtained from patients with melanoma. Journal of immunology. 2004;173(12):7622-9.
14. Hunder N N, Wallen H, Cao J, Hendricks D W, Reilly J Z, Rodmyre R, et al.Treatment of metastatic melanoma with autologous CD4+ T cells against NY-ESO-1. The New England journal of medicine. 2008;358(25):2698-703.
15. Kagamu H, Shu S. Purification of L-selectin(low) cells promotes the generation of highly potent CD4 antitumor effector T lymphocytes. Journal of immunology. 1998;160(7):3444-52.
16. Xie Y, Akpinarli A, Mans C, Hipkiss E L, Lane M, Kwon E K, et al. Naive tumorspecific CD4(+) T cells differentiated in vivo eradicate established melanoma. The Journal of experimental medicine. 2010;207(3):651-67.
17. Mackensen A, Meidenbauer N, Vogl S, Laumer M, Berger J, Andreesen R. Phase I study of adoptive T-cell therapy using antigen-specific CD8+ T cells for the treatment of patients with metastatic melanoma. Journal of clinical oncology: official journal of the American Society of Clinical Oncology. 2006;24(31):5060-9.
18.Jager E, Gnjatic S, Nagata Y, Stockert E, Jager D, Karbach J, et al. Induction of primary NY-ESO-1 immunity: CD8+ T lymphocyte and antibody responses in peptidevaccinated patients with NY-ESO-1+ cancers. Proc Natl Acad Sci U S A. 2000;97(22):12198-203.
19.Ho W Y, Nguyen H N, Wolfl M, Kuball J, Greenberg P D. In vitro methods for generating CD8+ T-cell clones for immunotherapy from the naive repertoire. Journal of immunological methods. 2006;310(1-2): 40-52.
20. Dudley M E, Wunderlich J R, Robbins P F, Yang J C, Hwu P, Schwartzentruber D J, et al. Cancer regression and autoimmunity in patients after clonal repopulation with antitumor lymphocytes. Science. 2002;298(5594):850-4.
21. Lord G M, Matarese G, Howard J K, Baker R J, Bloom S R, Lechler R I. Leptin modulates the T-cell immune response and reverses starvation-induced immunosuppression. Nature. 1998;394(6696):897-901.
22. Tanaka M, Suganami T, Kim-Saijo M, Toda C, Tsuiji M, Ochi K, et al. Role of central leptin signaling in the starvation-induced alteration of B-cell development. The Journal of neuroscience : the official journal of the Society for Neuroscience. 2011;31(23):8373-80.
23. Ravindran R, Khan N, Nakaya H I, Li S, Loebbermann J, Maddur M S, et al. Vaccine activation of the nutrient sensor GCN2 in dendritic cells enhances antigen presentation. Science. 2014;343(6168):313-7.
24. Pearson C, Silva A, Seddon B. Exogenous amino acids are essential for interleukin-7 induced CD8 T cell growth. [corrected]. PloS one. 2012;7(4):e33998.
25. Schwartzentruber D J, Hom S S, Dadmarz R, White D E, Yannelli J R, Steinberg S M, et al. In vitro predictors of therapeutic response in melanoma patients receiving tumor-infiltrating lymphocytes and interleukin-2. Journal of clinical oncology : official journal of the American Society of Clinical Oncology. 1994;12(7):1475-83.
26. Pittet M J, Speiser D E, Valmori D, Cerottini J C, Romero P. Cutting edge: cytolytic effector function in human circulating CD8+ T cells closely correlates with CD56 surface expression. Journal of immunology. 2000;164(3)1148-52.
27. Shen X, Zhou J, Hathcock K S, Robbins P, Powell D J, Jr., Rosenberg S A, et al.Persistence of tumor infiltrating lymphocytes in adoptive immunotherapy correlates with telomere length. Journal of immunotherapy. 2007;30(1)123-9.
28. Zhou J, Shen X, Huang J, Hodes R J, Rosenberg S A, Robbins P F. Telomere length of transferred lymphocytes correlates with in vivo persistence and tumor regression in melanoma patients receiving cell transfer therapy. Journal of immunology. 2005;175(10):7046-52.
29. Inozume T, Hanada K, Wang Q J, Ahmadzadeh M, Wunderlich J R, Rosenberg S A, et al. Selection of CD8+PD-1+ lymphocytes in fresh human melanomas enriches for tumor-reactive T cells. Journal of immunotherapy. 2010;33(9):956-64.
30. Zeng R, Spolski R, Finkelstein S E, Oh S, Kovanen P E, Hinrichs C S, et al. Synergy of IL-21 and IL-15 in regulating CD8+ T cell expansion and function. The Journal of experimental medicine. 2005;201(1):139-48.
31. Liu D, Song L, Wei J, Courtney A N, Gao X, Marinova E, et al. IL-15 protects NKT cells from inhibition by tumor-associated macrophages and enhances antimetastatic activity. The Journal of clinical investigation. 2012;122(6):2221-33.
32. Li Y, Bleakley M, Yee C. IL-21 influences the frequency, phenotype, and affinity of the antigen-specific CD8 T cell response. Journal of immunology. 2005;175(4): 2261-9.
33. Gattinoni L, Ji Y, Restifo N P. Wnt/beta-catenin signaling in T-cell immunity and cancer immunotherapy. Clinical cancer research: an official journal of the American Association for Cancer Research. 2010;16(19):4695-701.
34. Gattinoni L, Zhong X S, Palmer D C, Ji Y, Hinrichs C S, Yu Z, et al. Wnt signaling arrests effector T cell differentiation and generates CD8+ memory stem cells. Nat Med. 2009;15(7):808-13.
35. Yi J S, Du M, Zajac A J. A vital role for interleukin-21 in the control of a chronic viral infection. Science. 2009;324(5934):1572-6.
36. Jager D, Karbach J, Pauligk C, Seil I, Frei C, Chen Y T, et al. Humoral and cellular immune responses against the breast cancer antigen NY-BR-1: definition of two HLAA2 restricted peptide epitopes. Cancer immunity. 2005;5:11.

37. Jager E, Karbach J, Gnjatic S, Jager D, Maeurer M, Atmaca A, et al. Identification of a naturally processed NY-ESO-1 peptide recognized by CD8+ T cells in the context of HLA-B51. Cancer immunity. 2002;2:12.
38. Di Tomaso T, Mazzoleni S, Wang E, Sovena G, Clavenna D, Franzin A, et al.lmmunobiological characterization of cancer stem cells isolated from glioblastoma patients. Clinical cancer research: an official journal of the American Association for Cancer Research. 2010;16(3):800-13.
39. Natsume A, Wakabayashi T, Tsujimura K, Shimato S, Ito M, Kuzushima K, et al. The DNA demethylating agent 5-aza-2′-deoxycytidine activates NY-ESO-1 antigenicity in orthotopic human glioma. International journal of cancer Journal international du cancer. 2008;122(11):2542-53.
40. Konkankit W, Kim W, Koya RC, Eskin A, Dam MA, Nelson S, et al. Decitabine immunosensitizes human gliomas to NY-ESO-1 specific T lymphocyte targeting through the Fas/Fas ligand pathway. Journal of translational medicine. 2011;9:192.
41. Maeurer M, Hohn H, Castelli C, Salter R D, Necker A, Reichert T, et al. Antigen recognition by T cells: a strong sense of structure. Trends in immunology. 2001;22(11):599-601.
42. Maeurer M J, Gollin S M, Martin D, Swaney W, Bryant J, Castelli C, et al. Tumor escape from immune recognition: lethal recurrent melanoma in a patient associated with downregulation of the peptide transporter protein TAP-1 and loss of expression of the immunodominant MART-1/Melan-A antigen. The Journal of clinical investigation. 1996;98(7):1633-41.
43. Maeurer M J, Necker A, Salter R D, Castelli C, Hohn H, Karbach J, et al. Improved detection of melanoma antigen-specific T cells expressing low or high levels of CD8 by HLA-A2 tetramers presenting a Melan-A/Mart-1 peptide analogue. International journal of cancer Journal international du cancer. 2002;97(1):64-71.
44. Magalhaes I, Vudattu N K, Ahmed R K, Kuhlmann-Berenzon S, Ngo Y, Sizemore D R, et al. High content cellular immune profiling reveals differences between rhesus monkeys and men. Immunology. 2010;131(1)128-40.

1. Composition for expanding lymphocytes comprising at least two types of cytokines selected from interleukin 2 (IL-2), interleukin 15 (IL-15) and interleukin 21 (IL-21).

2. Composition according to claim 1, comprising two or three types of cytokines.

3. Composition according to claim 1 or 2, wherein the composition is in liquid form, in particular a cell culture medium.

4. Composition according to claim 3, wherein the concentration of IL-2 in the liquid composition is in the range from 10 to 6000 U/ml, preferably in the range from 500 to 2000 U/ml, more preferably in the range from 800 to 1200 U/mI.

5. Composition according to claim 3 or 4, wherein the concentration of IL-15 is in the range from 0.1 to 100 ng/ml, preferably in the range from 2 to 50 ng/ml, more preferably in the range from 5 to 20 ng/ml.

6. Composition according to any of claims 3 to 5, wherein the concentration of IL-21 is in the range from 0.1 to 100 ng/ml, preferably in the range from 2 to 50 ng/ml, more preferably in the range from 5 to 20 ng/ml.

7. Method of preparing a population of clinically relevant lymphocytes, comprising the steps of:
obtaining a body sample from a mammal in particular a tissue sample or body liquid sample, comprising at least one lymphocyte and optionally separating the cells in the body sample,
culturing the body sample in-vitro to expand and/or stimulate lymphocytes in the sample wherein the culturing comprises using IL-2, IL-15 and/or IL-21,
and optionally determining the presence of clinically relevant lymphocyte in the cultured sample.

8. Method according to claim 7, wherein the clinically relevant lymphocytes are selected from tumor-reactive lymphocytes, pathogen reactive lymphocytes and autoimmune reactive lymphocytes, preferably tumor-reactive lymphocytes.

9. Method according to claim 7 or 8, wherein the body sample is selected from the group consisting of peripheral blood, cord blood, bone marrow, lymph nodes liver, pleural effusion, thorax, abdominal cavity, synvial fluid, peritoneum, retroperitoneal space, thymus, and tumor.

10. Method according to claim 9, wherein the body sample is selected from peripheral blood.

11. Method according to claim any of claims 7 to 10, wherein the mammal, in particular a human, is selected from a mammal with a tumor disease, a mammal at risk of developing a tumor disease, a mammal with an infectious disease, a mammal at risk of developing an infectious disease, a mammal with an auto immune disease, a mammal at risk of developing an autoimmune disease.

12. Method according to any of claims 7 to 11, wherein the in-vitro culturing comprises a first expansion step comprising an incubation in culture medium comprising IL-2, IL-15 and IL-21 until lymphocytes become detectable.

13. Method according to claim 12, wherein the time of incubation of the first expansion step is in the range from 6 hours to 180 days preferably in the range from 4 to 10, more preferably in the range from 6 to 8 days, most preferably about 7 days.

14. Method according to claim 12 or 13, wherein the in-vitro culturing comprises a second expansion step comprising an incubation in culture medium comprising feeder cells and/or an antibody against CD3 in addition to IL-2, IL-15 and IL-21.

15. Method according to claim 14, wherein the ratio of feeder cells to lymphocytes is in the range from 1:1 to 1:100, preferably in the range from 1:2 to 1:50, more preferably in the range from 1:5 to 1:20, most preferably about 1:10.

16. Method according to any of claims 12 to 15, wherein the first expansion step comprises adding IL-2, IL-15 and IL-21 at the same time point to the cell culture.

17. Method according to any of claims 12 to 15, wherein the first expansion step comprises adding at least one of IL-2, IL-15 and IL-21 separately at a different time point to the cell culture.

18. Method according to claim 17, wherein first IL-21 is added and in particular IL-15 is added second before adding IL-2.

19. Method according to claim 17, wherein first IL-15 is added and in particular IL-21 is added second before adding IL-2.

20. Method according to any of claims 7 to 19, wherein the clinically relevant lymphocyte population is either one of monoclonal, oligoclonal or polyclonal.

21. Method according to any of claims 12 to 20, wherein the culture medium of the first and/or second expansion step comprises least one expansion antigen.

22. Method according to claim 21, wherein expansion antigen is a fragment of TAA, in particular a peptide comprising at least eight continuous amino acids of an amino acid sequence that is at least 80% identical to the amino acid sequences SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14

23. Method according to any of claims 12 to 22, wherein the in-vitro culturing further comprises a refocusing step, comprising an incubation in culture medium comprising refocusing cells.

24. Method according to claim 23, wherein the time of the refocusing step is in the range from 1 to 6 days, preferably 1 to 3 days.

25. Method according to claim 23 or 24, wherein the ratio of refocusing cells to lymphocytes is in the range from 1:1 to 1:100, preferably in the range from 1:5 to 1:10.

26. Method according to any of claims 7 to 25, wherein the culturing comprises the addition of a promoter compound to promote the expansion of a specific subgroup of lymphocytes, in particular gamma-delta T-cells (γδ-T-cells).

27. Method according to any of claims 7 to 26, further comprising isolating the population of clinically relevant lymphocytes from the expanded cell culture.

28. Method according to any of claims 7 to 27, wherein the testing for the presence of clinically relevant lymphocytes comprises using evaluation antigens.

29. Method according to claim 28, wherein evaluation antigens are presented to the cultured sample in a form selected from cells, in particular tumor cells, derived from the same mammal as the cultured sample (autologous cells), at least partially genetically matched allogeneic cells, in particular tumor cells, or cells expressing the clinically relevant antigens as a transgene.

30. Method according to claim 28 or 29, wherein the testing for the presence of clinically reactive lymphocytes comprises contacting the lymphocytes with at least one clinically relevant antigen and determining a change in either one of cytokine production, in particular INFγ production, cell proliferation, cytotoxicity, signaling and/or intracellular phosphorylation.

31. Immunotherapy for treating or preventing an infectious disease, a tumor disease, or an auto-immune disease in a mammal, comprising the steps of:
obtaining or generating a population of clinically relevant lymphocyte, according to the method of any of claims 7 to 30, wherein the body sample is obtained from said mammal; and
administering the population of clinically relevant lymphocytes to said mammal.

32. Immunotherapy according to claims 31, wherein the tumor disease is selected from a glioblastoma and pancreas cancer.

33. Immunotherapy according to claim 31 or 32, wherein the population of clinically relevant lymphocytes
bring about regression of cancer cells in the mammal;
interfere with the move from pre-malignant to malignant lesions;
bring about fast senescence of tumor cells or pre-malignant cells;
bring about removal of autoantigen-positive cells;
bring about killing, growth arrest or containment of pathogens;
interfere with cancer stem cells; and/or
induce growth arrest of cancer cells, or cells expressing auto-antigens.

34. Composition according to any of claims 1 to 6 for use in medical treatment, in particular for treating or preventing an infectious disease, an autoimmune disease or a tumor disease.

35. Composition for use according to claim 34, wherein the use comprises a generation of a population of clinically relevant lymphocytes with the method according to any of claims 7 to 31.

36. Kit for use in immunotherapy, in particular treatment of a tumor disease, wherein the kit comprises IL-2, IL-15, and IL-21 and optionally at least one of a component that stimulates the TCR, in particular OKT3, costimulatory molecules, feeder cells and a peptide comprising at the sequence of at least one clinically relevant antigen.

37. Clinically relevant lymphocyte obtained by a method according to any of claims 7 to 30, wherein the clinically relevant lymphocyte is selected from a B-cell, an NK cell and T-cell wherein the T-cell is selected from a helper T-cell (T_{H}-cell or CD4⁺−T-cell), in particular a T_{H1}-cell, a cytotoxic T-cell (T_{C}-cell or CD8⁺⁻T-cell), in particular CD8⁺CXCR3⁺ T-cell, a memory T-cell, in particular a central memory T-cell (T_{CM}-cell), stem memory T-cell (T_{SCM}) or peripheral memory cell (T_{PM}-cell), a gamma-delta T-cell (γδ-T-cell), a NK-T-cell, a Mucosal-associated invariant T-cell (MAiT), a double-negative T-cell (CD3⁺CD4⁻CD8⁻T-cell).

38. Clinically relevant lymphocyte according to claim 36 or 37, wherein the lymphocyte is selected from
a lymphocyte that expresses molecules that facilitate entry into tissues, in particular tumor- or infected or inflamed tissue (e.g. CXCR3); and
a lymphocyte that is enriched for markers of any of long-term memory, in particular CD117 and c-kit), activation of antigen-specific immune responses, in particular 4-1BB, cytolytic immune cell responses, in particular CD107a.

39. Lymphocyte obtained by a method according to any of claims 7 to 30,
expressing molecules and cytokines promoting the formation of a combination of lymphocytes useful for medical application, comprising T-cell precursors, T_{CM} and/or T_{PM};
expressing molecules and cytokines that promote the expansion of clinically relevant lymphocytes,
producing cytokines selected from IFN₆₅ , TNFα, IL-2, IL-17 and any combination thereof.
is a CD3+CD4−CD8−T-cell.

40. Population of clinically relevant lymphocytes obtained by a method according to any of claims 7 to 30.

41. Population of lymphocytes obtained by a method according to any of claims 7 to 30 comprising a population of clinically relevant lymphocytes.

42. Population of lymphocytes according to claim 41, characterized by one or more of the following features:
the percentage of T_{reg} based on the total number of T cells is below 5%, preferably below 3%;
the percentage of T_{H1}-cells based on the total number of T_{H}-cells is at least 50%, preferably at least 70%, more preferably at least 80%,
the percentage of CXCR3+ T-cells based on the total number of CD8+ T-cells is at least 50%, preferably at least 70%, more preferably at least 80%,
the percentage of 4-1 BB+ T-cells based on the total number of T-cells is at least 1%, preferably at least 2%, more preferably at least 2.5%,
the percentage of CD117+ T-cells based on the total number of T-cells is at least 1%, preferably at least 2%, more preferably at least 2.5%,
the percentage of CD3+CD4−CD8− cells based on the total number of T-cells is at least 1%, preferably at least 3%, more preferably at least 5%; and
the percentage of γδT-cells based on the total number of T-cells is at least 1%, preferably at least 3%, more preferably at least 5%.

43. Population of lymphocytes according to claim 41 or 42, characterized by one or more of the following features:
the percentage of precursor T-cells (CD45RA+CCR7+) based on the total number of T-cells is at least 1%, preferably at least 2%, more preferably at least 3%;
the percentage of central memory T-cells (CD45RA−CCR7+) based on the total number of T-cells is at least 2%, preferably at least 5%, more preferably at least 10%;
the percentage of peripheral memory T-cells (CD45RA−CCR7−) based on the total number of T-cells is at least 2%, preferably at least 5%, more preferably at least 10%; and
the percentage of effector T-cells (CD45RA+CCR7−) based on the total number of T-cells is at least 1%, preferably at least 3% more preferably at least 5%.

44. Population of lymphocytes according to any of claims 41 to 43, characterized by one or more of the following features:
the percentage of clinically relevant T-cells is at least 0.1% based on the total number of T-cells determined by multimeric soluble MHC-peptide complexes or intracellular cytokine production;
the intracellular cytokine production after antigen stimulation has a value that is at least 2-fold of the standard deviation without antigen stimulation; and
the CD107a induction after antigen stimulation has a value that is at least 2-fold of the standard deviation without antigen stimulation.


SEQUENCE LISTING (15 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 155 aa, Homo sapiens
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 5 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 6 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 7 - protein, 1210 aa, Homo sapiens [sequence omitted]

SEQ ID NO: 8 - protein, 510 aa, Homo sapiens
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKY

SEQ ID NO: 9 - protein, 944 aa, Homo sapiens
MDKDRPGPPALDDNMEEEVPSTSVVQEQVSAGDWENVLIELSDSSSEKEAEDAHLEPAQKGTKRKRVDHDAGGSAPARPMLPPQPDLPGREAILRRFPLDLRTLLQAIGAAATRIDTRAIDQFFGSQISNTEMYIMYAMAIRQAIRDRRRNPASRRDQAKWRLQTLAAGWPMGYQAYSSWMYSYTDHQTTPTFVHLQATLGCTGGRRCHVTFSAGTFKLPRCTPGDRQWLYVQSSVGNIVQSCNPRYSIFFDYMAIHRSLTKIWEEVLTPDQRVSFMEFLGFLQRTDLSYIKSFVSDALGTTSIQTPWIDDNPSTETAQAWNAGFLRGRAYGIDLLRTEGEHVEGATGETREESEDTESDGDDEDLPCIVSRGGPKVKRPPIFIRRLHRLLLMRAGKRTEQGKEVLEKARGSTYGTPRPPVPKPRPEVPQSDETATSHGSAQVPEPPTIHLAAQGMAYPLHEQHGMAPCPVAQAPPTPLPPVSPGDQLPGVFSDGRVACAPVPAPAGPIVRPWEPSLTQAAGQAFAPVRPQHMPVEPVPVPTVALERPVYPKPVRPAPPKIAMQGPGETSGIRRARERWRPAPWTPNPPRSPSQMSVRDRLARLRAEAQVKQASVEVQPPQLTQVSPQQPMEGPLVPEQQMFPGAPFSQVADVVRAPGVPAMQPQYFDLPLIQPISQGAPVAPLRASMGPVPPVPATQPQYFDIPLTEPINQGASAAHFLPQQPMEGPLVPEQWMFPGAALSQSVRPGVAQSQYFDLPLTQPINHGAPAAHFLHQPPMEGPWVPEQWMFQGAPPSQGTDVVQHQLDALGYTLHGLNHPGVPVSPAVNQYHLSQAAFGLPIDEDESGEGSDTSEPCEALDLSIHGRPCPQAPEWPVQEEGGQDATEVLDLSIHGRPRPRTPEWPVQGEGGQNVTGPETRRVVVSAVVHMCQDDEFPDLQDPPDEA

SEQ ID NO: 10 - protein, 641 aa, Homo sapiens
MSDEGPGTGPGNGLGEKGDTSGPEGSGGSGPQRRGGDNHGRGRGRGRGRGGGRPGAPGGSGSGPRHRDGVRRPQKRPSCIGCKGTHGGTGAGAGAGGAGAGGAGAGGGAGAGGGAGGAGGAGGAGAGGGAGAGGGAGGAGGAGAGGGAGAGGGAGGAGAGGGAGGAGGAGAGGGAGAGGGAGGAGAGGGAGGAGGAGAGGGAGAGGAGGAGGAGAGGAGAGGGAGGAGGAGAGGAGAGGAGAGGAGAGGAGGAGAGGAGGAGAGGAGGAGAGGGAGGAGAGGGAGGAGAGGAGGAGAGGAGGAGAGGAGGAGAGGGAGAGGAGAGGGGRGRGGSGGRGRGGSGGRGRGGSGGRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGTALAIPQCRLTPLSRLPFGMAPGPGPQPGPLRESIVCYFMVFLQTHIFAEVLKDAIKDLVMTKPAPTCNIRVTVCSFDDGVDLPPWFPPMVEGAAAEGDDGDDGDEGGDGDEGEEGQE

SEQ ID NO: 11 - protein, 505 aa, Homo sapiens
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 12 - protein, 1718 aa, Homo sapiens
MNQNTTEPVAATETLAEVPEHVLRGLPEEVRLFPSAVDKTRIGVWATKPILKGKKFGPFVGDKKKRSQVKNNVYMWEVYYPNLGWMCIDATDPEKGNWLRYVNWACSGEEQNLFPLEINRAIYYKTLKPIAPGEELLVWYNGEDNPEIAAAIEEERASARSKRSSPKSRKGKKKSQENKNKGNKIQDIQLKTSEPDFTSANMRDSAEGPKEDEEKPSASALEQPATLQEVASQEVPPELATPAPAWEPQPEPDERLEAAACEVNDLGEEEEEEEEEDEEEEEDDDDDELEDEGEEEASMPNENSVKEPEIRCDEKPEDLLEEPKTTSEETLEDCSEVTPAMQIPRTKEEANGDVFETFMFPCQHCERKFTTKQGLERHMHIHISTVNHAFKCKYCGKAFGTQINRRRHERRHEAGLKRKPSQTLQPSEDLADGKASGENVASKDDSSPPSLGPDCLIMNSEKASQDTINSSVVEENGEVKELHPCKYCKKVFGTHTNMRRHQRRVHERHLIPKGVRRKGGLEEPQPPAEQAQATQNVYVPSTEPEEEGEADDVYIMDISSNISENLNYYIDGKIQTNNNTSNCDVIEMESASADLYGINCLLTPVTVEITQNIKTTQVPVTEDLPKEPLGSTNSEAKKRRTASPPALPKIKAETDSDPMVPSCSLSLPLSISTTEAVSFHKEKSVYLSSKLKQLLQTQDKLTPAGISATEIAKLGPVCVSAPASMLPVTSSRFKRRTSSPPSSPQHSPALRDFGKPSDGKAAWTDAGLTSKKSKLESHSDSPAWSLSGRDERETVSPPCFDEYKMSKEWTASSAFSSVCNQQPLDLSSGVKQKAEGTGKTPVQWESVLDLSVHKKHCSDSEGKEFKESHSVQPTCSAVKKRKPTTCMLQKVLLNEYNGIDLPVENPADGTRSPSPCKSLEAQPDPDLGPGSGFPAPTVESTPDVCPSSPALQTPSLSSGQLPPLLIPTDPSSPPPCPPVLTVATPPPPLLPTVPLPAPSSSASPHPCPSPLSNATAQSPLPILSPTVSPSPSPIPPVEPLMSAASPGPPTLSSSSSSSSSSSSFSSSSSSSSPSPPPLSAISSVVSSGDNLEASLPMISFKQEELENEGLKPREEPQSAAEQDVVVQETFNKNFVCNVCESPFLSIKDLTKHLSIHAEEWPFKCEFCVQLFKDKTDLSEHRFLLHGVGNIFVCSVCKKEFAFLCNLQQHQRDLHPDKVCTHHEFESGTLRPQNFTDPSKAHVEHMQSLPEDPLETSKEEEELNDSSEELYTTIKIMASGIKTKDPDVRLGLNQHYPSFKPPPFQYHHRNPMGIGVTATNFTTHNIPQTFTTAIRCTKCGKGVDNMPELHKHILACASASDKKRYTPKKNPVPLKQTVQPKNGVVVLDNSGKNAFRRMGQPKRLNFSVELSKMSSNKLKLNALKKKNQLVQKAILQKNKSAKQKADLKNACESSSHICPYCNREFTYIGSLNKHAAFSCPKKPLSPPKKKVSHSSKKGGHSSPASSDKNSNSNHRRRTADAEIKMQSMQTPLGKTRARSSGPTQVPLPSSSFRSKQNVKFAASVKSKKPSSSSLRNSSPIRMAKITHVEGKKPKAVAKNHSAQLSSKTSRSLHVRVQKSKAVLQSKSTLASKKRTDRFNIKSRERSGGPVTRSLQLAAAADLSENKREDGSAKQELKDFSYSLRLASRCSPPAAPYITRQYRKVKAPAAAQFQGPFFKE

SEQ ID NO: 13 - protein, 230 aa, Homo sapiens
MEGQRWLPLEANPEVTNQFLKQLGLHPNWQFVDVYGMDPELLSMVPRPVCAVLLLFPITEKYEVFRTEEEEKIKSQGQDVTSSVYFMKQTISNACGTIGLIHAIANNKDKMHFESGSTLKKFLEESVSMSPEERARYLENYDAIRVTHETSAHEGQTEAPSIDEKVDLHFIALVHVDGHLYELDGRKPFPINHGETSDETLLEDAIEVCKKFMERDPDELRFNAIALSAA

SEQ ID NO: 14 - protein, 244 aa, Homo sapiens
MNGPADGEVDYKKKYRNLKRKLKFLIYEHECFQEELRKAQRKLLKVSRDKSFLLDRLLQYENVDEDSSDSDATASSDNSETEGTPKLSDTPAPKRKRSPPLGGAPSPSSLSLPPSTGFPLQASGVPSPYLSSLASSRYPPFPSDYLALQLPEPSPLRPKREKRPRLPRKLKMAVGPPDCPVGGPLTFPGRGSGAGVGTTLTPLPPPKMPPPTILSTVPRQMFSDAGSGDDALDGDDDLVIDIPE

SEQ ID NO: 15 - protein, 271 aa, Homo sapiens
MNLLGSRRVFSKKCRLVKFSMVALVSATMAVTTVTLENTALARQTQVSNDVVLNDGASKYLNEALAWTFNDSPNYYKTLGTSQITPALFPKAGDILYSKLDELGRTRTARGTLTYANVEGSYGVRQSFGKNQNPAGWTGNPNHVKYKIEWLNGLSYVGDFWNRSHLIADSLGGDALRVNAVTGTRTQNVGGRDQKGGMRYTEQRAQEWLEANRDGYLYYEVAPIYNADELIPRAVVVSMQSSDNTINEKVLVYNTANGYTINYHNGTPTQK